(19) United States
(12) Patent Application Publication
(10) Pub. No.: US 2023/0357430 A1
(43) Pub. Date: Nov. 9, 2023

(54) RNF167 AND CASTOR1 AS NOVEL MTOR TARGETS

(71) Applicant: UNIVERSITY OF PITTSBURGH - OF THE COMMONWEALTH SYSTEM OF HIGHER EDUCATION, Pittsburgh, PA (US)

(72) Inventor: Shou-Jiang Gao, Pittsburgh, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 18/335,235

(22) Filed: Jun. 15, 2023

Related U.S. Application Data
(63) Continuation of application No. PCT/US21/55296, filed on Oct. 15, 2021.
(60) Provisional application No. 63/126,922, filed on Dec. 17, 2020.

(51) Int. Cl. C07K 16/30; A61K 45/06; C07K 14/47

(52) U.S. Cl. CPC C07K 16/30; A61K 45/06; C07K 14/4703

(57) ABSTRACT

The present subject matter relates to the use of one or more inhibitors to treat a disease, e.g., cancer, in a subject. It is based, at least in part, on the discovery that protein kinase B (AKT) and ring finger protein 167 (RNF167)-mediated CASTOR1 degradation activates the mammalian target of rapamycin complex 1 (mTORC1) independent of arginine and promotes cancer progression. Accordingly, the presently disclosed subject matter provides for compositions, methods, and kits for treating a subject using an RNF167 inhibitor, an inhibitor that reduces phosphorylation of CASTOR1 at S14, ubiquitination and/or degradation of CASTOR1, or a combination thereof.

### CROSS-REFERENCE TO RELATED APPLICATION

This application is a continuation of International Patent Application No. PCT/US2021/055296, filed Oct. 15, 2021, which claims priority to U.S. Provisional Patent Application No. 63/126,922, filed on Dec. 17, 2020, the contents of each of which are incorporated in their entireties, and to each of which priority is claimed.

### STATEMENT OF GOVERNMENTAL INTEREST

This invention was made with government support under grant CA096512 awarded by the National Institutes of Health. The government has certain rights in the invention.

### SEQUENCE LISTING

The instant application contains a Sequence Listing, which has been electronically submitted in XML format and is hereby incorporated by reference in its entirety. Said XML copy, created on Jun. 17, 2023, is named 072396_0968_SL.xml and is 44,561 bytes in size.

### INTRODUCTION

The present disclosure relates to the use of inhibitors and/or agonists of proteins, e.g., RNF167 and CASTOR1, that regulate mTOR activity, and pharmaceutical compositions and kits thereof.

### BACKGROUND

The serine/threonine kinase AKT is mutated in about 10% of human cancer based on The Cancer Genome Atlas (TCGA) database, which largely accounts for its oncogenicity in these cancers. Other than mutations in the AKT gene, the dysregulation of upstream pathways of growth factors often activates AKT in cancer cells. For example, the dysregulation of estrogen receptor (ER), progesterone, and human epidermal growth factor 2 (HER2) lead to constitutive AKT phosphorylation and activation in more than 80% breast cancers. AKT has more than one hundred substrates. Among them, three major downstream nodes, including GSK3β, FOXOs and TSC2, mediate AKT's diverse functions in response to different stimulations. The most prominent consequence of AKT-mediated phosphorylation of a given protein is cellular translocation (e.g., FOXOs), degradation (e.g., GSK3β and TSC2), or alteration of protein-protein interaction (e.g., TSC2). AKT-mediated phosphorylation and inhibition of TSC2 have been described as the primary mechanism of AKT activation of the mammalian target of rapamycin complex 1 (mTORC1).

mTORC1, a central controller of cell proliferation in response to growth factors and nutrients, can be dysregulated in cancer. Whereas arginine activates mTORC1, it is overridden by high expression of cytosolic arginine sensor for mTORC1 subunit 1 (CASTOR1). Upon arginine deprivation, CASTOR1 interacts with and sequesters the positive regulator of mTORC1, the GATOR2 complex. In contrast, arginine stimulation releases GATOR2 from CASTOR1 and subsequently activates mTORC1. A high level of CASTOR1 protein inhibits mTORC1 activation by amino acids including arginine. Of note, tumor cells often have limited access to exogenous nutrients including amino acids, glucose, and oxygen. In particular, argininosuccinate synthase 1 (ASS1), the rate-limiting enzyme for endogenous arginine de novo synthesis, is silenced in up to 90% of cancer, rendering cancer cells arginine auxotrophic. Since cancer cells have constitutively activated mTORC1 and often encounter nutrient starvation, it is expected that the expression and function of CASTOR1 are inhibited by an alternative mechanism to arginine. It has been previously reported that viral microRNAs target CASTOR1 to activate mTORC1. As no specific CASTOR1 mutation associated with cancer has been described so far, how other cancer cells evade the inhibitory effect of CASTOR1 on mTORC1 in a nutrient deficient, especially amino acids deficient, tumor microenvironment in other types of cancer, remains unclear.

Therefore, there remains a need in the art for targeting novel pathways and proteins for improved cancer treatments.

### SUMMARY

The disclosed subject matter relates to methods for treating a disease of a subject, e.g., for treating a cancer or tumor of a subject. The present disclosure further provides pharmaceutical compositions and kits for use according to the disclosed methods.

In certain embodiments, the present disclosure provides methods for treating a disease in a subject in need thereof. In certain embodiments, the method includes administering a therapeutically effective amount of a ring finger protein 167 (RNF167) inhibitor to the subject. In certain embodiments, the RNF167 inhibitor can be selected from the group consisting of a compound, a small molecule, a chemical, a polypeptide, a peptide, a protein, and a combination thereof.

In certain embodiments, a method for treating a disease in a subject in need thereof includes administering a therapeutically effective amount of an inhibitor that reduces phosphorylation of CASTOR1, e.g., phosphorylation at S14, and/or reduces degradation of CASTOR1. In certain embodiments, a method for treating a disease in a subject in need thereof includes administering a therapeutically effective amount of a CASTOR1 agonist. In certain embodiments, the CASTOR1 agonist and/or inhibitor that reduces phosphorylation of CASTOR1, e.g., phosphorylation at S14, and/or reduces degradation of CASTOR1 can be selected from the group consisting of a compound, a small molecule, a chemical, a polypeptide, a peptide, a protein, and a combination thereof.

In certain embodiments, the disease can be diabetes. In certain non-limiting embodiments, the disease can be a cancer. In certain embodiments, the disease can be a breast cancer. In certain embodiments, the disease can be ageing.

In certain embodiments, methods of the present disclosure can further include administering a therapeutically effective amount of additional agent to the subject. In certain embodiments, the additional agent can be an anti-cancer agent. In certain embodiments, the additional agent can be a protein kinase B (AKT) inhibitor, a Tuberous Sclerosis Complex 2 (TSC2) inhibitor, an mTORC1 inhibitor or a combination thereof.

Another aspect of the present disclosure relates to methods for determining the prognosis of a subject having cancer. In certain embodiments, a method for determining the prognosis of a subject having cancer can include determining the expression level of RNF167 in a cancer of a subject and comparing the expression level of RNF167 in the cancer to a reference control level of RNF167, wherein increased expression of RNF167 in the cancer is indicative of a poor prognosis.

Alternatively or additionally, a method for determining the prognosis of a subject having cancer can include determining the expression level of CASTOR1 in a cancer of a subject and comparing the expression level of CASTOR1 in the cancer to a reference control level of CASTOR1, wherein decreased expression of CASTOR1 in the cancer is indicative of a poor prognosis. In certain embodiments, a method for determining the prognosis of a subject having cancer can include determining the level of CASTOR1 phosphorylation and/or ubiquitination in a cancer of a subject and comparing the level of CASTOR1 phosphorylation and/or ubiquitination in the cancer to a reference control level of CASTOR1, wherein an increased level of phosphorylation and/or ubiquitination of CASTOR1 in the cancer is indicative of a poor prognosis. In certain embodiments, the level of phosphorylation and/or ubiquitination of CASTOR1 is determined using an antibody or fragment thereof that binds to a phosphorylation and/or ubiquitination site of CASTOR1, e.g., the phosphorylation site can be amino acid S14 of CASTOR1 and/or the ubiquitination site can be selected from amino acids K61, K96 and/or K213 of CASTOR1. In certain embodiments, the subject has been previously treated with an anti-cancer treatment. In certain embodiments, the method further includes treating the subject with an anti-cancer treatment. In certain embodiments, the anti-cancer treatment comprises an mTOR inhibitor.

The presently disclosed subject matter further provides pharmaceutical compositions for treating a disease, e.g., a cancer, in a subject. In certain embodiments, a pharmaceutical composition for treating a disease in a subject can include a therapeutically effective amount of a ring finger protein 167 (RNF167) inhibitor. In certain embodiments, a pharmaceutical composition for treating a disease in a subject can include a therapeutically effective amount of an inhibitor that reduces the phosphorylation of CASTOR1, e.g., phosphorylation at S14, and/or reduces degradation of CASTOR1 and/or a CASTOR1 agonist. In certain embodiments, the RNF167 inhibitor, the CASTOR1 agonist and/or the inhibitor that reduces the phosphorylation of CASTOR1, e.g., phosphorylation at S14, and/or reduces degradation of CASTOR1 can be selected from the group consisting of a compound, a small molecule, a chemical, a polypeptide, a peptide, a protein, and a combination thereof.

The disclosed subject matter further provides kits for treating a disease in a subject. In certain embodiments, a kit for treating a disease in a subject can include a therapeutically effective amount of a ring finger protein 167 (RNF167) inhibitor, a therapeutically effective amount of a CASTOR1 agonist and/or a therapeutically effective amount of an inhibitor that reduces phosphorylation of CASTOR1, e.g., phosphorylation at S14, and/or reduces degradation of CASTOR1. In certain embodiments, the RNF167 inhibitor and/or the inhibitor that reduces phosphorylation of CASTOR1, e.g., phosphorylation at S14, the CASTOR1 agonist and/or reduces degradation of CASTOR1 can be selected from the group consisting of a compound, a small molecule, a chemical, a polypeptide, a peptide, a protein, and a combination thereof.

The disclosed subject matter provides kits for determining the prognosis of a subject having cancer. In certain embodiments, a kit for determining the prognosis of a subject having cancer can include means for detecting ring finger protein 167 (RNF167) and/or CASTOR1. In certain embodiments, the means for detecting RNF167 and/or CASTOR1 comprises one or more primers, probes, arrays/microarray, antibodies and/or bead for detecting RNF167 and/or CASTOR1. In certain embodiments, the means for detecting CASTOR1 comprises an antibody that specifically binds to phosphorylated or non-phosphorylated S14 of CASTOR1. In certain embodiments, a kit for determining the prognosis of a subject having cancer further includes (i) a therapeutically effective amount of a ring finger protein 167 (RNF167) inhibitor; (ii) a therapeutically effective amount of an inhibitor that reduces phosphorylation of CASTOR1 at S14 and/or reduces degradation and/or ubiquitination of CASTOR1; and/or (iii) a therapeutically effective amount of an agonist of CASTOR1.

In certain embodiments, the cancer can be selected from the group consisting of breast invasive carcinoma, brain lower grade glioma, skin cutaneous melanoma, head and neck squamous cell carcinoma, bladder carcinoma, fibroadenoma, gall bladder adenocarcinoma, testis seminoma, thyroid adenoma, fallopian tube adenocarcinoma, cervical squamous cell carcinoma and endocervical adenocarcinoma, lung adenocarcinoma, liver hepatocellular carcinoma, pancreatic adenocarcinoma, glioblastoma multiforme, and acute myeloid leukemia.

### BRIEF DESCRIPTION OF THE DRAWINGS

FIGS. 1A-1J provides protein analysis images showing that RNF167 mediates K29-linked polyubiquitination and degradation of CASTOR1 in response to growth factors. FIG. 1A shows the kinetics of CASTOR1 protein level and activation statuses of AKT and mTORC1 following fetal bovine serum (FBS) deprivation in 293T cells. FIG. 1B shows the kinetics of CASTOR1 protein level and activation statuses of AKT and mTORC1 following arginine deprivation in 293T cells. FIG. 1C provides a protein analysis image showing the CASTOR1 protein level, and activation statuses of AKT and mTORC1 following deprivation of FBS, arginine or both, or treatment with AKT inhibitor MK2206 in 293T cells. FIG. 1D provides a protein analysis image showing CASTOR1 ubiquitination status following deprivation of FBS, arginine or both. FIG. 1E provides a protein analysis image showing that CASTOR1 is labeled by K29-linked polyubiquitination, where a ubiquitin mutant K29 contained only the K29 lysine residue was sufficient to cause CASTOR1 polyubiquitination while mutation of K29 (K29R) abolished CASTOR1 polyubiquitination. FIGS. 1F and 1G provide protein analysis images showing that ectopic expression of RNF167 increases (FIG. 1F), whereas knockdown of RNF167 decreases (FIG. 1G) CASTOR1 ubiquitination. FIGS. 1H and 11 provide protein analysis images showing that RNF167 knockdown increases (FIG. 1H), whereas RNF167 overexpression decreases (FIG. 11) CASTOR1 protein level. FIG. 1J provides a schematic depiction of the K29-marked polyubiquitination and degradation of CASTOR1 protein by RNF167 in response to FBS.

FIGS. 2A-2K provide protein analysis images showing that AKT1 phosphorylation of CASTOR1 promotes RNF167-mediated ubiquitination and degradation of CASTOR1. FIG. 2A provides a protein analysis image showing that deprivation of arginine or leucine activated AKT and increased CASTOR1 phosphorylation at S14, whereas deprivation of FBS or total amino acid inactivates AKT and reduces CASTOR1 phosphorylation at S14. FIG. 2B provides a protein analysis image showing that CASTOR1 S14 phosphorylation is markedly reduced following alanine substitution of S14. FIG. 2C provides a protein analysis image showing AKT inhibition significantly decreased CASTOR1 phosphorylation at S14 in vivo. FIG. 2D provides a protein analysis image showing that AKT1 directly phosphorylates CASTOR1 in vitro. FIGS. 2E and 2F provide protein analysis images showing AKT overexpression increases (FIG. 2E), while AKT1 knockdown decreases (FIG. 2F) CASTOR1 degradation. FIGS. 2G and 2H provide protein analysis images showing that AKT1 overexpression increases (FIG. 2G), and AKT1 knockdown decreases (FIG. 2H) CASTOR1 ubiquitination. FIG. 2I provides a protein analysis image showing that CASTOR1 S14D had increases ubiquitination level compared to WT and S14A. FIGS. 2J and 2K provide protein analysis images showing that phosphorylation of CASTOR1 at S14 significantly increases its affinity to RNF167 (FIG. 2J) and quantifications of results from three independent experiments were presented (FIG. 2K), *P<0.05, one-way ANOVA followed by Tukey post-hoc test (k).

FIGS. 3A-3H provide protein analysis images showing that high CASTOR1 protein level overrides arginine activation of mTORC1 in physiological conditions. FIG. 3A provides a protein analysis image showing that the response of mTORC1 activation to CASTOR1 overexpression in a dose-dependent manner with and without the presence of arginine in 293T cells, where a high level of CASTOR1 overrides arginine-mediated mTORC1 activation. FIG. 3B provides a protein analysis image showing that CASTOR1 protein expression levels in multiple cell types including human lobar bronchial epithelial cells (HLBEC), human small airway epithelial cells (HSAEC), 293T, Hela, and breast cancer cell lines MCF7 and T47D. FIGS. 3C and 3D provide protein analysis images showing that Hela cells, which has almost no detectable CASTOR1 protein and a high level of constitutively activated mTORC1, was minimal responsive to arginine regulation of mTORC1, including arginine deprivation for 80 min (FIG. 3C) and re-stimulation for 10 min following arginine deprivation for 50 min (FIG. 3D). FIG. 3E provides a protein analysis image showing that MCF7 cells are more responsive than T47D cells to arginine-mediated mTORC1 activation, which was inversely correlated with their CASTOR1 protein levels as shown in FIG. 3B. FIG. 3F provides a protein analysis image showing that cells with high endogenous CASTOR1 protein levels including HSAEC and HLBEC are not responsive to arginine-mediated mTORC1 activation. FIG. 3G provides a protein analysis image showing that CASTOR1 knockdown in T47D cells, which have a high level of endogenous CASTOR1 protein activated mTORC1. FIG. 3H provides a summary of the relative endogenous CASTOR1 protein expression levels in different types of cells and their responsiveness to arginine regulation of mTORC1.

FIGS. 4A-4H provide protein analysis images showing that AKT-mediated phosphorylation and RNF167-mediated ubiquitination of CASTOR1 release mTORC1 inactivation by CASTOR1. FIG. 4A provides a protein analysis image showing that CASTOR1 binds to MIOS in a dose-dependent manner. FIG. 4B provides a protein analysis image showing that overexpression of RNF167 decreases CASTOR1 protein level and activated mTORC1 with and without the presence of arginine. FIG. 4C provides a protein analysis image showing that overexpression of a myristoylated constitutively active AKT1 (myr) but not the kinase-dead AKT1 mutant (K179M) reduces CASTOR1 protein level, decreases its binding to MIOS, and activates mTORC1. FIG. 4D provides a protein analysis image showing that AKT regulates mTORC1 activity by suppressing CASTOR1 was independent of TSC2. FIGS. 4E, 4F, and 4G provides protein analysis images showing that CASTOR1 S14D has a weaker binding to MIOS shown by CASTOR1 co-immunoprecipitation (co-IP) of MIOS (FIG. 4E) and reversed MIOS co-IP of CASTOR1 (FIG. 4G), hence S14D has a less inhibitory effect on mTORC1 than WT and S14A have (FIGS. 4E and 4F), and quantifications of results from two independent experiments shown in (FIG. 4F). FIG. 4H provides an illustration depicting that AKT phosphorylation and RNF167 ubiquitination of CASTOR1 reverse CASTOR1 inactivation of mTORC1.

FIGS. 5A-5J provide photographs and graphs showing that RNF167-mediated ubiquitination and AKT1-mediated phosphorylation of CASTOR1 promotes breast cancer progression. FIGS. 5A and 5B provide photographs and graphs showing that weaker suppression of colony formation of ER+ (FIG. 5A) and HER2+ (FIG. 5B) breast cancer cells in soft agar by CASTOR1 S14D than WT and S14A. FIG. 5C provides a photograph and a graph showing that CASTOR1 silencing enhanced colony formation in soft agar of T47D cells. FIGS. 5D-5G provide photographs and graphs showing that overexpression of CASTOR1 S14D have a less suppressive effect on breast tumor growth and a lower extended animal survival rate than WT and S14A had in a breast cancer xenograft model; the tumor volumes at the indicated time point post-inoculation are measured (FIG. 5D); the tumor volumes of the last time point are compared (FIG. 5E), and the actual tumors (FIG. 5F) and the survival rates (FIG. 5G) are shown. FIG. 5H-5J provides graphs showing that the CASTOR1 knockdown promoted tumor growth and shortened animal survival rate. The tumor volumes at the indicated time point post-inoculation are measured (FIG. 5H), and the tumor volumes of the last time point (FIG. 5I) and the survival rates (FIG. 5J) are shown.

FIGS. 6A-6L provide graphs and protein analysis images showing that CASTOR1 regulation by nutrients and growth factors in multiple cell lines. FIGS. 6A and 6B provide graphs showing that the CASTOR1 mRNA level slightly decreases by FBS (FIG. 6A) and arginine (FIG. 6B) deprivation in 293T cells. FIGS. 6C and 6D provide protein analysis images showing kinetics of CASTOR1 protein level and activation statuses of AKT and mTORC1 following FBS deprivation in MCF7 (FIG. 6C) and T47D (FIG. 6D) cells, where FBS deprivation, particularly for more than 16 h, increases CASTOR1 protein level. FIGS. 6E and 6F provide protein analysis images showing the kinetics of CASTOR1 protein level and activation statuses of AKT and mTORC1 following arginine deprivation in MCF7 (FIG. 6E) and T47D (FIG. 6F) cells. FIGS. 6G and 6H provide graphs showing the kinetics of CASTOR1 mRNA level following FBS deprivation in MCF7 (FIG. 6G) and T47D (FIG. 6H) cells. FIGS. 6I and 6J provide graphs showing the kinetics of CASTOR1 mRNA level following arginine deprivation in MCF7 (FIG. 6I) and T47D (FIG. 6J) cells. FIG. 6K provides a graph showing the CASTOR1 mRNA level following deprivation of FBS, arginine or leucine, or AKT inhibition. FBS, arginine, or leucine deprivation but not AKT inhibition decreased endogenous CASTOR1 mRNA level in 293T cells. FIG. 6L provides an image showing that arginine or leucine deprivation and S6K inhibitor inactivates mTORC, but only arginine or leucine deprivation rather than S6K inhibitor activates AKT and reduces CASTOR1 protein level.

FIGS. 7A-7H provide graphs and protein analysis images showing that RNF167 mediates K29-linked polyubiquitination and degradation of CASTOR1 in response to growth factors. FIG. 7A provides a protein analysis image showing that FBS starvation decreases while re-stimulation restored CASTOR1 ubiquitination. FIG. 7B provides a schematic illustration of the ubiquitin structure and the seven lysine residues in ubiquitin responsible for polyubiquitination linkage. FIG. 7C provides a protein analysis image showing that CASTOR1 is not tagged by K48- or K63-linked polyubiquitination. FIG. 7D provides an image showing the screening of E3 ligases that regulated the CASTOR1 protein level. FIG. 7E provides a protein analysis image showing that RNF167 overexpression increases CASTOR1 ubiquitination. FIGS. 7F and 7G provide graphs showing that RNF167 overexpression has no effect on the CASTOR1 mRNA level except at higher doses (>0.8 μg), which showed a marginal reduction (FIG. 7F), while RNF167 knockdown had no effect on CASTOR1 mRNA level (FIG. 7G). FIG. 7H provides an image showing that MG132 partially rescues RNF167-mediated CASTOR1 downregulation.

FIG. 8A-8M provide graphs and protein analysis images showing that CASTOR1 interacts with AKT and is phosphorylated by AKT at S14. FIGS. 8A-8D provide protein analysis images showing CASTOR1 interacted with exogenous (FIGS. 8A and 8C) and endogenous AKT (FIGS. 8B and 8D). FIG. 8E provides a schematic illustration depicting AKT1 domains consisting of PH and kinase domains, and a hydrophobic motif. FIG. 8F provides a protein analysis image showing that CASTOR1 interacts with the AKT1 kinase domain. FIG. 8G provides CASTOR1 protein sequences showing the AKT phosphorylation consensus motif in CASTOR1 is conserved among vertebrates. FIG. 8G discloses SEQ ID NOS 27-37, respectively, in order of appearance. FIG. 8H provides a protein analysis image showing that AKT interacts with and phosphorylated CASTOR1 in rat cells. FIG. 8I provides a protein analysis image showing Coomassie blue staining and immunoblotting analysis with an anti-GST antibody to examine the purity of recombinant GST-AKT1 and GST-CASTOR1 proteins. FIGS. 8J-8M provide graphs and protein analysis images showing that CASTOR1 S14D has stronger binding to AKT1 than WT and S14A had (FIG. 8J and FIG. 8L); results from two independent experiments were quantified and examined by one-way ANOVA followed by Tukey post-hoc test (FIGS. 8K and 8M).

FIGS. 9A-9H provide graphs and protein analysis images showing that AKT1-mediated phosphorylation of CASTOR1 promotes its proteasome-dependent degradation. FIG. 9A provides a protein analysis image showing that AKT1 kinase dead mutant K179M does not affect the CASTOR1 protein level. FIG. 9B provides a protein analysis image showing that the AKT1 kinase domain is sufficient to decrease the CASTOR1 protein level but to a lesser extent than the AKT1 WT. FIGS. 9C-9F provide graphs showing that myr-HA-AKT1, AKT1-K179M, different AKT1 domains, and different AKT1 siRNAs (siAKT1s) do not affect CASTOR1 mRNA level. FIGS. 9G and 9H provide protein analysis images showing that AKT1 accelerates CASTOR1 degradation. 293T cells co-transfected with Flag-CASTOR1 and myr-HA-AKT1 for 36 h are treated with either cycloheximide (CHX) (FIG. 9G) or MG132 (FIG. 9H).

FIGS. 10A-10E provide graphs and protein analysis images showing that AKT1-mediated phosphorylation of CASTOR1 promotes its proteasome-dependent degradation. FIG. 10A provides a protein analysis image showing that WT, S14A, and S14D have similar mRNA levels while the protein level of the S14D level was lower than those of WT and S14A. FIGS. 10B and 10C provide a protein analysis image and a graph showing that CASTOR1 S14D has a faster turnover than WT and S14A had. The protein level of CASTOR1 WT, S14A, or S14D are examined following treatment with either CHX or MG132 for the indicated time (FIG. 10B), and the relative levels are quantified and presented in (FIG. 10C). FIG. 10D provides a protein analysis image showing that S14D has a lower protein level following treatment with cycloheximide (CHX) but a higher protein level following treatment with MG132 than WT and S14A have. FIG. 10E provides a protein analysis image showing that CASTOR1 S14D has an increased ubiquitination level compared to WT and S14A.

FIGS. 11A-11E provide graphs and protein analysis images showing that CASTOR1 is marked by K29-linked polyubiquitination at K61, K96, and K213. FIG. 11A provides a schematic illustration of CASTOR1 structure and the three putative ubiquitination lysine residues responsible for polyubiquitination. FIGS. 11B and 11C provide images showing that simultaneous mutations of CASTOR1 lysines K61, K96 and K213 to arginine (3KR) were required to stabilize the protein (FIG. 11B) but had no effect on mRNA level (FIG. 11C). FIGS. 11D and 11E provide images showing that the K61, K96, and K213 triple mutant 3KR of Flag-CASTOR1 S14D was resistant to RNF167-mediated ubiquitination (FIG. 11D) and degradation (FIG. 11E).

FIGS. 12A-12I provide graphs showing that expression levels of CASTOR1 and RNF167 regulate mTORC1 and predict cancer survival in different types of cancer. FIGS. 12A and B provide graphs showing that a lower CASTOR1 expression level is associated with poor overall survival (FIG. 12A) and disease-free survival (FIG. 12B) in the pan-cancer analysis. FIG. 12C provides a graph showing that a lower CASTOR1 mRNA expression level is associated with poor survival in 10 types of cancer including breast invasive carcinoma (BRCA), brain lower grade glioma (LGG), skin cutaneous melanoma (SKCM), head and neck squamous cell carcinoma (HNSC), cervical squamous cell carcinoma and endocervical adenocarcinoma (CESC), lung adenocarcinoma (LUAD), liver hepatocellular carcinoma (LIHC), pancreatic adenocarcinoma (PAAD), glioblastoma multiforme (GBM) and acute myeloid leukemia (LAML). Analyses were performed with the TCGA database. FIG. 12D provides a graph showing that a high RNF167 mRNA expression level is associated with poor survival in types of cancer including GBM, LAML, SKCM, LGG, and LIHC. FIG. 12E provides a graph showing that the RNF167 expression level is higher in breast cancer tumors than the adjacent normal tissues. FIGS. 12F and 12G provide graphs showing that a lower CASTOR1 expression level is associated with poor survival of specific breast cancer subtypes including HER2-positive (HER2+) (FIG. 12F) and ER-positive (ER+) (FIG. 12G) subtypes. FIGS. 12H-12I provide graphs showing that a high RNF167 expression level is associated with poor survival of ER+(FIG. 12H) and HER2+ (FIG. 121) subtypes of breast cancer.

FIGS. 13A-13F provide protein analysis images showing that AKT1-mediated phosphorylation and degradation of CASTOR1 in breast cancer cells. FIG. 13A provides a protein analysis image showing that a CASTOR1 protein level is negatively correlated with AKT activation in ER+ and HER2+ breast cells, respectively. FIG. 13B provides a protein analysis image showing that CASTOR1 interacts with AKT1 in MCF7 cells. FIG. 13C provides a protein analysis image showing that AKT1 silencing increases the CASTOR1 protein level in ER+ breast cancer cells. FIG. 13D provides a protein analysis image showing that AKT inhibitor MK2206 increases the endogenous CASTOR1 protein level in ER+ breast cancer cells. FIGS. 13E-13F provide protein analysis images showing that overexpression of myr-HA-AKT1 (FIG. 13E) but not the AKT kinase dead mutant K179M (FIG. 13F) in MCF7 and T47D cells resulted in a dose-dependent reduction in CASTOR1 protein level.

FIGS. 14A-14E provide graphs and protein analysis images showing that AKT phosphorylation of CASTOR1 promotes RNF167-mediated CASTOR1 degradation in breast cancer cells. FIGS. 14A-14C provide graphs and protein analysis images showing that CASTOR1 S14D had a higher affinity to RNF167 than WT and S14A had in MCF7 cells (FIGS. 14A and 14B) and T47D cells (FIG. 14C). FIGS. 14D and 14E provide images showing that RNF167 overexpression (FIG. 14D) decreases while RNF167 knockdown (FIG. 14E) increases CASTOR1 expression in MCF7 cells.

FIGS. 15A-15J provide graphs and protein analysis images showing that CASTOR1 inhibits cell proliferation, cell cycle progression, and colony formation in soft agar of breast cancer cells by inactivating mTORC1. FIGS. 15A-15C provide protein analysis images showing that inhibition of mTORC1 activation is weaker by Flag-CASTOR1 S14D than WT and S14A in MCF7 (FIG. 15A), HCC1569 cells (FIG. 15B) and T47D (FIG. 15C). FIGS. 15D-15F provide graphs and images showing that CASTOR1 S14D has a weaker effect than WT and S14A has on suppressing cell proliferation in MCF cells (FIG. 15D) and HCC1569 cells (FIG. 15E), and colony formation in soft agar of T47D cells (FIG. 15F). FIGS. 15G-15H provide graphs showing that overexpression of Flag-CASTOR1 S14D induces weaker cell cycle arrest than WT, and S14A does in ER+ MCF7 (FIG. 15G) and HER2+ HCC1569 (FIG. 15H). FIGS. 15I-15J provide graphs showing that overexpression of Flag-CASTOR1 WT, S14A, or S14D has minimal effects on apoptosis in ER+ MCF7 cells (FIG. 151) and HER2+ HCC1569 cells (FIG. 15J).

FIGS. 16A-16B provide graphs showing that AKT1-mediated phosphorylation and degradation, as well as silencing of CASTOR1, promote breast cancer progression. FIG. 16A provides graphs showing that individual tumor growth curves showing that CASTOR1 WT and S14A have more inhibitory effects on tumor growth than S14D had. FIG. 16B provides graphs showing that the individual tumor growth curve after CASTOR1 silencing reveals that CASTOR1 deletion promotes tumor growth.

FIG. 17 provides a proposed model of AKT-mediated phosphorylation and RNF167-dependent ubiquitination of CASTOR1 and mTORC1 activation in normal and cancer cells.

FIGS. 18A-18D provide graphs and images showing that CASTOR1 is tumor suppressive in a genetic Kras-driven lung cancer model. FIG. 18A provides tissue images showing that CASTOR1 knockout (KO) mice develop larger tumors than WT CASTOR1 mice. No tumors are observed in WT CASTOR1 and CASTOR1 KO mice without Kras expression. FIG. 18B provides tissue images showing that CASTOR1 KO mice develop more foci and larger tumors than WT CASTOR1 mice. FIG. 18C provides a graph showing quantification of tumor foci and size. FIG. 18D provides a graph showing average tumor sizes of Kras-driven tumors in WT CASTOR1 and CASTOR1 KO mice.

FIGS. 19A-19C provide images showing that CASTOR1 KO tumors have more tumor cells and higher levels of mTORC1 activation and proliferation cells. FIG. 19A provides tissue images showing that tumors from CASTOR1 KO mice have more TTF1-positive tumor cells than those from CASTOR1 WT mice. FIG. 19B provides tissue images showing that tumors from CASTOR1 KO mice have higher levels of mTORC1 activation than those from CASTOR1 WT mice shown by p4EBP1 staining. FIG. 19C provides tissue images showing that tumors from CASTOR1 KO mice have more proliferation cells than those from CASTOR1 WT mice shown by Ki67 staining.

FIG. 20 provides images showing p-CASTOR1 in MCF7 cells of WT and S14D CASTOR1. Higher levels of p-CASTOR1 are detected in cells of WT and S14D CASTOR1 than cells with the vector control.

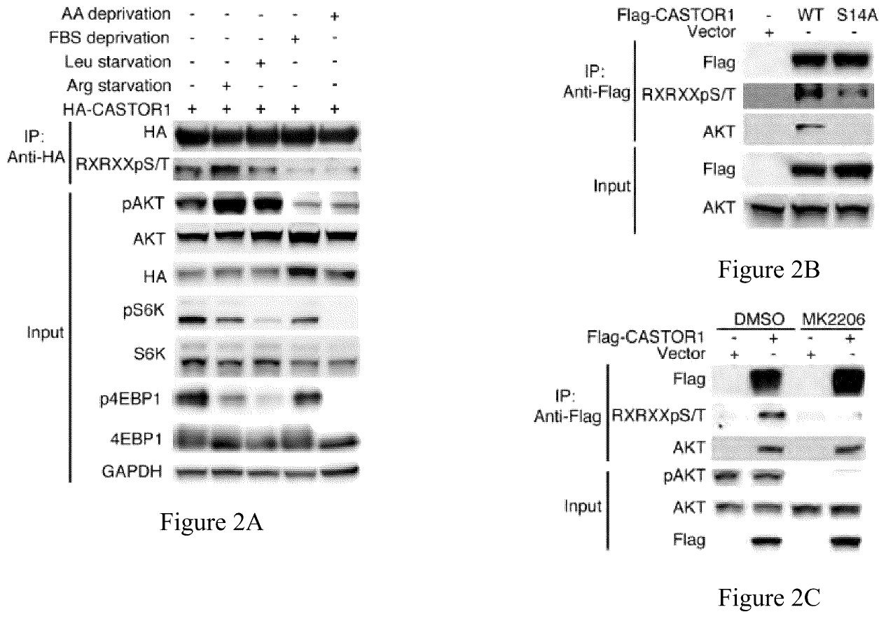

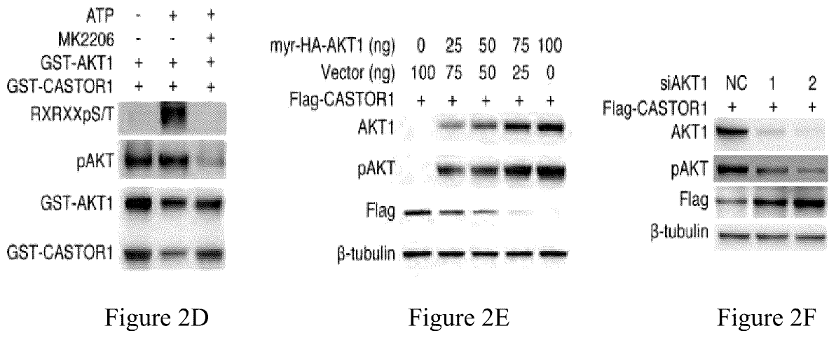

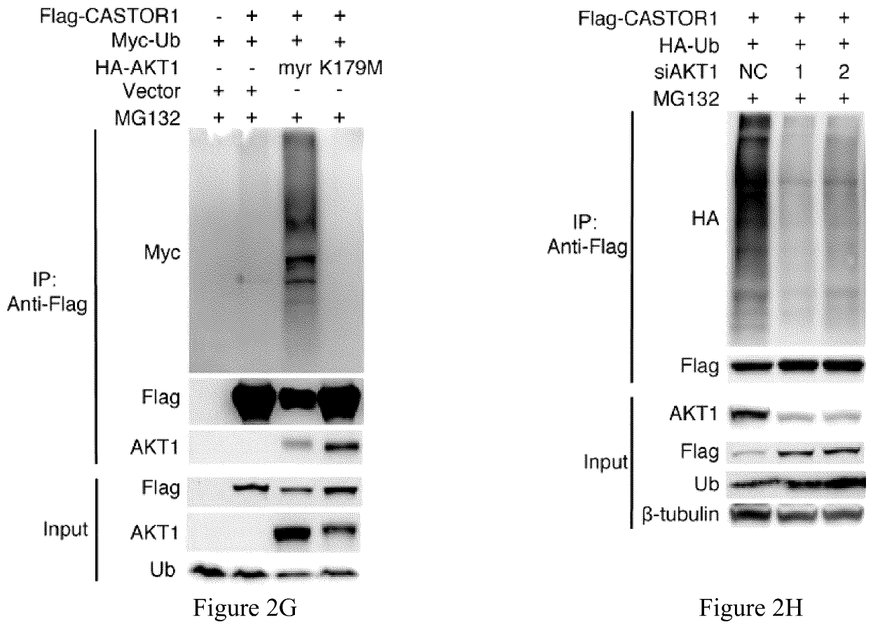

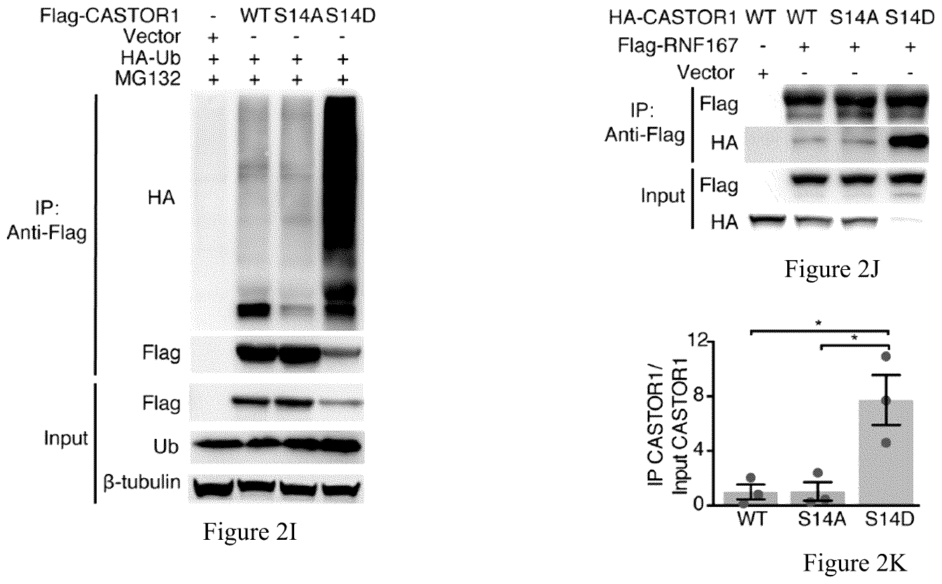

FIG. 2I provides images showing p-CASTOR1 expression in xenograft tumors of MCF7 cells of WT CASTOR1 and Vector control. Higher levels of p-CASTOR1 are detected in WT CASTOR1 tumors than in Vector control tumors.

FIG. 22 provides images showing p-CASTOR1 expression in tumors of Kras-driven mouse lung cancer model. p-CASTOR1 is detected in Kras-driven lung tumors of WT mice but not in Kras-driven lung tumors of CASTOR1 knockout mice. Lung tissues from WT mice and CASTOR1 knockout mice are free of tumors and are negative for p-CASTOR1.

FIG. 23 provides images showing p-CASTOR1 expression in lung adenocarcinoma and normal lung tissue. Higher levels of p-CASTOR1 are detected in tumors cells than in normal control cells.

FIG. 24 provides images showing p-CASTOR1 expression in bladder carcinoma and normal bladder tissue. Higher levels of p-CASTOR1 are detected in tumors cells than in normal control cells.

FIG. 25 provides images showing p-CASTOR1 expression in breast fibroadenoma and normal breast tissue. Higher levels of p-CASTOR1 are detected in tumors cells than in normal control cells.

FIG. 26 provides images showing p-CASTOR1 expression in fallopian tube adenocarcinoma and normal tissue. Higher levels of p-CASTOR1 are detected in tumors cells than in normal control cells.

FIG. 27 provides images showing p-CASTOR1 expression in gall bladder adenocarcinoma and normal tissue. Higher levels of p-CASTOR1 are detected in tumors cells than in normal control cells.

FIG. 28 provides images showing p-CASTOR1 expression in kidney clear cell carcinoma and normal tissue. Higher levels of p-CASTOR1 are detected in tumors cells than in normal control cells.

FIG. 29 provides images showing p-CASTOR1 expression in pancreas adenocarcinoma and normal tissue. Higher levels of p-CASTOR1 are detected in tumors cells than in normal control cells.

FIG. 30 provides images showing p-CASTOR1 expression in skin malignant melanoma and normal tissue. Higher levels of p-CASTOR1 are detected in tumors cells than in normal control cells.

FIG. 31 provides images showing p-CASTOR1 expression in testis seminoma and normal tissue. Higher levels of p-CASTOR1 are detected in tumors cells than in normal control cells.

FIG. 32 provides images showing p-CASTOR1 expression in thyroid adenoma and normal tissue. Higher levels of p-CASTOR1 are detected in tumors cells than in normal control cells.

FIG. 33 provides images showing CASTOR1 phosphorylation at S14 significantly inhibits CASTOR1 dimerization.

### DETAILED DESCRIPTION

The detailed description of the disclosed subject matter is divided into the following subsections for clarity and not by way of limitation:
I. Definitions;
II. Inhibitors and Agonists;
III. Methods of Use;
IV. Pharmaceutical Compositions; and
V. Kits.

### I. Definitions

The terms used in this specification generally have their ordinary meanings in the art, within the context of the disclosed subject matter and in the specific context where each term is used. Certain terms are discussed below, or elsewhere in the specification, to provide additional guidance to the practitioner in describing the compositions and methods of the disclosed subject matter.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “inhibitor” refers to a compound or molecule (e.g., small molecule, peptide, peptidomimetic, natural compound, siRNA, anti-sense nucleic acid, aptamer, or antibody) that interferes with (e.g., reduces, prevents, decreases, suppresses, eliminates or blocks) the signaling function of a protein or pathway. An inhibitor can be any compound or molecule that changes any activity of a protein (signaling molecule, any molecule involved with the named signaling molecule or a named associated molecule), such as RNF167, or interferes with the interaction of a protein, e.g., RNF167, with signaling partners. Inhibitors also include molecules that indirectly regulate the biological activity of a named protein, e.g., RNF167, by intercepting upstream signaling molecules.

The term “agent,” as used herein, means a substance that produces or is capable of producing an effect and would include, but is not limited to, chemicals, pharmaceuticals, biologics, small organic molecules, antibodies, nucleic acids, peptides and proteins.

The terms “inhibiting,” “eliminating,” “decreasing,” “reducing” or “preventing,” or any variation of these terms, referred to herein, includes any measurable decrease or complete inhibition to achieve a desired result.

An “effective amount” or “therapeutically effective amount” of an agent, e.g., an RNF167 inhibitor, refers to an amount effective, at dosages and for periods of time necessary, to achieve the desired therapeutic or prophylactic result, e.g., treating a cancer in a subject. A therapeutically effective amount can be administered in one or more administrations.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The term “in need thereof” would be a subject known or suspected of having or being at risk of developing a disease, e.g., cancer.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, prolonging survival, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., cancer/tumor.

An “anti-cancer/tumor effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse, or progression-free survival in a patient diagnosed with cancer.

An “anti-cancer/tumor agent,” as used herein, can be any molecule, compound, chemical, or composition that has an anti-cancer effect. Anti-cancer agents include, but are not limited to, chemotherapeutic agents, radiotherapeutic agents, cytokines, anti-angiogenic agents, apoptosis-inducing agents, anti-cancer antibodies and/or agents which promote the activity of the immune system. In certain embodiments, an anti-cancer agent can be a radiotherapeutic agent. In certain embodiments, an anti-cancer agent can be a chemotherapeutic agent. Other non-limiting exemplary anti-cancer agents that can be used with the presently disclosed subject matter include tumor-antigen based vaccines and chimeric antigen receptor T-cells.

As used herein, the term “disease” refers to any condition or disorder that damages or interferes with the normal function of a cell, tissue, or organ.

The terms “detection” or “detecting” include any means of detecting, including direct and indirect detection.

“Antibody,” “fragment of an antibody,” or “antibody fragment” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

As described herein, any concentration range, percentage range, ratio range or integer range is to be understood to include the value of any integer within the recited range and, when appropriate, fractions thereof (such as one-tenth and one-hundredth of an integer), unless otherwise indicated.

### II. Inhibitors and Agonists

The present disclosure relates to the use of inhibitors and agonists, e.g., inhibitors and agonists of proteins that indirectly and/or directly regulate mTORC1 activity, for preventing and/or treating a disease. In certain embodiments, the disease is a cancer and/or tumor, in a subject.

RING Finger Protein 167 (RNF167)

The present disclosure relates to the use of an inhibitor of RNF167 to treat a disease, e.g., a cancer and/or tumor, in a subject.

RNF167 is a RING-type E3 ligase involved in regulating protein trafficking, localization, and degradation by directly ubiquitinating targeted substrates. RNF167 is encoded by the RNF167 gene. In certain embodiments, RNF167 is a human RNF167 protein. In certain embodiments, RNF167 is a mouse RNF167 protein or a rat RNF167 protein.

In certain embodiments, RNF167 can be a human RNF167 protein having an amino acid sequence as set forth in GenBank Accession No. NP_056343 or an amino acid sequence at least about 95 percent or at least about 98 percent homologous thereto.

In certain embodiments, RNF167 can be an RNF167 protein that is post-translationally modified. For example, but not by way of limitation, the RNF167 protein can be glycosylated, e.g., at the N-terminus.

In certain embodiments, a nucleic acid encoding a human RNF167 protein of the present invention can comprise a nucleic acid sequence as set forth in GenBank Accession No. NM_015528.3 or a nucleic acid sequence at least about 95 percent or at least about 98 percent homologous thereto.

Any suitable RNF167 inhibitors known in the art can be used with the presently disclosed subject matter. Non-limiting exemplary RNF16 inhibitors can include any compounds, molecules, chemicals, polypeptides, proteins, peptides, agonists, and combinations thereof that inhibit and/or reduce the expression, function and/or activity of RNF167. In certain embodiments, the inhibitor can partially and/or completely inhibit the RNF167 signaling pathway and/or activity of RNF167. For example, but not by way of limitation, “partially inhibit” can refer to a reduction of at least 20%, at least 30%, at least 40%, at least 50%, at least 60%, at least 70%, at least 80%, at least 90%, at least 95%, at least 97% or at least 99% in the activity, function and/or expression of RNF167, e.g., as compared to a reference. In certain embodiments, the reference is a sample not treated with the RNF167 inhibitor.

In certain embodiments, the RNF167 inhibitor for use with the presently disclosed subject matter can be a small molecule compound that inhibits and/or reduces the expression, function and/or activity of RNF167 or a pharmaceutically acceptable salt or solvate thereof.

In certain embodiments, the RNF167 inhibitor can be a ribozyme, an antisense oligonucleotide, a shRNA molecule, and/or an siRNA molecule that specifically inhibits and/or reduces the expression, function and/or activity of RNF167. Non-limiting examples of such inhibitors are provided in the Example.

In certain embodiments, the RNF167 inhibitor can be an antisense nucleic acid, a shRNA, or a siRNA that is homologous to at least a portion of a RNF167 nucleic acid sequence. The homology of the portion relative to the RNF167 nucleic acid sequence can be at least about 50, at least about 55, at least about 60, at least about 65, at least about 70, at least about 75, at least about 80, at least about 85, at least about 90, at least about 95, or at least about 98 percent. The percent homology can be determined by, for example, BLAST or FASTA software. In certain embodiments, the homologous portion constitutes at least 10 nucleotides, at least 15 nucleotides, at least 20 nucleotides, at least 25 nucleotides, at least 30 nucleotides. In certain embodiments, the antisense nucleic acid, shRNA, or siRNA molecules have up to 15, up to 20, up to 25, up to 30, up to 35, up to 40, up to 45, up to 50, up to 75, or up to 100 nucleotides in length. In certain embodiments, the antisense nucleic acid, shRNA, or siRNA molecules comprise DNA or atypical or non-naturally occurring residues, for example, but not limited to, phosphorothioate residues.

In certain embodiments, the antisense nucleic acid, shRNA, or siRNA molecules disclosed herein can be expressed from a vector or produced chemically or synthetically. Methods for selecting an appropriate dsRNA or dsRNA-encoding vector are well known in the art for genes whose sequence is known (e.g., see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and PCT Patent Application Nos. WO 2001/036646, WO 1999/032619 and WO 2001/068836, the contents of which are incorporated by reference herein in their entireties).

In certain embodiments, the RNF167 inhibitor for use with the presently disclosed subject matter can be a peptide and/or a small protein fragment. For example, but not by way of limitation, a peptide and/or a small protein fragment that inhibits and/or reduces the expression, function and/or activity of RNF167 and/or partially or completely blocks the RNF167 signaling pathway or activity can be used as the RNF167 inhibitor.

In certain embodiments, the RNF167 inhibitor can be an antibody or an antibody fragment that partially or completely blocks RNF167 signaling and/or activity. For example, but not by way of limitation, an antibody (or fragment thereof) for use in the present disclosure can physically bind to RNF167 and/or bind to a protein that regulates the expression, activity and/or function of RNF167.

In certain embodiments, an RNF167 inhibitor of the present disclosure can be conjugated to a modality that specifically targets cancer cells. For example, and not by way of limitation, a RNF167 inhibitor can be conjugated to an antibody or antibody fragment and/or peptide, e.g., that recognizes an epitope on the surface of a cancer cell. In certain embodiments, the modality can be a nanoparticle that specifically targets cancer cells, e.g., by the presence of a targeting moiety conjugated to the nanoparticle.

Cytosolic Arginine Sensor for mTORC1 Subunit 1 (CASTOR1)

The present disclosure further relates to the use of an inhibitor that inhibits and/or reduces the phosphorylation, ubiquitination and/or degradation of CASTOR1 to treat a disease. The present disclosure further provides agonists of CASTOR1 that increase and/or enhance the activity, function and/or expression of CASTOR1. In certain embodiments, the disease is a cancer and/or tumor in a subject.

CASTOR1 functions as an arginine sensor for the mTORC1 pathway and contains a consensus AKT1 phosphorylation motif (e.g., R—V—R—V-L-S14 (SEQ ID NO: 26)). CASTOR1 is encoded by the CASTOR1 gene. In certain embodiments, CASTOR1 is a human CASTOR1 protein. In certain embodiments, CASTOR1 is a mouse CASTOR1 protein or a rat CASTOR1 protein.

In certain embodiments, CASTOR1 can be a human CASTOR1 protein having an amino acid sequence as set forth in GenBank Accession No. NP_001032755 or an amino acid sequence at least about 95 percent or at least about 98 percent homologous thereto.

In certain embodiments, a nucleic acid encoding a human CASTOR1 protein of the present invention can comprise a nucleic acid sequence as set forth in GenBank Accession No. NM_001037666.3 or a nucleic acid sequence at least about 95 percent or at least about 98 percent homologous thereto.

In certain embodiments, an inhibitor for use in the present disclosure can reduce or inhibit phosphorylation and/or ubiquitination of CASTOR1. In certain embodiments, an inhibitor for use in the present disclosure can reduce or inhibit phosphorylation of CASTOR1 at S14. In certain embodiments, an inhibitor for use in the present disclosure can reduce or inhibit the ubiquitination of CASTOR1. In certain embodiments, an inhibitor for use in the present disclosure can reduce or inhibit degradation of CASTOR1. In certain embodiments, an inhibitor for use in the present disclosure can reduce and/or inhibit the binding of an E3 ligase, e.g., RNF167, to CASTOR1.

In certain embodiments, an agonist of CASTOR1 can be an inhibitor as disclosed herein, e.g., an inhibitor that inhibits and/or reduces the phosphorylation, ubiquitination and/or degradation of CASTOR1 and/or an inhibitor that inhibits and/or reduces the expression, function and/or activity of RNF167 and/or the RNF167 signaling pathway.

In certain embodiments, the inhibitor can partially and/or completely inhibit the phosphorylation, degradation and/or ubiquitination of CASTOR1. For example, but not by way of limitation, “partially inhibit” can refer to a reduction of at least 20%, at least 30%, at least 40%, at least 50%, at least 60%, at least 70%, at least 80%, at least 90%, at least 95%, at least 97% or at least 99% in the phosphorylation, degradation and/or ubiquitination of CASTOR1, e.g., as compared to a reference. In certain embodiments, the reference is a sample not treated with the inhibitor.

In certain embodiments, an inhibitor that reduces or inhibits phosphorylation and/or ubiquitination of CASTOR1 and/or reduces and/or inhibits the binding of an E3 ligase to CASTOR1 results in an increase in the expression, activity and/or function of CASTOR1. For example, but not by way of limitation, an inhibitor can result in greater than a 1.5-fold increase, greater than about a 2-fold increase, greater than about a 2.5-fold increase, greater than about 3-fold increase or greater than about a 3.5-fold increase in CASTOR1 expression, activity and/or function, e.g., relative to the reference control level. In certain embodiments, the reference control level is the level of expression, activity and/or function of CASTOR1 in a sample not treated with the inhibitor.

Any suitable inhibitors for reducing and/or phosphorylation, ubiquitination and/or degradation of CASTOR1 known in the art can be used with the presently disclosed subject matter. Non-limiting exemplary inhibitors, which reduce and/or inhibit phosphorylation of CASTOR1, can include any compounds, molecules, chemicals, polypeptides, peptides, proteins, agonists, and combinations thereof.

In certain embodiments, the inhibitors for use with the presently disclosed subject matter are small molecule compounds that reduce or inhibit the phosphorylation, ubiquitination and/or degradation of CASTOR1 or a pharmaceutically acceptable salt or solvate thereof.

In certain non-limiting embodiments, a peptide and/or a small protein fragment that reduces and/or inhibits, e.g., partially or completely blocks, phosphorylation, ubiquitination and/or degradation of CASTOR1 can be used as an inhibitor. For example, but not by way of limitation, peptides and or protein fragments that can regulate the activity of CASTOR1 by modifying ubiquitination level and/or the binding of an E3 ligase (e.g., RNF167) can be used as an inhibitor.

In certain embodiments, the inhibitors, which reduce or inhibit phosphorylation, ubiquitination and/or degradation of CASTOR1, can be selected from the group consisting of ribozymes, antisense oligonucleotides, shRNA molecules, and siRNA molecules. In non-limiting embodiments, the inhibitor can be an antisense nucleic acid, shRNA, or siRNA that is homologous to at least a portion of a CASTOR1 nucleic acid sequence. The homology of the portion relative to the CASTOR1 sequence can be at least about 50, at least about 55, at least about 60, at least about 65, at least about 70, at least about 75, at least about 80, at least about 85, at least about 90, at least about 95, or at least about 98 percent. The percent homology can be determined by, for example, BLAST or FASTA software. In certain embodiments, the homologous portion constitutes at least 10 nucleotides, at least 15 nucleotides, at least 20 nucleotides, at least nucleotides, at least 30 nucleotides. In certain embodiments, the antisense nucleic acid, shRNA, or siRNA molecules have up to 15, up to 20, up to 25, up to 30, up to 35, up to 40, up to 45, up to 50, up to 75, or up to 100 nucleotides in length. In certain embodiments, the antisense nucleic acid, shRNA, or siRNA molecules comprise DNA or atypical or non-naturally occurring residues, for example, but not limited to, phosphorothioate residues.

In certain embodiments, the antisense nucleic acid, shRNA, or siRNA molecules disclosed herein can be expressed from a vector or produced chemically or synthetically. Methods for selecting an appropriate dsRNA or dsRNA-encoding vector are well known in the art for genes whose sequence is known.

In certain embodiments, the inhibitors, which reduce or inhibit phosphorylation of CASTOR1, e.g., at S14, ubiquitination and/or degradation of CASTOR1 can be an antibody or an antibody fragment. For example, but not by way of limitation, an antibody (or an antibody fragment) that reduces and/inhibits the phosphorylation of CASTOR1 can partially and/or completely block the interaction between CASTOR1 and a protein that induces the phosphorylation of CASTOR1, e.g., by binding CASTOR1 and/or binding the protein that induces the phosphorylation, ubiquitination and/or degradation of CASTOR1. In certain embodiments, an antibody (or an antibody fragment) that reduces and/inhibits CASTOR1 activity can partially and/or completely block the interaction between phosphorylated CASTOR1 and RNF167. For example, but not by way of limitation, the inhibitor can be an antibody or fragment thereof that selectively binds to a phosphorylation and/or ubiquitination site. For example, but not by way of limitation, the inhibitor can be an antibody or fragment thereof that specifically binds to the S14 phosphorylation site of CASTOR1. In certain embodiments, the inhibitor can be an antibody or fragment thereof that specifically binds to one or more CASTOR1 ubiquitination sites, e.g., K61, K96 and/or K213.

In certain embodiments, the inhibitors, which reduce and/or inhibit phosphorylation, ubiquitination and/or degradation of CASTOR1, of the present disclosure can be conjugated to a modality that specifically targets cancer cells. For example, and not by way of limitation, the inhibitor can be conjugated to an antibody or antibody fragment and/or peptide, e.g., that recognizes an epitope on the surface of a cancer cell. In certain embodiments, the modality can be a nanoparticle that specifically targets cancer cells, e.g., by the presence of a targeting moiety conjugated to the nanoparticle.

### II. Methods of Use

Prognostic Methods

The present disclosure provides methods for determining the prognosis of a patient that has cancer and/or a tumor. As described in detail in the Example section below, the studies presented in the instant application indicate that high RNF167 expression and/or low CASTOR1 expression are associated with a poor prognosis.

In certain embodiments, the expression level of RNF167 can be used as a marker for determining the prognosis of a subject. In certain embodiments, the expression level of CASTOR1 can be used as a marker for determining the prognosis of a subject. In certain non-limiting embodiments, phosphorylation and/or ubiquitination of CASTOR1 can be used as a marker for determining the prognosis of a subject.

In certain embodiments, a prognostic method of the present disclosure includes determining the expression level of RNF167 in a cancer, e.g., in a sample of a cancer, of a subject. In certain embodiments, the method can further include comparing the expression level of RNF167 in the cancer to a reference control level of RNF167, where increased expression of RNF167 in the cancer compared to the RNF167 reference control level indicates that the subject has a poor prognosis. A “reference control level” or “reference control expression level” of RNF167, as used interchangeably herein, can, for example, be established using a reference control sample. Non-limiting examples of reference control samples include normal and/or healthy cells, e.g., from a sample of normal or benign tissue, that have wild-type RNF167 activity. In certain embodiments, a reference control level of RNF167 can, for example, be established using normal cells, e.g., benign cells, located adjacent to the tumor in a patient. In certain embodiments, the expression level of RNF167 can be the nucleic acid expression level of RNF167. Alternatively and/or additionally, the expression level of RNF167 can be the protein expression level of RNF167.

In certain embodiments, a prognostic method of the present disclosure includes determining the expression level of CASTOR1 in a cancer, e.g., a sample of a cancer, of a subject. In certain embodiments, the method can further include comparing the expression level of CASTOR1 in the cancer to a reference control level of CASTOR1, where reduced expression of CASTOR1 in the cancer compared to the CASTOR1 reference control level indicates that the subject has a poor prognosis. A “reference control level” or “reference control expression level” of CASTOR1, as used interchangeably herein, can, for example, be established using a reference control sample. Non-limiting examples of reference control samples include normal and/or healthy cells, e.g., from a sample of normal or benign tissue, that have wild-type CASTOR1 activity. In certain embodiments, a reference control level of CASTOR1 can, for example, be established using normal cells, e.g., benign cells, located adjacent to the tumor in a patient. In certain embodiments, the expression level of CASTOR1 can be the protein expression level of CASTOR1. Alternatively and/or additionally, the expression level of CASTOR1 can be the nucleic acid expression level of CASTOR1.

In certain embodiments, a prognostic method of the present disclosure includes determining the phosphorylation level and/or ubiquitination level of CASTOR1 in a cancer, e.g., a sample of a cancer, of a subject. In certain embodiments, the method can further include comparing the phosphorylation level and/or ubiquitination level of CASTOR1 in the cancer to a reference control level of CASTOR1, where increased phosphorylation level and/or ubiquitination level of CASTOR1 in the cancer compared to the CASTOR1 reference control level indicates that the subject has a poor prognosis. A “reference control level” or “reference control expression level” of CASTOR1, as used interchangeably herein, can, for example, be established using a reference control sample. Non-limiting examples of reference control samples include normal and/or healthy cells that have wild-type CASTOR1 activity. In certain embodiments, a reference control level of CASTOR1 phosphorylation and/or ubiquitination can, for example, be established using normal cells, e.g., benign cells, located adjacent to the tumor in a patient.

In certain embodiments, the phosphorylation level is the level of phosphorylation at a particular site. For example, but not by way of limitation, a prognostic method of the presently disclosed subject can include analyzing the level of CASTOR1 phosphorylation at a particular site, e.g., at a particular amino acid, e.g., serine 14 (S14). As described in the Example section, CASTOR1 phosphorylated at S14 is an indicator of mTORC1 activity, where increased S14 phosphorylation of CASTOR1 in the cancer compared to the CASTOR1 reference control level indicates that the subject has a poor prognosis.

In certain embodiments, the ubiquitination level is the ubiquitination level at a particular site. In certain embodiments, a prognostic method of the presently disclosed subject can include analyzing the level of CASTOR1 ubiquitination at one or more particular sites, e.g., at one or more amino acids, e.g., at one or more lysines (K), e.g., K61, K96 and/or K213.

Where comparisons to reference control expression levels are referred to herein, the expression level of a nucleic acid and/or protein of interest is assessed relative to the reference control expression level within the same species. For example, a human RNF167 expression level and/or presence are compared with a human RNF167 reference control level.

In certain embodiments, the absence and/or a reduced expression of CASTOR1 means the detection of less than about 90%, less than about 80%, less than about 70%, less than about 60%, less than about 50/%, less than about 40% or less than about 30% expression relative to the CASTOR1 reference control level. In certain embodiments, the increased expression of RNF167 means the detection of greater than about a 1.5-fold increase, greater than about a 2-fold increase, greater than about a 2.5-fold increase, greater than about 3-fold increase or greater than about a 3.5-fold increase in expression relative to the reference control level. In certain embodiments, the increased phosphorylation, e.g., at S14, and/or ubiquitination, e.g., at K61, K96 and/or K213, of CASTOR1 means the detection of greater than about a 1.5-fold increase, greater than about a 2-fold increase, greater than about a 2.5-fold increase, greater than about 3-fold increase or greater than about a 3.5-fold increase in phosphorylation and/or ubiquitination relative to the reference control level.

Methods for qualitatively and quantitatively detecting and/or determining the expression level of a nucleic acid, e.g., a RNF167 nucleic acid and/or a CASTOR1 nucleic acid, include, but are not limited to polymerase chain reaction (PCR), including conventional, qPCR and digital PCR, in situ hybridization (for example, but not limited to Fluorescent In situ Hybridization (“FISH”)), gel electrophoresis, sequencing and sequence analysis, microarray analysis and other techniques known in the art.

In certain embodiments, the method of detection can be real-time PCR (RT-PCR), quantitative PCR, fluorescent PCR, RT-MSP (RT methylation specific polymerase chain reaction), PicoGreen™ (Molecular Probes, Eugene, Oreg.) detection of DNA, radioimmunoassay or direct radio-labeling of DNA. For example, but not by way of limitation, a nucleic acid can be reversed transcribed into cDNA followed by polymerase chain reaction (RT-PCR); or, a single enzyme can be used for both steps as described in U.S. Pat. No. 5,322,770, or the nucleic acid can be reversed transcribed into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels. The levels of a mRNA of interest and a control mRNA can be quantitated in cancer tissue or cells and adjacent benign tissues.

In certain embodiments, the method of detection of the present disclosure can be carried out without relying on amplification, e.g., without generating any copy or duplication of a target sequence, without involvement of any polymerase, or without the need for any thermal cycling. In certain embodiments, detection can be performed using the principles set forth in the QuantiGene™ method described in U.S. application Ser. No. 11/471,025, filed Jun. 19, 2006, and is incorporated herein by reference.

In certain embodiments, in situ hybridization visualization can be employed, where a radioactively labeled antisense RNA probe is hybridized with a thin section of a biological sample, e.g., a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

In certain non-limiting embodiments, evaluation of nucleic acid expression can be performed by fluorescent in situ hybridization (FISH). FISH is a technique that can directly identify a specific region of DNA or RNA in a cell and therefore enables visual determination of the biomarker expression in tissue samples. The FISH method has the advantages of a more objective scoring system and the presence of a built-in internal control consisting of the biomarker gene signals present in all non-neoplastic cells in the same sample. FISH is a direct in situ technique that can be relatively rapid and sensitive, and can also be automated. Immunohistochemistry can be combined with a FISH method when the expression level of the biomarker is difficult to determine by FISH alone.

In certain embodiments, the expression of a nucleic acid can be detected on a DNA array, chip or a microarray. Oligonucleotides corresponding to the nucleic acids of interest, e.g., a RNF167 nucleic acid and/or a CASTOR1 nucleic acid, are immobilized on a chip which is then hybridized with labeled nucleic acids of a biological sample, e.g., tumor sample, obtained from a subject. Positive hybridization signal is obtained with the sample containing the nucleic acids of interest. Methods of preparing DNA arrays and their use are well known in the art. (See, for example, U.S. Pat. Nos. 6,618,679; 6,379,897; 6,664,377; 6,451,536; 6,548,257; U.S. Patent Application Nos. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application No. 20030215858).

In certain embodiments, to monitor a nucleic acid, e.g., RNF167 nucleic acid, expression levels, nucleic acid, e.g., mRNA, can be extracted from the biological sample to be tested, reverse transcribed and fluorescent-labeled cDNA probes can be generated. The labeled cDNA probes can then be applied to microarrays capable of hybridizing to a biomarker, allowing hybridization of the probe to microarray and scanning the slides to measure fluorescence intensity. This intensity correlates with the hybridization intensity and expression levels of the biomarker.

Types of probes for detection of nucleic acids include cDNA, riboprobes, synthetic oligonucleotides and genomic probes. The type of probe used will generally be dictated by the particular situation, such as riboprobes for in situ hybridization, and cDNA for Northern blotting, for example. In certain non-limiting embodiments, the probe is directed to nucleotide regions unique to the particular RNA. The probes can be as short as is required to differentially recognize the particular biomarker mRNA transcripts, and can be as short as, for example, 15 bases. Probes of at least 17 bases, 18 bases and 20 bases can also be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, ³²P and ³⁵S, or fluorophores. Labeling with radioisotopes can be achieved, whether the probe is synthesized chemically or biologically, by the use of suitably labeled bases.

Methods for detecting and/or determining the expression level of a protein, e.g., a RNF167 protein and/or a CASTOR1 protein and/or the phosphorylation and/or ubiquitination level of a CASTOR1 protein are well known to those skilled in the art, and include, but are not limited to, mass spectrometry techniques, I-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see, for example, U.S. Patent Application Nos. 2003/0013208; 2002/0155493, 2003/0017515 and U.S. Pat. Nos. 6,329,209 and 6,365,418, herein incorporated by reference in their entireties.

In certain non-limiting embodiments, a detection method for measuring protein expression and/or phosphorylation and/or ubiquitination includes the steps of contacting a biological sample, e.g., a tissue sample, with an antibody or variant (e.g., fragment) thereof, which selectively binds the biomarker, and detecting whether the antibody or variant thereof is bound to the sample. The method can further include contacting the sample with a second antibody, e.g., a labeled antibody. The method can further include one or more washing steps, e.g., to remove one or more reagents. In certain embodiments, the detection method can include contacting the sample with an antibody or variant (e.g., fragment) thereof that selectively binds to a phosphorylation and/or ubiquitination site. For example, but not by way of limitation, an antibody or variant (e.g., fragment) thereof for use in the methods disclosed herein can specifically bind to the S14 phosphorylation site of CASTOR1. In certain embodiments, an antibody or variant (e.g., fragment) thereof for use in the methods disclosed herein can specifically bind to one or more CASTOR1 ubiquitination sites, e.g., K61, K96 and/or K213.

In certain non-limiting embodiments, Western blotting can be used for detecting and quantitating protein expression levels. Cells can be homogenized in lysis buffer to form a lysate and then subjected to SDS-PAGE and blotting to a membrane, such as a nitrocellulose filter. Antibodies (unlabeled) can then brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.).

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay can be scored visually, using microscopy, and the results can be quantitated. Machine-based or autoimaging systems can also be used to measure immunostaining results for the biomarker.

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Labeled antibodies against proteins, e.g., an RNF167 protein and/or a CASTOR1 protein (or a phosphorylated CASTOR1 protein), can also be used for imaging purposes, for example, to detect the presence of the protein in cells of a subject. Suitable labels include radioisotopes, iodine (¹²⁵I, ¹²¹I), carbon (¹⁴C), sulphur (³⁵S), tritium (H), indium (¹¹²In), and technetium (99mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red. The labeled antibody or antibody fragment will preferentially accumulate at the location of cells which contain a biomarker. The labeled antibody or variant thereof, e.g., antibody fragment, can then be detected using known techniques.

In certain non-limiting embodiments, agents that specifically bind to a protein other than antibodies are used, such as peptides. Peptides that specifically bind can be identified by any means known in the art, e.g., peptide phage display libraries. Generally, an agent that is capable of detecting a protein, such that the presence of the protein is detected and/or quantitated, can be used.

In addition, a protein can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos. 2003/0199001, 2003/0134304, 2003/0077616, which are herein incorporated by reference in their entireties.

Mass spectrometry methods are well known in the art and have been used to quantify and/or identify biomolecules, such as proteins (see, e.g., Li et al. (2000) Tibtech 1:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait at al., Science 262:89-92 (1993); Keough at al., Proc. Natl. Acad. Sci. USA. 96:7131-6 (1999); reviewed in Bergman, EXS 88:133-44 (2000).

Detection of the presence of a nucleic acid and/or protein will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis), to determine the relative amounts of a particular biomarker. Software programs such as the Biomarker Wizard program (Ciphergen Biosystems, Inc., Fremont, Calif) can be used to aid in analyzing mass spectra.

Additional methods for determining nucleic acid and/or protein expression in samples are described, for example, in U.S. Pat. Nos. 6,271,002; 6,218,122; 6,218,114; and 6,004,755; and in Wang et al, J. Clin. Oncol., 22(9): 1564-1671 (2004); and Schena et al., Science, 270:467-470 (1995); all of which are incorporated herein by reference in their entireties.

Methods of Treatment

The present disclosure relates to methods for preventing and/or treating a disease and/or disorder of a subject. In certain embodiments, the disease can be a cancer and/or tumor in the subject. In certain embodiments, the disease can be diabetes. In certain embodiments, the disease can be ageing. In certain embodiments, the disease can be a disease related to metabolism.

The present disclosure provides methods for preventing and/or treating a disease, e.g., a cancer or tumor, in a subject by reducing the expression, activity and/or function of RNF167 and/or decreasing the degradation and/or phosphorylation and/or ubiquitination of CASTOR1 to increase the expression, activity and/or function of CASTOR1. In certain embodiments, methods for preventing and/or treating a disease, e.g., a cancer, a tumor, diabetes and/or ageing, in a subject include reducing the expression, activity and/or function of RNF167. In certain embodiments, methods for preventing and/or treating a disease, e.g., a cancer, a tumor, diabetes and/or ageing, in a subject include decreasing the degradation and/or phosphorylation and/or ubiquitination of CASTOR1 to increase the expression, activity and/or function of CASTOR1.

In certain non-limiting embodiments, the present disclosure provides for preventing and/or treating a subject that has a disease, e.g., a cancer or tumor. For example, but not by way of limitation, the method can include administering a therapeutically effective amount of an RNF167 inhibitor to the subject. In certain embodiments, administration of the RNF167 inhibitor inhibits the proliferation and/or survival of cancer cells in the subject.

Alternatively or additionally, a method of the present disclosure can include administering a therapeutically effective amount of an inhibitor to the subject that inhibits and/or reduces phosphorylation of CASTOR1, e.g., phosphorylation of CASTOR1 at amino acid S14, and/or inhibits or reduces the degradation of CASTOR1 and/or inhibits or reduces the ubiquitination of CASTOR1. In certain embodiments, a method of the present disclosure can include administering a therapeutically effective amount of a CASTOR1 agonist to the subject that increases and/or enhances the activity, function and/or expression of CASTOR1. In certain embodiments, administration of such an inhibitor and/or agonist inhibits the proliferation and/or survival of cancer cells in the subject.

In certain embodiments, the present disclosure provides a method for lengthening the period of survival of a subject having a disease, e.g., a cancer or tumor. For example, but not by way of limitation, one or more of the disclosed inhibitors and/or agonists, e.g., an RNF167 inhibitor, can be administered to a subject and prolong the survival of the subject relative to a control subject or control subject population not receiving the disclosed treatment. In certain embodiments, the period of survival is extended at least about 10 percent, at least about 25 percent, at least about 30 percent, at least about 50 percent, at least about 60 percent or at least about 70 percent. In certain embodiments, the period of survival is extended by about 1 month, about 2 months, about 4 months, about 6 months, about 8 months, about 10 months, about 12 months, about 14 months, about 18 months, about 20 months, about 2 years, about 3 years, about 5 years or more. In certain embodiments, the disclosed inhibitors can prolong the remission of a cancer in the subject relative to a control subject or control subject population not receiving the disclosed treatment.

In certain embodiments, the present disclosure provides methods for producing an anti-cancer effect in a subject. For example, but not by way of limitation, the method for producing an anti-cancer effect includes administering to a subject having a cancer and/or tumor a therapeutically effective amount of one or more of the disclosed inhibitors and/or agonists, e.g., an RNF167 inhibitor, to produce an anti-cancer effect in the subject. In certain embodiments, the anti-cancer effect is selected from the group consisting of a reduction in aggregate cancer cell mass, a reduction in cancer cell growth rate, a reduction in cancer cell proliferation, a reduction in tumor mass, a reduction in tumor volume, a reduction in cancer cell proliferation, a reduction in cancer growth rate, a reduction in cancer metastasis, and combinations thereof. In certain embodiments, the anti-cancer effect is a reduction in the number of cancer cells. In certain embodiments, the anti-cancer effect is a reduction in tumor size and/or a reduction in the rate of tumor growth. In certain embodiments, the anti-cancer effect is a reduction in the aggregate cancer cell burden. In certain embodiments, the anti-cancer effect is a reduction in the rate of cell proliferation and/or an increase in the rate of cell death. In certain embodiments, the anti-cancer effect is a prolongation of survival of the subject. In certain embodiments, the anti-cancer effect is a prolongation in the interval until relapse relative to a control subject or control subject population not receiving the disclosed treatment.

In certain embodiments, the present disclosure provides methods for reducing tumor growth in a subject. For example, but not by way of limitation, the method for reducing tumor growth includes administering to a subject a therapeutically effective amount of one or more of the disclosed inhibitors and/or agonists. In certain embodiments, tumor growth can be inhibited or reduced by decreasing expression, activity or function of RNF167. In certain embodiments, tumor growth can be inhibited or reduced by decreasing the phosphorylation and/or ubiquitination of CASTOR1 in the tumor cells. As shown in the Examples described herein, CASTOR1 can be a tumor suppressor in various cancers and tumors.

Methods disclosed herein can be used for treating any suitable cancers. Non-limiting examples of cancers that can be treated accordingly the presently disclosed methods include carcinomas, e.g., adenocarcinoma, lymphoma, blastoma, melanoma, sarcoma, and leukemia, melanoma, lung cancer, head and neck cancer, renal cell cancer, colon cancer, colorectal cancer, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer, bladder cancer, breast cancer, endometrial carcinoma, myeloma, salivary gland carcinoma, kidney cancer such as renal cell carcinoma and Wilms' tumors, basal cell carcinoma, prostate cancer, vulval cancer, thyroid cancer, testicular cancer, fallopian tube adenocarcinoma, and esophageal cancer. In certain embodiments, the cancer is breast cancer. In certain embodiments, the cancer can be a cancer and/or tumor in which mTORC1 is dysregulated. In certain embodiments, the cancer can be breast invasive carcinoma, brain lower grade glioma, skin cutaneous melanoma, head and neck squamous cell carcinoma, cervical squamous cell carcinoma and endocervical adenocarcinoma, lung adenocarcinoma, liver hepatocellular carcinoma, pancreatic adenocarcinoma, glioblastoma multiforme or acute myeloid leukemia. In certain embodiments, the cancer is lung cancer, a bladder carcinoma, a fibroadenoma, a breast cancer, a fallopian tube adenocarcinoma, a gall bladder adenocarcinoma, a kidney tumor, a pancreas adenocarcinoma, a skin malignant melanoma, a testis seminoma, a thyroid adenoma, and combinations thereof.

In certain embodiments, a method of the present disclosure can further include assessing the expression level of mTORC1 (or a subunit thereof, e.g., mTOR) in a cancer and/or tumor or a sample thereof. In certain embodiments, the sample can be obtained from the cancer and/or tumor of the subject. In certain embodiments, a method for treating a subject having a cancer can include determining the expression level of mTORC1 (or a subunit thereof) in a sample of the cancer, where if the expression level of mTORC1 (or a subunit thereof) is increased compared to an mTORC1 control level, then administering to the subject a therapeutically effective amount of an inhibitor disclosed herein, e.g., an RNF167 inhibitor. Any suitable detecting methods known in the art and disclosed herein can be used with the presently disclosed subject matter to detect the expression level of mTORC1 (or a subunit thereof) (e.g., at the nucleic acid or protein level) in a cancer and/or tissue.

In certain embodiments, a method of the present disclosure can further include assessing the expression level of RNF167 in a cancer and/or tumor or a sample thereof. In certain embodiments, the sample can be obtained from the cancer and/or tumor of the subject. In certain embodiments, a method for treating a subject having a cancer can include determining the expression level of RNF167 in a sample of the cancer, where if the expression level of RNF167 is increased compared to an RNF167 control level, then administering to the subject a therapeutically effective amount of an inhibitor disclosed herein, e.g., an RNF167 inhibitor and/or an inhibitor that inhibits and/or reduces phosphorylation of CASTOR1, e.g., phosphorylation of CASTOR1 at amino acid S14, and/or inhibits or reduces the degradation of CASTOR1. Any suitable detecting methods known in the art and disclosed herein can be used with the presently disclosed subject matter to detect the expression level of RNF167 (e.g., at the nucleic acid or protein level) in a cancer and/or tissue.

In certain embodiments, a method of the present disclosure can further include assessing the expression level of CASTOR1 in a cancer and/or tumor or a sample thereof. In certain embodiments, a method of the present disclosure can further include assessing the level of phosphorylated CASTOR1 (e.g., CASTOR1 phosphorylated at S14) in a cancer and/or tumor or a sample thereof. In certain embodiments, the sample can be obtained from the cancer and/or tumor of the subject. In certain embodiments, a method for treating a subject having a cancer can include determining the expression level of CASTOR1 in a sample of the cancer, where if the expression level of CASTOR1 is reduced and/or decreased compared to an CASTOR1 control level, then administering to the subject a therapeutically effective amount of an inhibitor disclosed herein, e.g., an RNF167 inhibitor and/or an inhibitor that inhibits and/or reduces phosphorylation of CASTOR1, e.g., phosphorylation of CASTOR1 at amino acid S14, and/or inhibits or reduces the degradation of CASTOR1. Any suitable detecting methods known in the art and disclosed herein can be used with the presently disclosed subject matter to detect the expression level of CASTOR1 (e.g., at the nucleic acid or protein level) or level of phosphorylated CASTOR1 in a cancer and/or tissue.

In certain embodiments, an inhibitor of the present disclosure can be administered to a subject at a dose of about 0.05 mg/kg to about 100 mg/kg. In certain embodiments, a subject can be administered up to about 2,000 mg of an inhibitor of the present disclosure in a single dose or as a total daily dose.

In certain embodiments, the dosage administered varies depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. In addition, it is to be understood that, for any particular subject, specific dosage regimes should be adjusted over time according to the individual need and the professional judgment of the person administering or supervising the administration of the inhibitor. For example, the dosage of the inhibitor can be increased if the lower dose does not provide sufficient activity in the treatment of a disease or condition described herein (e.g., cancer). Alternatively, the dosage of the inhibitor can be decreased if the disease (e.g., cancer) is reduced, no longer detectable or eliminated.

In certain embodiments, the disclosed inhibitors can be administered to the subject in a single dose or divided doses. In certain embodiments, the disclosed inhibitors can be administered to the subject once a day, twice a day, once a week, twice a week, three times a week, four times a week, five times a week, six times a week, once every two weeks, once a month, twice a month, once every other month or once every third month.

In certain embodiments, the duration of the disclosed treatment can be between about one week to about two years. In certain embodiments, the duration of the disclosed treatment is at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months. In certain embodiments, the duration of the disclosed treatment is at most about 1 week, at most about 2 weeks, at most about 3 weeks, at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 12 months, at most about 13 months, at most about 14 months, at most about 15 months, at most about 16 months, at most about 17 months, at most about 18 months, at most about 19 months, at most about 20 months, at most about 21 months, at most about 22 months, at most about 23 months, or at most about 24 months. In certain embodiments, the duration of the disclosed inhibitor treatment is at most 24 months or 2 years. In certain embodiments, the inhibitor can be administered until the cancer, cancer cells and/or tumor is no longer detectable.

In certain embodiments, the disclosed inhibitors can be cyclically administered to a subject. Cycling therapy involves the administration of the inhibitors for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment. In certain embodiments, the treatment stops after one cycle because the subject is intolerable to the adverse effects and toxicities associated with the disclosed inhibitors.

In certain embodiments, the number of cycles is from about one to about twenty-four cycles. In certain embodiments, the number of cycles is more than twenty-four cycles. In certain embodiments, the number of cycles is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24. In certain embodiments, the duration of a cycle is from about 21 to about 30 days. In certain embodiments, the duration of a cycle is about 21, about 22, about 23, about 24, about 25, about 26, about 27, about 28, about 29, or about 30 days. In certain embodiments, the duration of a cycle is about 27 days, about 28 days, about 29, or about 30 days. In certain embodiments, the number of cycles is about twenty-four cycles.

In certain embodiments, each cycle is followed by a rest period, where the disclosed inhibitors are not administered to the subject. In certain embodiments, the rest period is from about two weeks to about six weeks, from three weeks to about five weeks, from about four weeks to about six weeks. In certain embodiments, the rest period is about three weeks, about four weeks, about five weeks, or about six weeks. The present disclosure further allows the frequency, number, and length of dosing cycles and rest periods to be adjusted.

In certain embodiments, the inhibitors and agonists disclosed herein can be used alone or in combination with one or more agents, e.g., anti-cancer agents. For example, but not by way of limitation, methods of the present disclosure can include administering one or more inhibitors and one or more agents, e.g., anti-cancer agents. “In combination with,” as used herein, means that the inhibitor or agonist, e.g., RNF167 inhibitor, and the one or more agents, e.g., anti-cancer agents, are administered to a subject as part of a treatment regimen or plan. In certain embodiments, being used in combination does not require that the inhibitor or agonist and one or more agents, e.g., anti-cancer agents, are physically combined prior to administration, administered by the same route or that they be administered over the same time frame. In certain embodiments, the agent, e.g., anti-cancer agent, is administered before an inhibitor or agonist. In certain embodiments, the agent, e.g., anti-cancer agent, is administered after an inhibitor or agonist. In certain embodiments, the agent, e.g., anti-cancer agent, is administered simultaneously with an inhibitor or agonist. In certain embodiments, a CASTOR1 inhibitor or agonist disclosed herein can be administered in combination with one or more agents, e.g., anti-cancer agents.

In certain embodiments, the one or more agents can be an anti-cancer agent. Non-limiting exemplary anti-cancer agents include, but are not limited to, chemotherapeutic agents, radiotherapeutic agents, cytokines, anti-angiogenic agents, apoptosis-inducing agents, anti-cancer antibodies, a targeted drug, and/or agents which promote the activity of the immune system, including but not limited to cytokines such as but not limited to interleukin 2, interferon, an anti-CTLA4 antibody, an anti-PD-1 antibody and/or an anti-PD-L1 antibody, and checkpoint inhibitors. In certain embodiments, the anti-cancer agent can be a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, methotrexate, capecitabine, lapatinib, 5-FU, vincristine, etoposide or any combination thereof. In certain embodiments, the anti-cancer agent can be radiation therapy. Other non-limiting exemplary anti-cancer agents that can be used with the presently disclosed subject matter include tumor-antigen based vaccines and chimeric antigen receptor T-cells.

In certain embodiments, the one or more agents can be an agent that is the standard of care for a disease. For example, but not by way of limitation, the one or more agents can be the standard of care for treating diabetes, e.g., insulin, an insulin analog, metformin or similar diabetes agents, sulfonylureas, meglitinides, thiazolidinediones, DPP-4 inhibitors, GLP-1 receptor agonists and SGLT2 inhibitors.

In certain embodiments, the method can further include administering to the subject a therapeutically effective amount of an additional inhibitor selected from the group consisting of an AKT inhibitor, a TSC2 inhibitor, an mTORC1 inhibitor, e.g., an mTOR inhibitor, and combinations thereof. Non-limiting examples of such inhibitors can include a compound, a small molecule, a chemical, a polypeptide, a peptide, a protein, and combinations thereof that reduces the expression, function and/or activity of AKT, TSC2, and/or mTORC1. In certain embodiments, the additional inhibitor can be a ribozyme, an antisense oligonucleotide, a shRNA molecule and/or an siRNA molecule that reduces the expression, function and/or activity of AKT, TSC2, and/or mTORC1 (or a subunit thereof). Any suitable methods known in the art can be used for administering to the subject the disclosed inhibitors disclosed herein. In certain embodiments, the disclosed inhibitors can be administered to the subject orally or parenterally. For example, and not by way of limitation, the route of administration can be intravenous, intraarterial, intrathecal, intraperitoneal, intramuscular, subcutaneous, topical, intradermal, intranasal, vaginal, rectal, route, locally to the cancer, or combinations thereof.

### IV. Pharmaceutical Compositions

The present disclosure further provides pharmaceutical compositions comprising one or more inhibitors and/or agonist disclosed herein for use in treating a disease in a subject. In certain embodiments, the disease cancer and/or a tumor, diabetes and/or ageing. For example, but not by way of limitation, the inhibitor can be selected from the group consisting of a RNF167 inhibitor, an inhibitor that inhibits and/or reduces phosphorylation of CASTOR1, e.g., at S14, or inhibits and/or reduces degradation of CASTOR1, and a combination thereof. In certain embodiments, the present disclosure provides a pharmaceutical composition that includes an RNF167 inhibitor. In certain embodiments, the present disclosure provides a pharmaceutical composition that includes a CASTOR1 agonist.

In certain embodiments, a pharmaceutical composition of the present disclosure includes an inhibitor or agonist, e.g., an RNF167 inhibitor or a CASTOR1 agonist, and a pharmaceutically acceptable carrier. Suitable pharmaceutically acceptable carriers that can be used with the presently disclosed subject matter have the characteristics of not interfering with the effectiveness of the biological activity of the active ingredients, e.g., disclosed inhibitors/anti-cancer agents, and that is not toxic to the patient to whom it is administered. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers include gels, bioabsorbable matrix materials, implantation elements containing the inhibitor and/or any other suitable vehicle, delivery or dispensing means or material. Such pharmaceutically acceptable carriers can be formulated by conventional methods and can be administered to the subject. In certain embodiments, the pharmaceutical acceptable carriers can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, the suitable pharmaceutically acceptable carriers can include one or more of water, saline, phosphate buffered saline, dextrose, glycerol, ethanol or combinations thereof.

In certain non-limiting embodiments, the pharmaceutical compositions of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical composition is formulated as a capsule. In certain embodiments, the pharmaceutical composition can be a solid dosage form. In certain embodiments, the tablet can be an immediate release tablet. Alternatively or additionally, the tablet can be an extended or controlled release tablet. In certain embodiments, the solid dosage can include both an immediate release portion and an extended or controlled release portion.

In certain embodiments, the pharmaceutical compositions of the present disclosure can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, pharmaceutical compositions of the present disclosure can be administered to the patient intravenously in a pharmaceutically acceptable carrier such as physiological saline. In certain embodiments, the present disclosure provides a parenteral pharmaceutical composition comprising inhibitors disclosed herein.

In certain embodiments, the pharmaceutical compositions suitable for use in the presently disclosed subject matter can include compositions where the active ingredients, e.g., an RNF167 inhibitor, are contained in a therapeutically effective amount. The therapeutically effective amount of an active ingredient can vary depending on the active ingredient, compositions used, the cancer and its severity, and the age, weight, etc., of the subject to be treated. In certain embodiments, a subject can receive a therapeutically effective amount of the disclosed inhibitors in single or multiple administrations of one or more composition, which can depend on the dosage and frequency as required and tolerated by the patient.

In certain non-limiting embodiments, pharmaceutical compositions of the present disclosure can include one or more additional inhibitors or be administered in combination with one or more additional inhibitors. For example, but not by way of limitation, the additional inhibitor can include a protein kinase B (AKT) inhibitor, a tuberous Sclerosis Complex 2 (TSC2) inhibitor, an mTORC1 (or a subunit of mTORC1) inhibitor, or a combination thereof. In certain non-limiting embodiments, an inhibitor that can regulate cell proliferation can be used in combination with the disclosed subject matter. For example, but not by way of limitation, any inhibitors that can target any kinases that mediate cell proliferation can be used in combination with the disclosed subject matter. Non-limiting examples of kinase inhibitors include afatinib, alectinib, axitinib, bevacizumab, bosutinib, cetuximab, crizotinib, canertinib, dasatinib, erlotinib, fostamatinib, gefitinib, GSK1838705A, ibrutinib, imatinib, lapatinib, lenvatinib, mubritinib, nilotinib, panitumumab, pazopanib, pegaptanib, ranibizumab, ruxolitinib, sorafenib, sunitinib, su6656, trastuzumab, tofacitinib, vandetanib and vemurafenib.

In certain embodiments, pharmaceutical compositions of the present disclosure can include or more anti-cancer agents or administered in combination with one or more additional anti-cancer agents. Non-limiting exemplary anti-cancer agents include, but are not limited to, chemotherapeutic agents, radiotherapeutic agents, cytokines, anti-angiogenic agents, apoptosis-inducing agents, anti-cancer antibodies, a targeted drug, agents which promote the activity of the immune system, and checkpoint inhibitors. Other non-limiting exemplary anti-cancer agents that can be used with the presently disclosed subject matter include tumor-antigen based vaccines and chimeric antigen receptor T-cells.

### V. Kits

The present disclosure provides kits that can be used to practice the presently disclosed methods of treating a disease, e.g., a cancer and/or tumor, diabetes or ageing in a subject. In certain embodiments, the kits disclosed herein comprise a pharmaceutical composition disclosed herein.

In certain embodiments, the kits comprise a pharmaceutical composition comprising a therapeutically effective amount of an inhibitor or agonist disclosed herein, e.g., an RNF167 inhibitor; an inhibitor that reduces phosphorylation of CASTOR1, e.g., at S14, or inhibits and/or reduces degradation of CASTOR1; an agonist of CASTOR1 that increases and/or enhances the activity, function and/or expression of CASTOR1; or a combination thereof.

In certain embodiments, the kit can include one or more additional inhibitors within the same container (or pharmaceutical compositions thereof) or within a second container. For example, but not by way of limitation, the additional inhibitor can include a protein kinase B (AKT) inhibitor, a tuberous Sclerosis Complex 2 (TSC2) inhibitor, a mTORC1 inhibitor, or a combination thereof.

In certain embodiments, the kit can include one or more anti-cancer agents. Non-limiting exemplary anti-cancer agents include, but are not limited to, chemotherapeutic agents, radiotherapeutic agents, cytokines, anti-angiogenic agents, apoptosis-inducing agents, anti-cancer antibodies, a targeted drug, agents which promote the activity of the immune system, and checkpoint inhibitors. Other non-limiting exemplary anti-cancer agents that can be used with the presently disclosed subject matter include tumor-antigen based vaccines and chimeric antigen receptor T-cells.

In certain embodiments, the kits further comprise instructions for using the kits in treating a subject. In certain embodiments, the instructions indicate that the disclosed inhibitors can be administered in accordance with the methods disclosed herein. In certain embodiments, the instructions indicate the disclosed inhibitors can be administered for up to 2 years. For example, and not by way of limitation, the instructions can include a description of the disclosed inhibitors and/or agonists and/or a second anti-cancer agent, and, optionally, other components present in the kit. In certain embodiments, the instructions can describe methods for administration of the components of the kit, including methods for determining the proper state of the subject, the proper dosage amount and the proper administration method for administering one or more of the disclosed inhibitors and/or other anti-cancer agent. Instructions can also include guidance for monitoring the subject over the duration of the treatment time. In certain embodiments, the kit can further comprise one or more containers comprising additional inhibitors and/or other anti-cancer agents.

In certain non-limiting embodiments, the present disclosure provides for a kit of this disclosure further including one or more of the following: devices and additional reagents, and components, such as tubes, containers, cartridges, and syringes for performing the methods disclosed herein.

In certain embodiments, a kit of the present disclosure can include one or more agents for detecting the expression level of RNF167, CASTOR1 and/or mTORC1 (or a subunit thereof) in a sample from the subject. For example but not by way of limitation, a kit of the present disclosure can be used for determining the prognosis of a subject having cancer. In certain embodiments, a kit for determining the prognosis of a subject having cancer can include means for detecting ring finger protein 167 (RNF167) and/or CASTOR1 that include one or more comprises one or more primers, probes, arrays/microarray, antibodies and/or bead for detecting RNF167 and/or CASTOR1. In certain embodiments, the means for detecting CASTOR1 comprises an antibody that specifically binds to S14 of CASTOR1. In certain embodiments, a kit for determining the prognosis of a subject having cancer further includes (i) a therapeutically effective amount of a ring finger protein 167 (RNF167) inhibitor; (ii) a therapeutically effective amount of an inhibitor that reduces phosphorylation of CASTOR1 at S14 and/or reduces degradation and/or ubiquitination of CASTOR1; and/or (iii) a therapeutically effective amount of an agonist of CASTOR1 that increases and/or enhances the activity, function and/or expression of CASTOR1.

The following examples are merely illustrative of the presently disclosed subject matter and should not be considered as limiting in any way.

### Example 1: RNF167 Activates mTORC1 and Promotes Tumorigenesis by Targeting CASTOR1 for Ubiquitination and Degradation

(CASTOR1) functions as an arginine sensor and regulates mTORC1 activity in response to arginine status. CASTOR1 is expected to contain a consensus AKT1 phosphorylation motif R—V—R—V-L-S14 (SEQ ID NO: 26). Proteomic analysis identified CASTOR1 phosphorylation at S14, suggesting that CASTOR1 is a potential AKT1 substrate. Examination with point mutation prediction algorithms revealed increased stability of CASTOR1 if S14 is mutated to a non-phosphorylatable mimic alanine (A), and a decreased stability if it is mutated to a constitutively phosphorylated mimic aspartic acid (D). These analyses suggest that AKT1 might phosphorylate CASTOR1 and regulate its stability.

The phosphorylation-dependent regulation of protein stability is closely associated with protein polyubiquitination, a mark for their degradation via 26S proteasome. The formation of polyubiquitin chain conjugated to a target protein occurs in a cascade of three steps: activation, conjugation, and ligation, exerted by E1 ubiquitin-activating enzyme, E2-conjugating enzyme, and E3 ubiquitin-ligase, respectively. The first linkage is initiated by the binding of the C-terminal glycine in ubiquitin to the lysine in the substrate, forming an isopeptide bond. Further polyubiquitin chain can be formed by linking the glycine residue of another ubiquitin molecule to the lysine of ubiquitin bound to a substrate. Seven lysine residues in ubiquitin are responsible for polyubiquitin formation, including K6, K11, K27, K29, K33, K48, and K63. Among them, K29-, K48- or K63-mediated polyubiquitination typically triggers proteasomal degradation. RING finger protein (RNF167) is a RING-type E3 ligase involved in regulating protein trafficking, localization, and degradation by directly ubiquitinating targeted substrates.

This Example shows that a low expression level of CASTOR1 is correlated with poor patient survival in numerous types of cancer including breast cancer, and that CASTOR1 is a substrate of RNF167. Furthermore, AKT-mediated phosphorylation of CASTOR1 facilitates its interaction with RNF167, leading to CASTOR1 ubiquitination and proteasome-dependent degradation. Additionally, CASTOR1 phosphorylation at S14 by AKT decreases its binding affinity to the GATOR2 complex. The phosphorylation and degradation of CASTOR1 collectively release the GATOR2 complex, activate mTORC1, and promote breast cancer progression. These findings reveal a novel mechanism by which cancer cells overcome the suppressive effect of CASTOR1 in the nutrient-deficient tumor microenvironment, and hence identify a potential novel therapeutic target for treating mTORC1-associated diseases including cancer.

Methods:

Cell culture and transfection: 293T cells obtained from ATCC (CRL-3216) were maintained in DMEM supplemented with 10% FBS. MCF7, T47D, HCC1569, and HCC202 cells were obtained from Dr. Xiaosong Wang at the University of Pittsburgh, and cultured in RPMI1640 with 10% FBS. MM cells were cultured in DMEM plus 10% FBS, while KMM cells were cultured in DMEM plus 10% FBS and 10 μg/ml hygromycin. HSAEC (FC-0016) and HLBEC (FC-0054) cells were purchased from Lifeline and cultured with the BronchiaLife™ Epithelial Airway Medium Complete Kit (Lifeline LL-0023). All cells were maintained at 37 C° in 5% CO₂.

For amino acid deprivation and re-stimulation to assess mTORC1 activation, cells were incubated in EBSS medium (Thermo 24010043) for 50 min and then stimulated by adding arginine (Sigma A5131) at the indicated concentration. For ubiquitination assays, cells were deprived of FBS or arginine, or treated with 10 μM MK2206 (Selleckem S1078) overnight before immunoprecipitation and immunoblotting, or re-stimulated with FBS or arginine for 12 h before analysis.

For chemical treatments, CHX (CST 2112) or MG132 (Sigma M8699) dissolved in DMSO (VWR 97061-250) was diluted in medium to a specified concentration. Medium containing MG132 or CHX was then used to replace the original medium, and cells were cultured in the presence of MG132 for a specified time.

For transfection, Lipofectamine 2000 (Thermo 11668019) was used for transient transfection of plasmids, and RNAimax (Thermo 13778150) was used for transfection of siRNAs based on the manufacturer's instructions.

Plasmids: Plasmids purchased from Addgene included: pLKO1-TRC (10878), pcDNA3-myr-HA-AKT1 (46969), pcDNA3-HA-AKT1 (73408), pcDNA3-HA-AKT1-K179M (73409), pcDNA3-HA-AKT1-1-149aa (73410), pcDNA3-HA-AKT1-120-433aa (73411), pRK5-HA-Ubiquitin-WT (17608), pRK5-HA-Ubiquitin-K29 (22903) and pRK5-HA-Ubiquitin-K29R (17602). Plasmids p³0.3 empty vector, p3.3-Myc-Ubiquitin-WT, p3.3-Myc-Ubiquitin-K48, p3.3-Myc-Ubiquitin-K63, p3.3-flag-KLHL19, p3.3-flag-KLHL21, p3.3-flag-KLHL22, p3.3-flag-ZNRF1, p3.3-flag-ZNRF2, p3.3-flag-BACURD1, p3.3-flag-BACURD2, p3.3-flag-RNF152, p³0.3-flag-RNF167, p3.3-flag-β-Trcp1, p3.3-flag-FBW7, p3.3-flag-HERC5 and p3.3-flag-Skp2 were provided by Jie Chen at Beijing University in China. pcDNA3 empty vector was purchased from Invitrogen. pMD.G and p8.74 were from PlasmidFactory. Human pITA-flag-CASTOR1 WT was cloned from 293T cells. Rat pITA-flag-CASTOR1 WT was previously described. The mutants of human pITA-flag-CASTOR1 including S14A, S14D, K61R, K96R, K213R, K61R/K96R, K61R/K213R and K61R/K96R/K213R were generated using a mutagenesis kit (NEB E0554) based on the manufacturer's instructions. The primer sequences used for the cloning are listed in Table 1 and the sequences of all plasmids were confirmed by direct sequencing. The siRNAs used for the disclosed subject matter are listed in Table 2.

TABLE 1
Summary of PCR primers and shRNAs

| | |
|---|---|
| Rat | 5′TATGCGGCCGCGCCACCATG |
| Flag- | GACTACAAAGACGATGACGA |
| CAST | CAAGATGGAACTTCACATCC |
| OR1- | AGAGC3′ |
| forward | (SEQ ID NO: 1) |
| Rat | 5′ATAGGATCCCTATGGATCTT |
| Flag- | TGGAAGCCAGG3′ |
| CAST | (SEQ ID NO: 2) |
| OR1- | |
| reverse | |
| Human | 5′TATGCGGCCGCGCCACCATG |
| CAST | GAGCTGCACATCCTAGAAC3′ |
| OR1- | (SEQID NO: 3) |
| forward | |
| Human | 5′ATAGGATCCTCAGGAAGCCA |
| CAST | GGCCTTCCT3′ |
| OR1- | (SEQ ID NO: 4) |
| reverse | |
| Human | 5′TATGCGGCCGCGCCACCATG |
| HA- | TACCCATACGATGTTCCAGA |
| CAST | TTACGCTATGGAGCTGCACA |
| OR1- | TCCTAGAAC3′ |
| forward | (SEQ ID NO: 5) |
| Human | 5′ATAGGATCCTCAGGAAGCCA |
| HA- | GGCCTTCCT3′ |
| CAST | (SEQ ID NO: 6) |
| OR1- | |
| reverse | |
| Human | 5′TATGCGGCCGCGCCACCATG |
| Flag- | GACTACAAAGACGATGACGA |
| CAST | CAAGATGGAGCTGCACATCC |
| OR1- | TAGAAC3′ |
| forward | (SEQ ID NO: 7) |
| Human | 5′ATAGGATCCTCAGGAAGCCA |
| Flag- | GGCCTTCCT3′ |
| CAST | (SEQ ID NO: 8) |
| OR1- | |
| reverse | |
| Human | 5′GCGGGTGCTGGCTGTCGCCC |
| Flag- | GTC3′ |
| CAST | (SEQ ID NO: 9) |
| OR1- | |
| S14A- | |
| forward | |
| Human | 5′ACCCGGTGTTCTAGGATG3′ |
| Flag- | (SEQ ID NO: 10) |
| CAST | |
| OR1- | |
| S14A- | |
| reverse | |
| Human | 5′GCGGGTGCTGGATGTCGCCC |
| Flag- | GTC3′ |
| CAST | (SEQ ID NO: 11) |
| OR1- | |
| S14D- | |
| forward | |
| Human | 5′ACCCGGTGTTCTAGGATG3′ |
| Flag- | (SEQ ID NO: 12) |
| CAST | |
| OR1- | |
| S14D- | |
| reverse | |
| Human | 5′GGAGGGCTTTCGAGAGCTGC |
| Flag- | CCC3′ |
| CAST | (SEQ ID NO: 13) |
| OR1- | |
| K61R- | |
| forward | |
| Human | 5′TCGTCCACCATAAGCGTG3′ |
| Flag- | (SEQ ID NO: 14) |
| CAST | |
| OR1- | |
| K61R- | |
| reverse | |
| Human | 5′TGGGGTCACCCGGATCGCCC |
| Flag- | GTTCGG3′ |
| CAST | (SEQ ID NO: 15) |
| OR1- | |
| K96R- | |
| forward | |
| Human | 5′GCAGCCTGCACTGCCGCA3′ |
| Flag- | (SEQ ID NO: 16) |
| CAST | |
| OR1- | |
| K96R- | |
| reverse | |
| Human | 5′CAGCACCCCCCGGGAGGCAG |
| Flag- | CCT3′ |
| CAST | (SEQ ID NO: 17) |
| OR1- | |
| K213R- | |
| forward | |
| Human | 5′TGCGAGTAGAAGAGGACATC |
| Flag- | TATG3′ |
| CAST | (SEQ ID NO: 18) |
| OR1- | |
| K213R- | |
| reverse | |
| shRNA | 5′TTGTACTACACAAAAGTACT |
| non- | G3′ |
| targeting | (SEQ ID NO: 19) |
| (NT) | |
| control | |
| Human | 5′GGAGCTGCACATCCTAGAAC |
| CAST | A3′ |
| OR1- | (SEQ ID NO: 20) |
| sh1 | |
| Human | 5′GCTTTGATGAATGTGGCATC |
| CAST | G3′ |
| OR1- | (SEQ ID NO: 21) |
| sh2 | |

TABLE 2
Summary of siRNAs

| siRNA negative control (NC) | Sigma | Cat#SIC001 |
|---|---|---|
| Human AKT1 siRNA-1 | Sigma | Cat#SASI_Hs01_00105954 |
| Human AKT1 siRNA-2 | Sigma | Cat#SASI_Hs01_00105953 |
| Human RNF167 siRNA-1 | Sigma | Cat#SASI_Hs01_00201491 |
| Human RNF167 siRNA-2 | Sigma | Cat#SASI_Hs01_00201493 |
| Human TSC2 siRNA-1 | Sigma | Cat#SASI_Hs01_00127335 |
| Human TSC2 siRNA-2 | Sigma | Cat#SASI_Hs01_00127336 |

Antibodies: Primary antibodies included antibodies to S6K1 (Abcam 32359), pS6K-Thr389 (CST 9205), p4EBP1-Ser65 (CST 9451), 4EBP1 (CST 9644), pan AKT (Cell Signaling Technology 4691), pAKT-Thr308 (CST 2965), AKT1 (CST 2938), pAKT substrate (RXRXXpS*/T*) (CST 10001), GAPDH (CST 5174), flag (Sigma F1804), flag (Sigma A9594), HA (CST 3724), HA (CST 3444), GST (CST 2625), Ub (Santa Cruz sc-8017), c-Myc (Santa Cruz sc-40), RNF167 (Santa Cruz sc-515405), RNF167 (Proteintech 24618-1-AP), β-actin (Santa Cruz sc-47778) and β-tubulin (Sigma 7B9). Antibodies to CASTOR1 were described as before. Secondary antibodies included mouse anti-Rabbit IgG (Light-Chain Specific) (CST 93702), rabbit anti-Mouse IgG (Light Chain Specific) (CST 58802), goat anti-rabbit HRP conjugated IgG (CST 7074), horse anti-mouse IgG HRP conjugated IgG (CST 7076), goat anti-mouse IgG DyLight 800 (Bio-Rad STAR117D800GA) and goat anti-rabbit IgG StarBright Blue700 (Bio-Rad 12004161).

Immunoprecipitation. Cells were lysed in lysis buffer (50 mM Tris-HCl, with 150 mM NaCl, 1 mM EDTA, and 1% Triton X-100, pH 7.4) supplemented with a complete protease inhibitor cocktail (Thermo 78438) and phosphatase inhibitor (Thermo 78427), followed by centrifugation at 4° C. for 5 min. The supernatant was then precleared with mouse IgG agarose beads (Sigma A0919) at 4° C. for 4 h, and subsequently mixed with washed agarose beads conjugated with anti-Flag (Sigma A2220), anti-HA (Thermo 26182), anti-Myc (Sigma A7470, anti-AKT (Cell Signaling Technology 3653) or mouse IgG antibodies (Sigma A0919) at 4 C° overnight. Immunocomplexes were washed extensively 3 times with washing buffer (50 mM Tris-HCl, 150 mM NaCl, pH 7.4). The immunoprecipitates were eluted with 2×SDS, and then subjected to immunoblotting analysis.

For transfection experiments, 6×10⁸ cells were seeded in 10 cm dishes and transfected with 5 μg of each plasmid using Lipofectamine 2000 (Thermo 11668019) for 48 h. Cells were then treated and lysed as described above.

Immunoblotting analysis: To detect all proteins except CASTOR1, samples were separated with 4%-20% SDS-polyacrylamide gels (Genscript M00656 and M00657). To detect CASTOR1 protein, samples were resolved with 10% SDS-polyacrylamide gels (Genscript M00665 and M00666). Proteins resolved in gels were then transferred to nitrocellulose membranes (GE Healthcare 10600004), which were incubated with primary and secondary antibodies overnight and for 1 h at room temperature, respectively. The signals were developed using the Luminiata Crescendo Western HRP Substrate (EMD Millipore WBLUR0500) and SuperSignal West Femto Maximum Sensitivity Substrate (Thermo 34096) or fluorescence secondary antibodies. The images were recorded with a ChemiDoc MP Imaging System (Bio-Rad 17001402) at either Chemi, Dylight 500, DyLight 800, or StarBright B700 channels.

In vitro kinase assay: Recombinant GST-AKT1 protein (Novus Biologicals, 1775-KS) was mixed with GST-CASTOR1 protein (Novus Biologicals, H00652968-P01) in a 30 μl reaction mixture at room temperature for 1 h. The reaction mixture contained protease inhibitors, 100 mM HEPES (pH 7.4), 150 mM NaCl, 50 mM MgCl₂,1 mM DTT, 0.01% NaN₃, 1 mM ATP, 0.2 μg GST-AKT1 and 1 μg GST-CASTOR1.

Lentivirus-mediated overexpression and knockdown of genes: CASTOR1 shRNAs, non-targeting control (NT), Flag-tagged CASTOR1 WT, S14A, and S14D expression lentiviral plasmids or the empty vector control pITA was cotransfected with pMDG and p8.74 packaging plasmids into 293T cells using the Lipofectamine 2000 (Thermo 11668019). At day 2 and 3 post-transfection, the supernatant of 293T cells was collected and filtered with a 0.45 μM filter. The transduction of cells was done by spinning infection at 1,500 rpm at room temperature for 1 h with 10 μg/ml polybrene (Sigma A5431). The expression of CASTOR1 was confirmed by immunoblotting at day 3 post-transduction.

Colony formation in softagar: A total of 2×10⁴ MCF7 or HCC1569 cells were suspended in 1 ml of 0.3% top agar (Sigma A5431) and then plated onto one well of 0.5% base agar in 6 well plates, which were maintained for 10 or 30 days, respectively. Colonies were photographed with a 4×objective with an inverted microscope.

BrdUincorporation and apoptosis assay: For BrdU incorporation, MCF7 or HCC1569 cells were pulsed with 10 μM BrdU (Sigma B5002) for 2 h, and then fixed with 70% ethanol, permeabilized with 2 M hydrochloric acid and stained with an anti-BrdU monoclonal antibody (Thermo B35129). Apoptotic cells were detected by co-staining with DAPI (Sigma D9542) and PE-Cy7 Annexin V Apoptosis Detection kit (eBioscience 88810374) following the instructions of the manufacturer. Flow cytometry was performed in a BD LSRFortessa system (BD Biosciences) and the analysis was done with FlowJo.

Reverse transcription real-time quantitative polymerase-chain reaction (RT-qPCR): Total RNA was extracted by using TRI Reagent (Sigma T9424) based on the manufacturer's instructions. Total RNA was subjected to reverse transcription using the Maxima H Minus First Strand cDNA Synthesis Kit (Thermo K1652). SsoAdvanced™ Universal SYBR® Green Supermix Kit (Bio-Rad 172-5272) was applied for qPCR analysis. The relative mRNA levels were normalized to a house-keeping gene, which yielded 2^{-ΔΔCt} values. For qPCR reaction, each sample was run in triplicates with cycle threshold (Ct) values within 0.5 Ct differences among the triplicates. The primers used for gene expression were 5′GCCACCACCCTCATAGATGT3′ (forward; SEQ ID NO: 22) and 5′AGGAGGTCACTGGGGAACTT3′ (reverse; SEQ ID NO: 23) for human CASTOR1; and ATCATTGCTCCTCCTGAGCG (forward; SEQ ID NO: 24) and CGGACTCGTCATACTCCTGC (reverse; SEQ ID NO: 25) for human β-actin.

Mouse experiments: Athymic Nude-Foxn1nu mice were purchased from Envigen. Mice were raised under 12 h light/dark cycle and with a standard diet at the University of Pittsburgh. MCF7 cells transduced with either a vector control, Flag-CASTOR1 WT, S14A, or S14D were trypsinized and concentrated by centrifugation to 5×10⁶ per 100 μl in DMEM supplemented with 10% FBS. An equal volume of cells was mixed with an equal volume of Matrigel (VWR 47743-720), and then 5×10⁶ cells were subcutaneously injected into each flank of the mouse. The mice were inserted with an estrogen pellet (Sigma 8875) before injection. Tumor volume was measured twice a week and calculated based on the formula (V=L×W×W×0.5). Mice were euthanized when the tumor size reached the upper limit of 1,500 mm³.

Quantification, statistical analysis, and reproducibility: The intensity of a protein band was quantified with Image Lab Software (Bio-Rad). Data were presented as mean±SEM (standard error of the mean) and analyzed by Student's t-test or one-way analysis of variance (ANOVA) if multiple samples were involved, followed by Tukey post-hoc test if P<0.05. All statistical analyses were done with the Prism software package (PRISM 6.0, GraphPad Software, USA). A P<0.05 was considered as statistically significant. Statistical symbols “*”, “**” and “***” indicate P-values <0.05, <0.01 and <0.001, respectively, and “NS” denotes “not significant”.

### Results

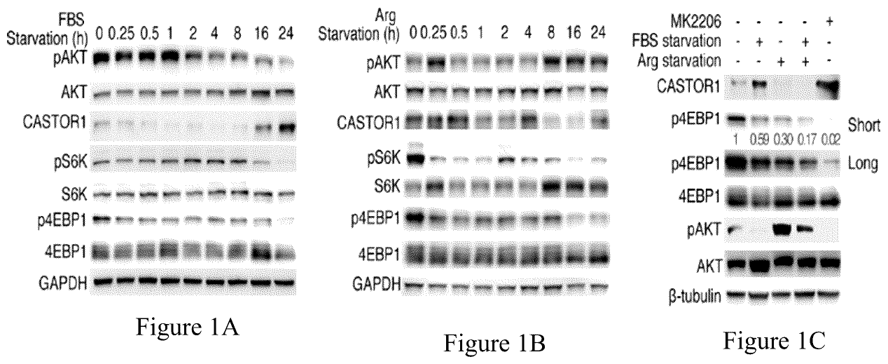

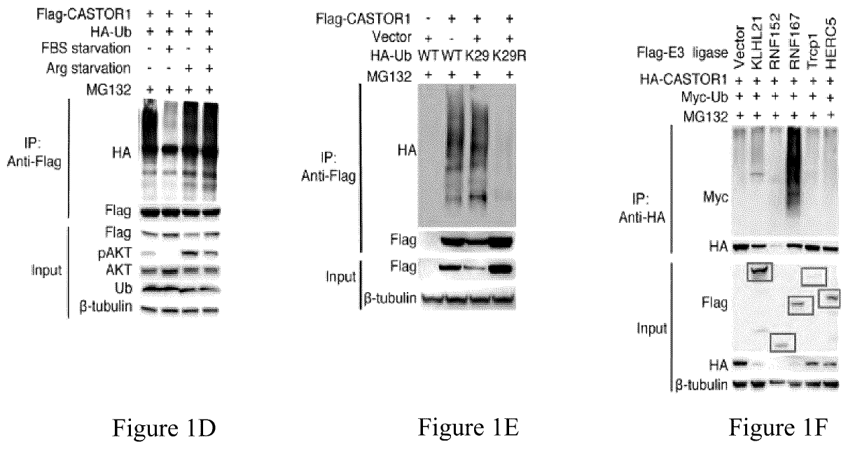

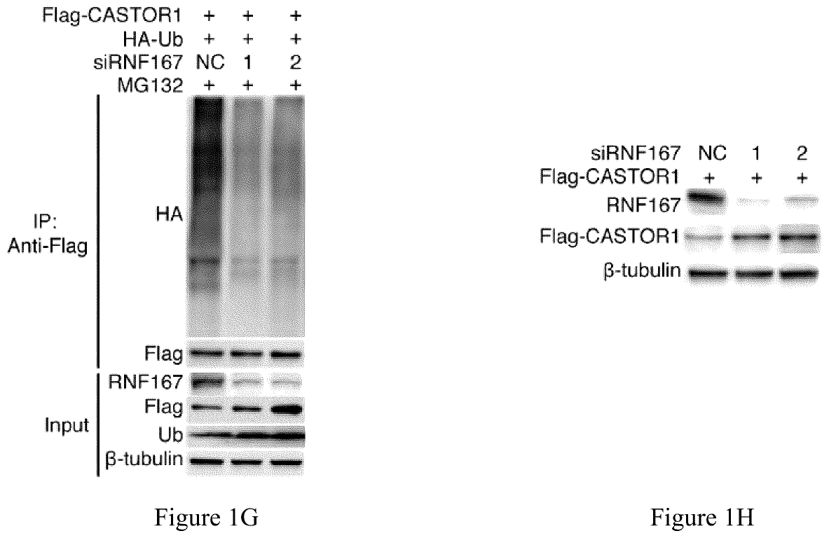

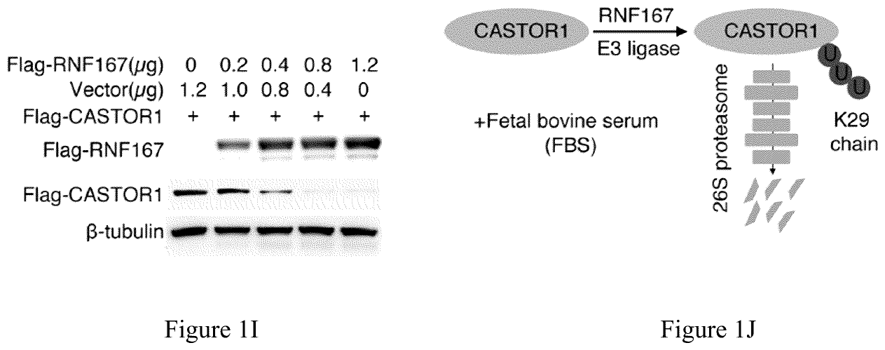

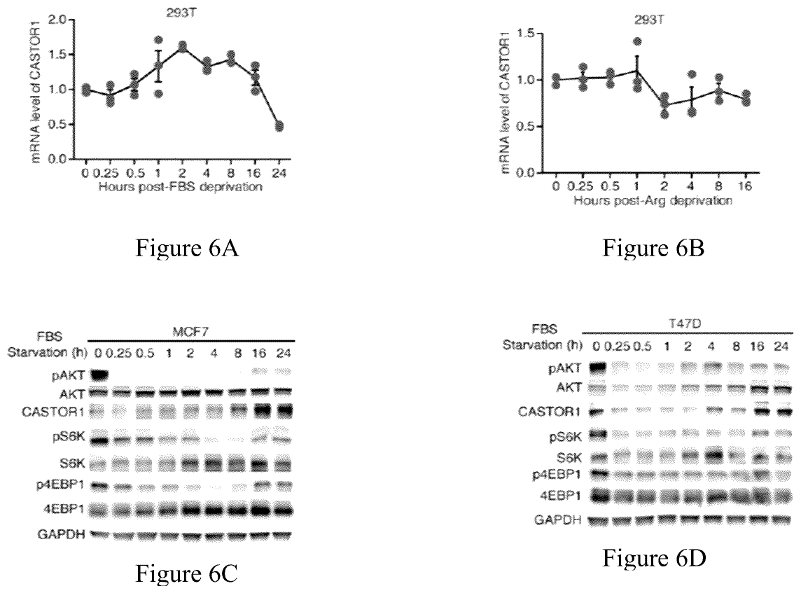

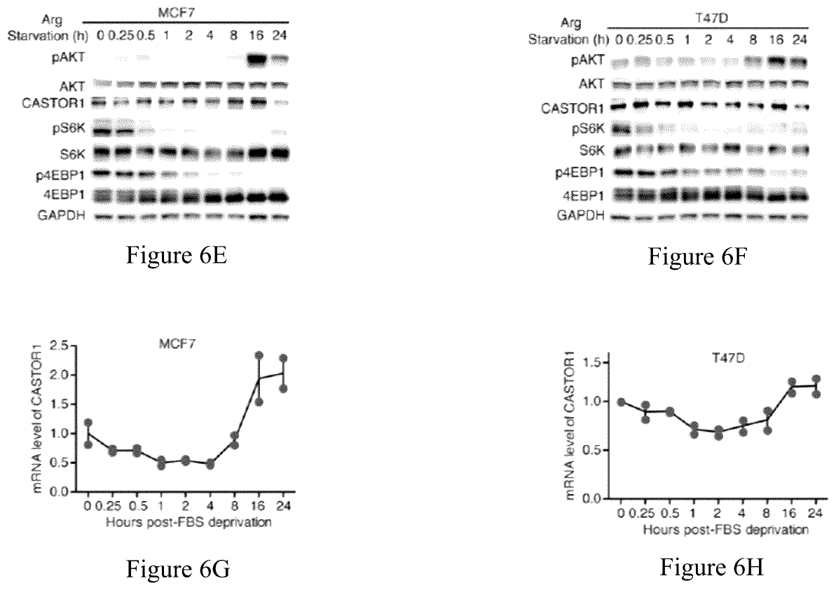

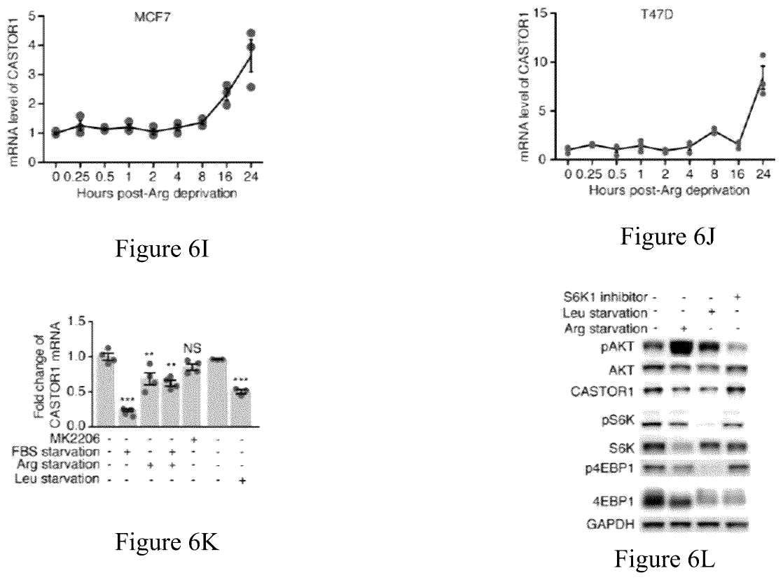

RNF167 mediates K29-linked polvubiquitination and degradation of CASTOR1 in response to growth factors: To reveal the environmental cue that activates mTORC1 by modulating the expression of CASTOR1, cells were deprived of either fetal bovine serum (FBS) or arginine. The kinetic analysis demonstrated that CASTOR1 protein level but not mRNA level was increased following 16 h of FBS deprivation in 293T cells, which was correlated with a decreased mTORC1 activity as shown by the reduced phosphorylation level of its downstream targets S6K and 4EBP1 (FIG. 1A, and FIG. 6A). As expected, the level of AKT activation was significantly reduced, which was noticeable at as early as 2 hrs but more obvious after 16 hrs following FBS deprivation (FIG. 1A). Hence, the level of CASTOR1 protein inversely trailed that of AKT activation following FBS deprivation. In contrast, arginine deprivation for as short as 15 min in 293T cells resulted in decreased mTORC1 activity (FIG. 1B). However, there were only marginal fluctuations of activated AKT and CASTOR1 protein levels before the first 4 hrs of arginine deprivation. The decreased mTORC1 activity was likely due to the released arginine inhibitory effect on CASTOR1. Extended arginine deprivation for more than 8 h enhanced AKT activation as a result of the feedback effect of mTORC1 inhibition, which was correlated with a slight decrease of CASTOR1 protein level as well as a slight decrease of mRNA level (FIG. 1B, and FIG. 6B). There was no increase of mTORC1 activity in these later time points despite the increased AKT activity and reduced CASTOR1 protein level. This was likely due to its already low mTORC1 activity as well as the requirement of arginine for its activation. In agreement with the results in 293T cells, deprivation of either FBS or arginine inactivated mTORC1 in ER+ breast cancer cell lines MCF7 and T47D, albeit their response kinetics varied (FIGS. 6C-6F). FBS deprivation inactivated AKT at as early as 15 min, and CASTOR1 protein level started to increase by 8 hrs following FBS deprivation (FIGS. 6C-6D). Thus, similar to 293T cells, the level of CASTOR1 protein inversely trailed that of AKT activation following FBS deprivation in these cells. Following arginine deprivation, marginal fluctuations of activated AKT and CASTOR1 protein levels were also observed within the first 1 hr (FIGS. 6E-6F). However, a decrease of activated AKT was observed between 2 and 4 hrs, which led to a slight increase of CASTOR1 protein level at 8 and 16 hrs. The mTORC1 activity was not further decreased at these time points, which was likely due to its already extremely low level. Similar to 293T cells, enhanced AKT activation was observed after 16 hr, a result of the feedback effect of mTORC1 inhibition, which led to a slight decrease of CASTOR1 protein level at 24 hr following arginine deprivation (FIGS. 6E-6F). Only a slight increase of mTORC1 activity was observed at this time point, which again indicated the essential role of arginine in mTORC1 activation. Intriguingly, FBS deprivation slightly increased while arginine deprivation dramatically increased the CASTOR1 mRNA level in these cells (FIG. 6G-6J).

The above results showed a negative correlation of AKT activation with the CASTOR1 protein level, which was strongly regulated by FBS deprivation but only marginally by arginine deprivation, suggesting an important regulatory role of growth factors in CASTOR1 protein level. Treatment with AKT inhibitor MK2206 in 293T cells upregulated CASTOR1 protein but not mRNA level, and decreased mTORC1 activation, mimicking FBS deprivation (FIG. 1C, and FIG. 6K). Because mTORC1 could be responsive to other nutrients such as leucine present the medium, the effect of leucine deprivation on CASTOR1 protein was further examined. Similar to arginine deprivation, chronic leucine deprivation activated AKT, reduced CASTOR1 mRNA and protein levels, and mTORC1 activity (FIGS. 6k and 6L). Interestingly, a S6K1 inhibitor that decreased the pS6K but not p4EBP1 level, failed to activate AKT and reduce CASTOR1 protein level (FIG. 6L). Together these results suggest the involvement of a regulatory role of CASTOR1 in the AKT-mTORC1 loop.

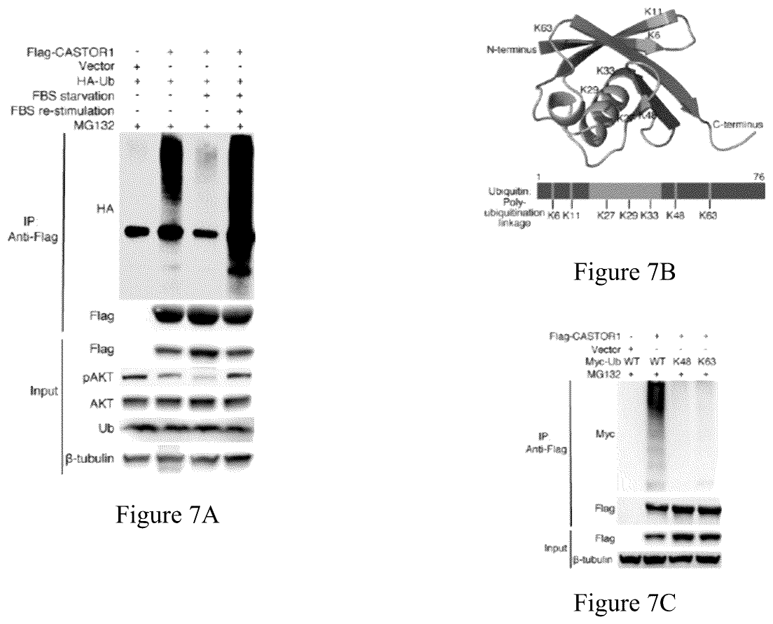

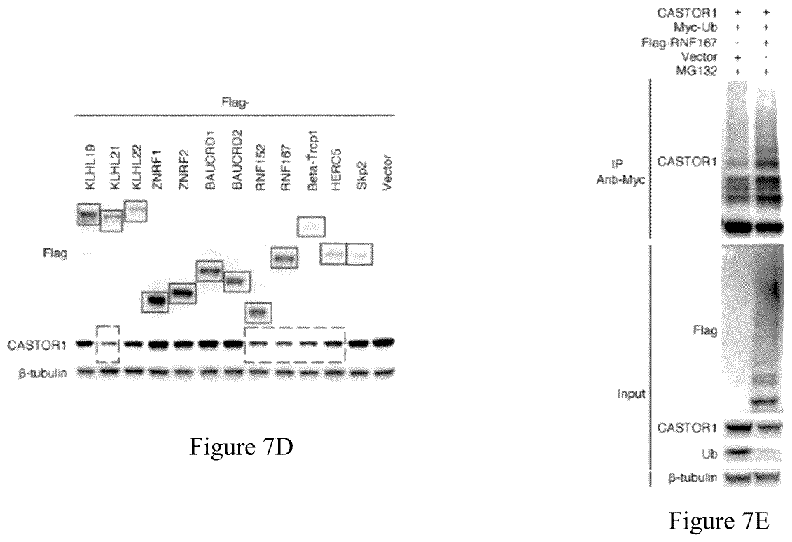

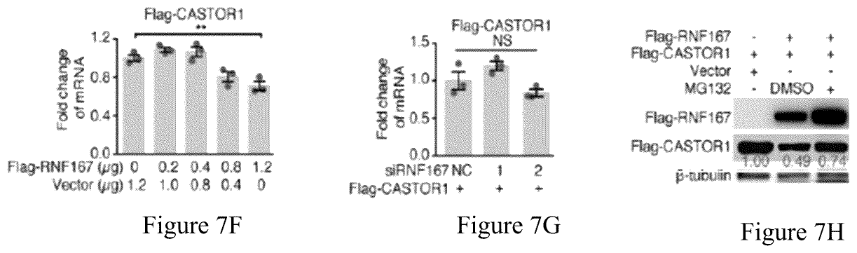

AKT1 phosphorylation of CASTOR1 promotes RNF167-mediated ubiquitination and degradation of CASTOR1. Since these results suggested that the CASTOR1 protein level was strongly regulated by FBS, potentially through AKT activation, the mechanism mediating CASTOR1 degradation was examined. Consistent with the observed CASTOR1 protein level, FBS deprivation reduced CASTOR1 ubiquitination, while arginine deprivation had no noticeable effect (FIG. 1D). FBS re-stimulation after deprivation reversed the effect, restoring CASTOR1 ubiquitination, which was correlated with the reduced CASTOR1 ubiquitination and protein level (FIG. 7A). Together, these results confirmed that arginine did not significantly affect CASTOR1 protein level but FBS targeted CASTOR1 for ubiquitination and proteasome-dependent degradation.

Covalent conjugation of ubiquitin is a key step in proteasome-mediated degradation of target proteins. CASTOR1 was only labeled by wild-type (WT) ubiquitin or K29 ubiquitin, a ubiquitin mutant containing only the K29 lysine but not by K48 and K63 ubiquitin (FIG. 1E, and FIGS. 7B and 7C). Mutation of K29 ubiquitin (K29R) abolished CASTOR1 ubiquitination (FIG. 1E). These results indicated that K29 ubiquitin was essential and sufficient to mediate CASTOR1 ubiquitination.

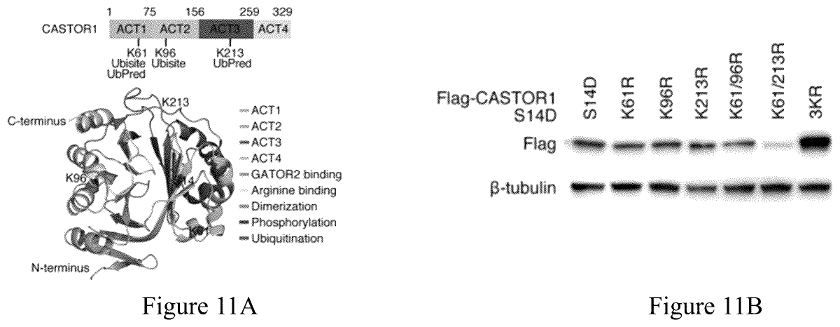

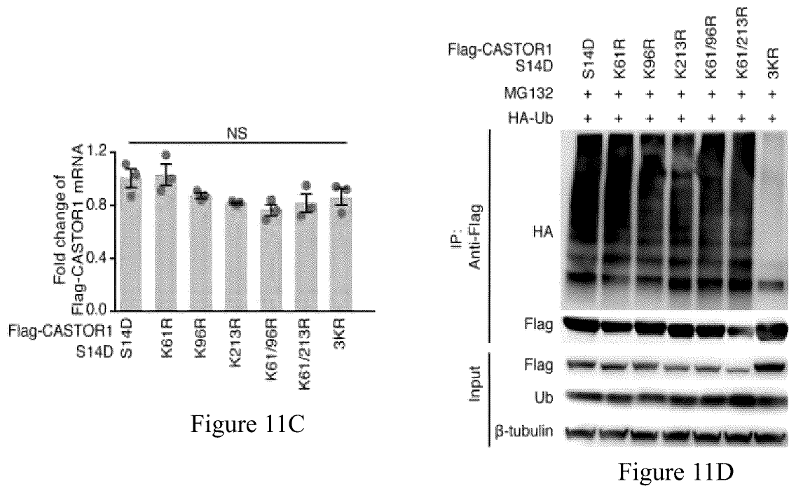

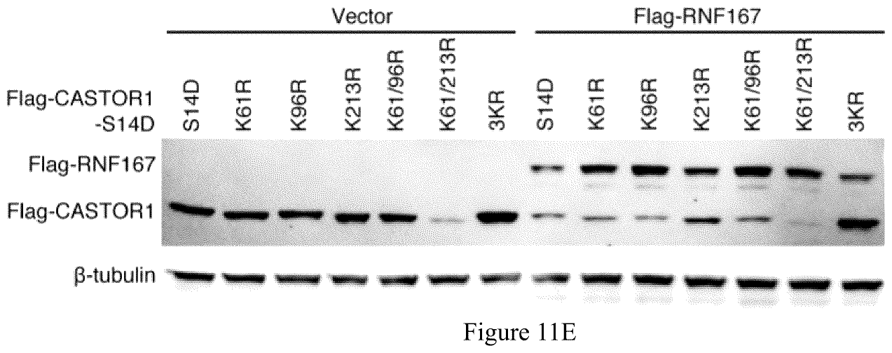

To identify the E3 ubiquitin ligase(s) that might regulate CASTOR1 polyubiquitination and degradation, a panel of E3 ubiquitin ligases implicated in mTORC1 regulation was screened. Although ectopic expression of numerous E3 ubiquitin ligases decreased CASTOR1 protein level (FIG. 7D), only RNF167 increased CASTOR1 ubiquitination (FIG. 1F, and FIG. 7E). Consistently, knockdown of RNF167 decreased CASTOR1 ubiquitination and increased CASTOR1 protein level (FIGS. 1G and 1H) while overexpression of RNF167 decreased CASTOR1 protein level in a dose-dependent manner (FIG. 11). Neither overexpression nor knockdown of RNF167 had a notable effect on the CASTOR1 mRNA level (FIGS. 7F and 7G). Additionally, treatment with MG132 partially rescued RNF167-mediated downregulation of CASTOR1 protein (FIG. 7H). These results support a model that RNF167 targets CASTOR1 for ubiquitination and proteasome-dependent degradation (FIG. 1J).

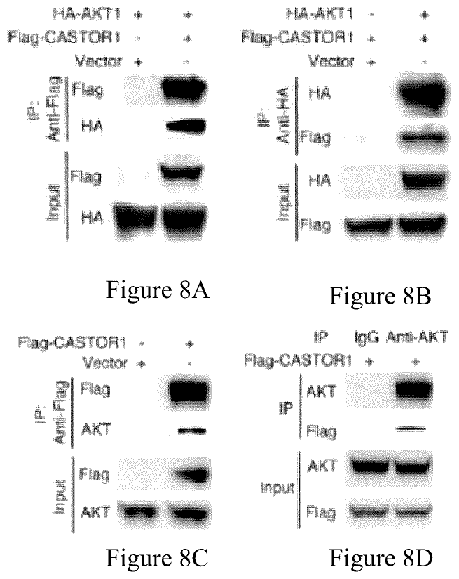

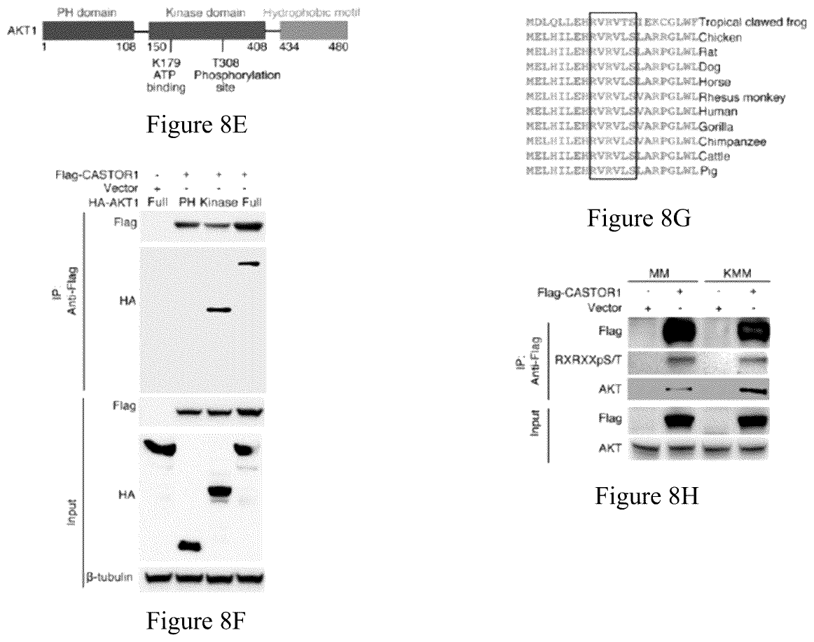

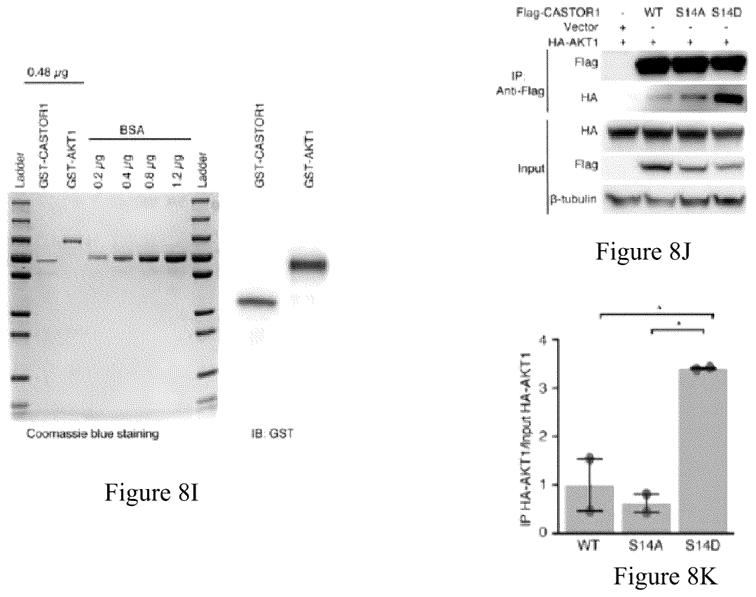

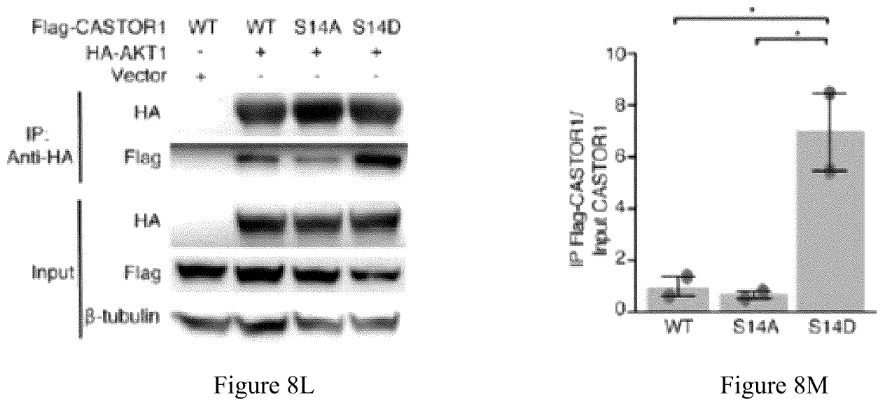

By providing growth factors, FBS activates numerous kinases, which could be the reason that it regulates the CASTOR1 level. Since the effect of AKT inhibitor MK2206 on the CASTOR1 protein level was the same as FBS starvation (FIG. 1C), kinase prediction algorithms were used to identify a consensus AKT1 phosphorylation site on CASTOR1 with a motif of R—V—R—V-L-S14 (SEQ ID NO: 26). Proteomic analysis indeed identified CASTOR1 phosphorylation at S14, suggesting AKT1 might directly phosphorylate CASTOR1. Indeed, CASTOR1 interacted with both ectopically expressed AKT1 and endogenous AKT, and preferentially bound to AKT1 kinase domain (FIG. 8A-8F). An antibody specific to the AKT phosphorylation consensus motif (R—X—R—X—X-pS/T) detected a strong signal in the WT HA- or Flag-CASTOR1 protein expressed in 293T cells, confirming that CASTOR1 was phosphorylated at the physiological condition (FIGS. 2A and 2B). Importantly, the level of CASTOR1 phosphorylation was positively correlated with AKT activation, which was increased following deprivation of arginine or leucine but decreased following deprivation of FBS or all amino acids (AA). See FIG. 2A. Furthermore, CASTOR1 protein level was negatively correlated with CASTOR1 phosphorylation at S14 (FIG. 2A)), suggesting that AKT mediated CASTOR1 phosphorylation at S14 to target its degradation. In agreement with these results, an alanine substitution at S14 (Flag-CASTOR1 S14A), which generated a phosphorylation dead mutant, and AKT inhibitor MK2206 significantly reduced the specific phosphorylation of the AKT motif (FIGS. 2B and 2C), hence confirming AKT-mediated phosphorylation of CASTOR1 at S14. The alignment of CASTOR1 protein sequences from humans with other vertebrates revealed that the CASTOR1 R—X—R—X—X-S14 motif was highly conserved (FIG. 8G). As expected, AKT interacted with and phosphorylated CASTOR1 at the AKT phosphorylation motif in rat metanephric mesenchymal precursor (MM) cells and KSHV-transformed MM (KMM) cells (FIG. 8H).

In vitro kinase assay was performed to confirm AKT direct phosphorylation of CASTOR1. Purified glutathione S-transferase (GST)-AKT1 efficiently phosphorylated purified GST-tagged CASTOR1 (GST-CASTOR1) recombinant protein only in the presence of ATP, which was abolished by AKT inhibitor MK2206 (FIG. 2D, and FIG. 8I). Interestingly, Flag-CASTOR1 S14D, a mimic of constitutively phosphorylated mutant, had a much higher affinity to AKT1 than Flag-CASTOR1 WT and Flag-CASTOR1 S14A (FIG. 8J-8M), suggesting possible CASTOR1 conformational changes following phosphorylation. A similar observation that the AKT3-Ago2 interaction was enhanced following AKT3 phosphorylation of Ago2 at S387 was previously reported. Collectively, these results demonstrated that AKT is directly bound to and phosphorylated CASTOR1.

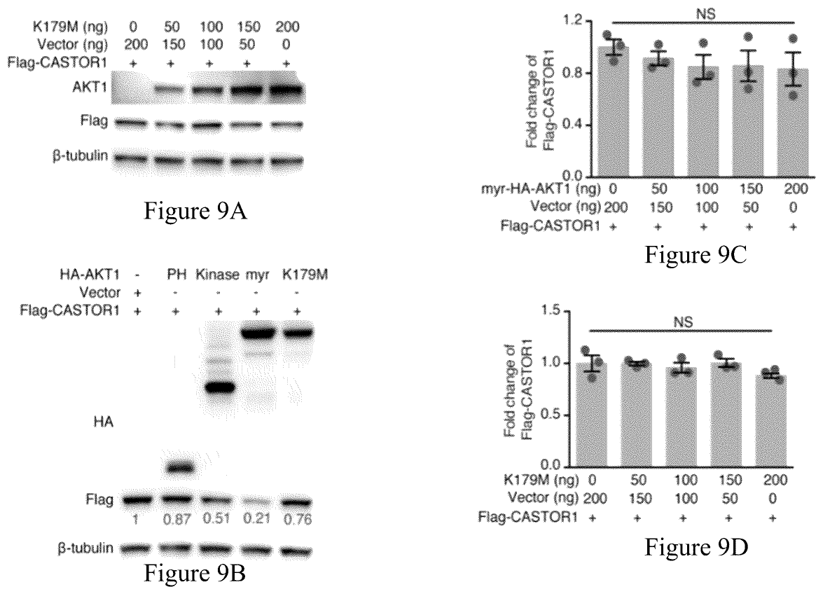

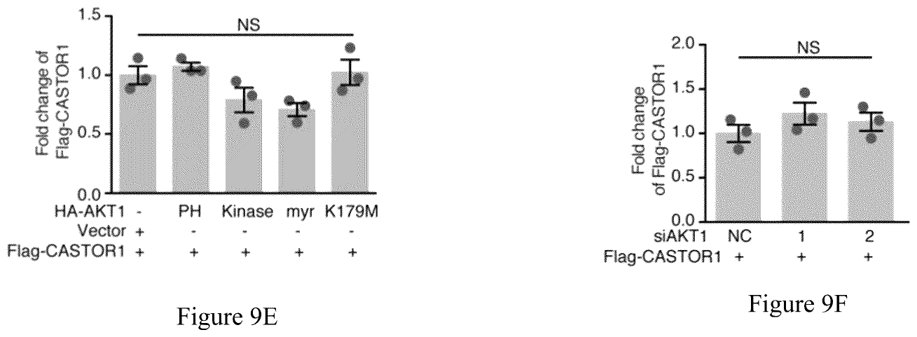

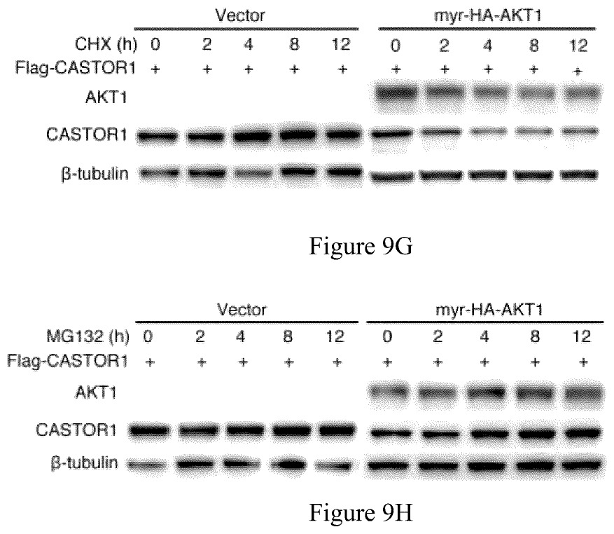

As phosphorylation is intimately linked to protein ubiquitination and degradation, the consequence of AKT1-mediated CASTOR1 phosphorylation was examined, and myristoylated constitutively active AKT1 (myr-HA-AKT1) decreased CASTOR1 protein level in a dose-dependent manner (FIG. 2). Neither the kinase-dead AKT1 mutant (K179M) nor the AKT1 PH domain had any effects, while overexpression of the AKT1 kinase domain alone was sufficient to reduce the CASTOR1 protein level albeit to a lesser degree than the WT AKT1 (FIG. 9A-9B). Hence, AKT-mediated CASTOR1 downregulation required its kinase activity. Neither the WT AKT1, AKT1 PH, and kinase domains nor the kinase-dead mutant affected the CASTOR1 mRNA level (FIG. 9C-9E). Consistently, AKT1 silencing was sufficient to inhibit pan AKT activity, and increased CASTOR1 protein level (FIG. 2F) but had no effect on the CASTOR1 mRNA expression (FIG. 9F).

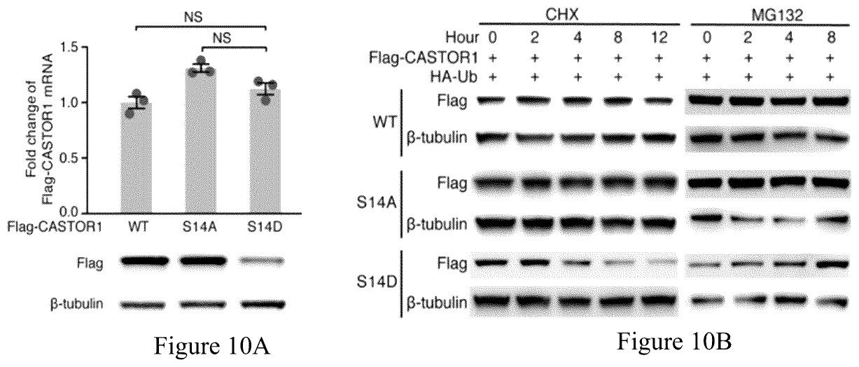

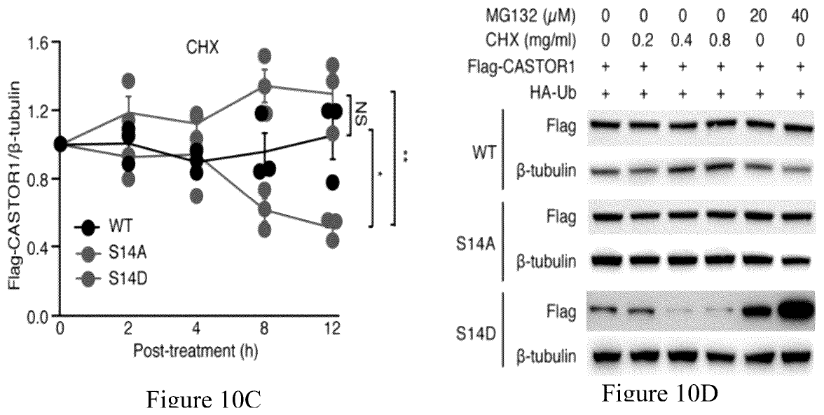

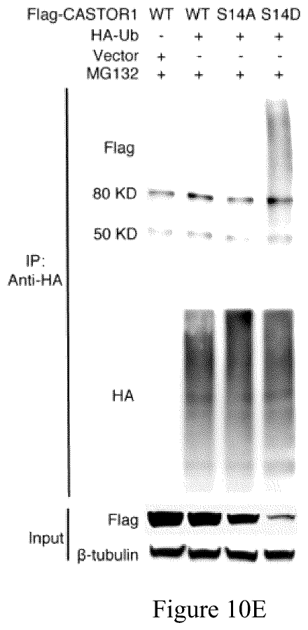

To test whether AKT1 regulates CASTOR1 stability, cells were co-transfected with both Flag-CASTOR1 WT and myr-HA-AKT1, then treated them with de novo protein synthesis inhibitor cycloheximide (CHX), and observed faster degradation of CASTOR1 protein in cells expressing myr-HA-AKT1 than the vector control (FIG. 9G). Treatment with proteasome inhibitor MG132 increased the accumulation of CASTOR1 protein in cells expressing myr-HA-AKT1 but only had a marginal effect on cells expressing the vector control (FIG. 9H). Furthermore, overexpression of myr-HA-AKT1 but not AKT1 mutant (K179M) enhanced whereas knockdown of AKT1 reduced CASTOR1 ubiquitination (FIGS. 2G and 2H). Together, these results confirmed that AKT1 targeted CASTOR1 for ubiquitination and proteasome-dependent degradation. 293T cells was constructed stably expressing Flag-CASTOR1 WT, S14A or S14D, and observed that cells expressing Flag-CASTOR1 S14D had lower protein level than those expressing CASTOR1 WT and S14A despite there was no significant change at the mRNA level (FIG. 10A). Indeed, treatment with CHX reduced while treatment with MG132 increased S14D protein level but had minimal effects on WT and S14A (FIG. 10B-10D). Accordingly, the level of ubiquitination was significantly increased for S14D protein compared to those of WT and S14A proteins (FIG. 2I and FIG. 10E). These results demonstrated that AKT1 phosphorylation of CASTOR1 at S14 resulted in its ubiquitination and degradation.

To clarify the link between AKT1-mediated phosphorylation and RNF167-mediated ubiquitination of CASTOR1, the effect of CASTOR1 phosphorylation on CASTOR1-RNF167 interaction was examined. CASTOR1 S14D had a much stronger affinity to RNF167 and a higher level of ubiquitination than WT or S14A had (FIGS. 2I-2K, and FIG. 10E), indicating that AKT-mediated phosphorylation promoted CASTOR1 degradation by specifically enhancing the CASTOR1-RNF167 interaction. Collectively, these results support a model that AKT1 phosphorylation of CASTOR1 at S14 enhances RNF167-targeting ubiquitination and degradation of CASTOR1 protein.

Examination of CASTOR1 with the Ubisite and UbPreb program identified numerous lysine residues as putative ubiquitination sites including K61, K96, and K213 (FIG. 11A). Whereas mutation of one or two of these sites to arginine in the CASTOR1 S14D failed to stabilize the protein, mutation of all three sites to arginine (3KR) significantly blocked CASTOR1 ubiquitination and degradation (FIG. 111B-11D). Importantly, while all single and double lysine mutants of CASTOR1 S14D remained sensitive to RNF167-mediated downregulation, the 3KR mutant was resistant (FIG. 11E), indicating that RNF167 catalyzed CASTOR1 ubiquitination at multiple lysines.

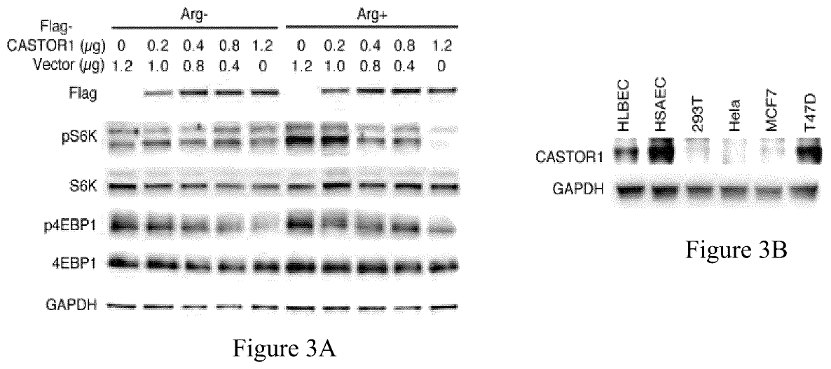

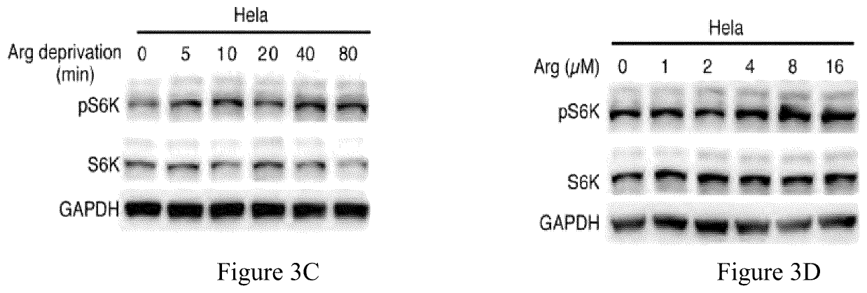

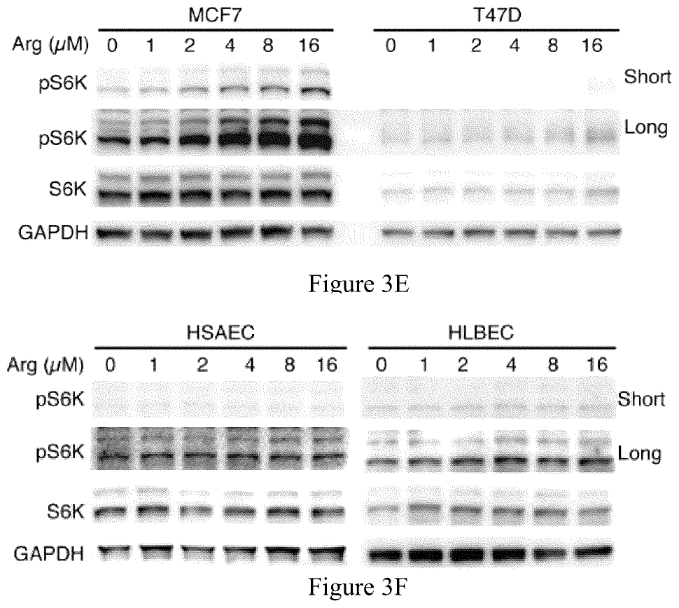

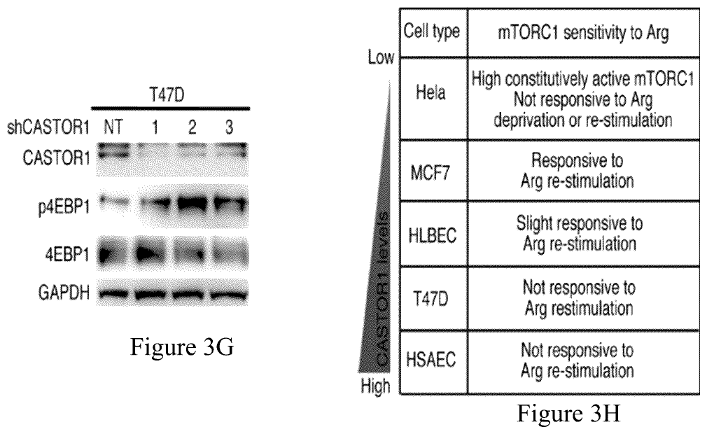

High CASTOR1 protein level overrides arginine activation of mTORC1: Next, the downstream effects of AKT1-mediated phosphorylation and RNF167-targeting degradation of CASTOR1 protein were assessed. Consistent with the previous report, a high level of CASTOR1 protein rendered cells insensitive to arginine-mediated activation of mTORC1 in 293T cells (FIG. 3A). To determine whether CASTOR1 regulates mTORC1 activation in physiological conditions, CASTOR1 protein levels in different types of cells and their sensitivities to arginine were examined (FIG. 3B). Hela cells, which had almost no detectable CASTOR1 protein expression, were resistant to mTORC1 inactivation by arginine deprivation (80 min) as well as mTORC1 activation by 10 min arginine re-stimulation following 50 min arginine deprivation (i.e., arginine-mediated mTORC1 activation, FIG. 3B-3D). These results indicated that mTORC1 was constitutively activated when CASTOR1 protein expression was completely silenced, and that these cells were no longer responsive to arginine. MCF7 cells, which had a low CASTOR1 protein level, were responsive to arginine-mediated mTORC1 activation (FIG. 3B, 3E). In contrast, cells with high endogenous CASTOR1 protein levels including human lobar bronchial epithelial cells (HLBEC), human small airway epithelial cells (HSAEC) and T47D were not responsive to arginine-mediated mTORC1 activation (FIG. 3B, 3E, 3F), suggesting CASTOR1's strong suppressive role in mTORC1 activity in these cells. Under this condition, no CASTOR1 protein level change was observed in these cells (results not shown). In agreement with these results, silencing of CASTOR1 in T47D cells, which had a high endogenous CASTOR1 protein level, was sufficient to strongly activate mTORC1, further supporting CASTOR1's direct regulatory role in mTORC1 activity (FIG. 3B, 3G). Together, these results supported the notion that a high CASTOR1 protein level overrode arginine activation of mTORC1 at physiological conditions, and the mTORC1 activity was tightly regulated by CASTOR1 instead of the arginine status when CASTOR1 was expressed at a high level (FIG. 3H).

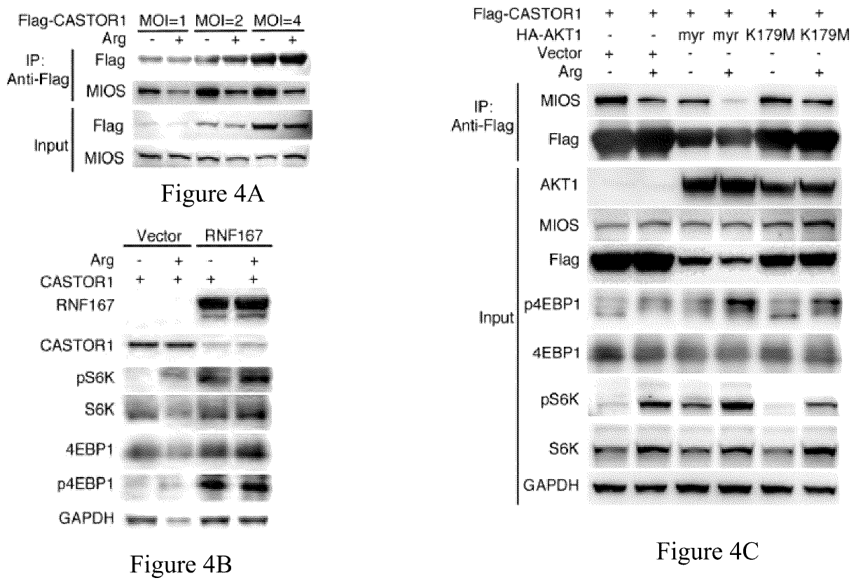

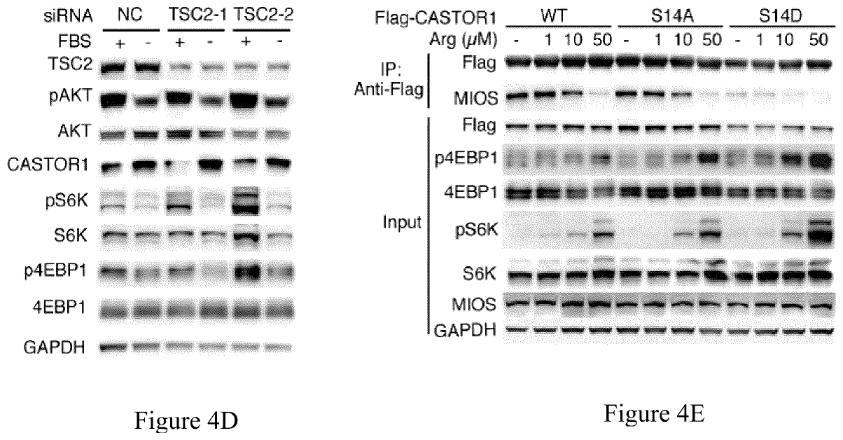

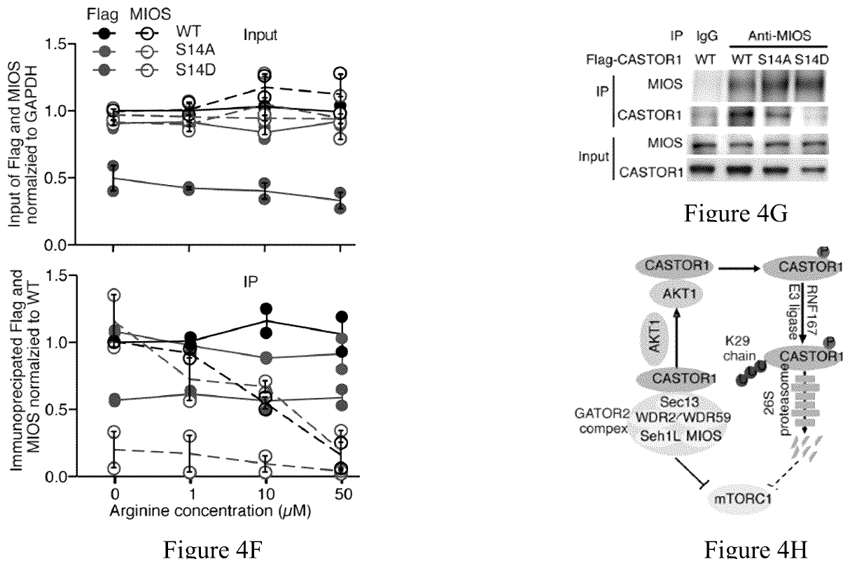

RNF167-mediated ubiquitination and AKT-mediated phosphorylation of CASTOR1 release mTORC1 inactivation: Mechanistically, binding of CASTOR1 to MIOS, the core component of GATOR2 complex, was positively correlated with the CASTOR1 protein level, further demonstrating that mTORC1 activation was regulated by CASTOR1 protein level in addition to arginine (FIG. 4A). As expected, ectopic expression of RNF167 degraded CASTOR1 and activated mTORC1 regardless the presence or absence of arginine (FIG. 4B). In fact, cells with overexpression of RNF167 became insensitive to arginine-mediated mTORC1 activation (FIG. 4B), affirming the essential role of RNF167 and regulation of CASTOR1 protein level in the control of mTORC1 activation. As expected, myr-HA-AKT1 but not its kinase-dead mutant K179M decreased CASTOR1 protein level and hence its binding to MIOS, resulting in increased mTORC1 activation (FIG. 4C). The consensus mechanism of AKT-mediated activation of mTORC1 is by suppressing TSC2. However, these results suggested that AKT might also activate mTORC1 by targeting CASTOR1 for degradation. To dissect AKT's independent effects on CASTOR1 and TSC2 in regulating mTORC1, knockdown of TSC2 was performed, and CASTOR1 expression and mTORC1 activity in the presence or absence of FBS were examined. As expected, FBS deprivation reduced AKT activation and increased the CASTOR1 protein level leading to mTORC1 inactivation in controlled cells (FIG. 4D). Silencing of TSC2 had no effect on AKT activation and CASTOR1 protein level but was sufficient to activate mTORC1. However, mTORC1 was still inactivated by the increased CASTOR1 protein level following FBS deprivation in the TSC2 silencing cells (FIG. 4D). These results indicated that AKT activated mTORC1 through two independent pathways, by reducing CASTOR1 protein level and by suppressing TSC2.

Since CASTOR1 S14D was constitutively phosphorylated and hence was prone to degradation whereas CASTOR1 S14A was non-phosphorylatable and resistant to degradation, these constructs were used to assess the effect on mTORC1 activation. Consistently, the protein level was lower, which led to a lower pull down yield in co-immunoprecipitation, for S14D than WT and S14A (FIG. 4E, 4F). Furthermore, S14D binding to MIOS was significantly weaker than that of WT or S14A even after taking into consideration of its lower protein level and lower pull down efficiency in co-immunoprecipitation (FIG. 4E-4G). Hence, a lower protein level and a lower affinity to MIOS might lead to a more robust mTORC1 activation for S14D than WT and S14A (FIG. 4E, 4G). These differences persisted even with arginine concentration reaching 50 μM indicating that the combined effects of AKT1 phosphorylation and RNF167-targeting degradation had a stronger role than arginine inhibition of CASTOR1 in regulating mTORC1 activation, particularly at a condition with a low concentration of arginine, which is common in tumor microenvironment (FIG. 4H).

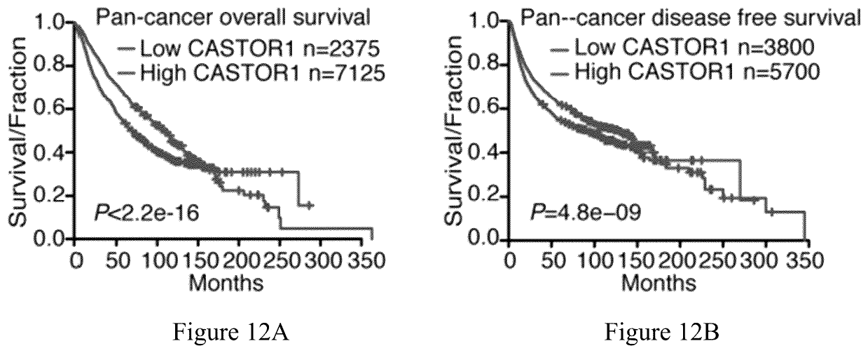

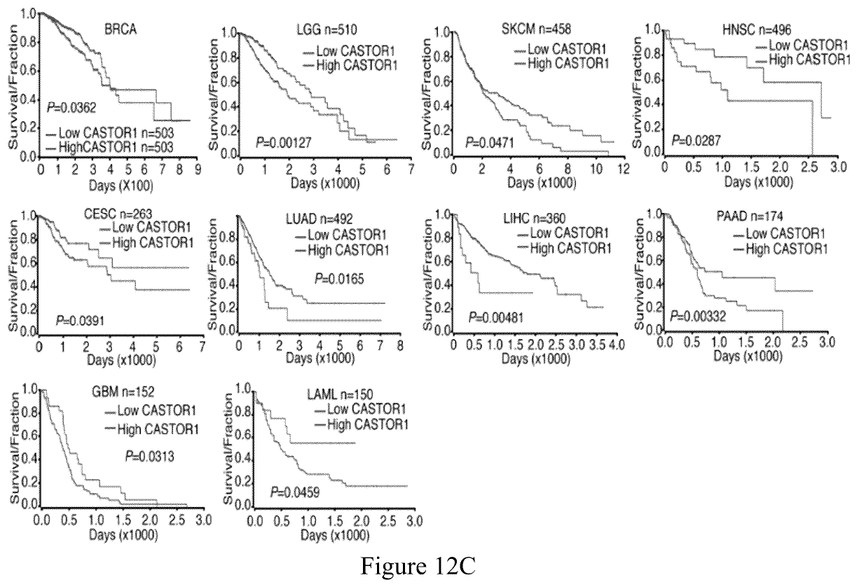

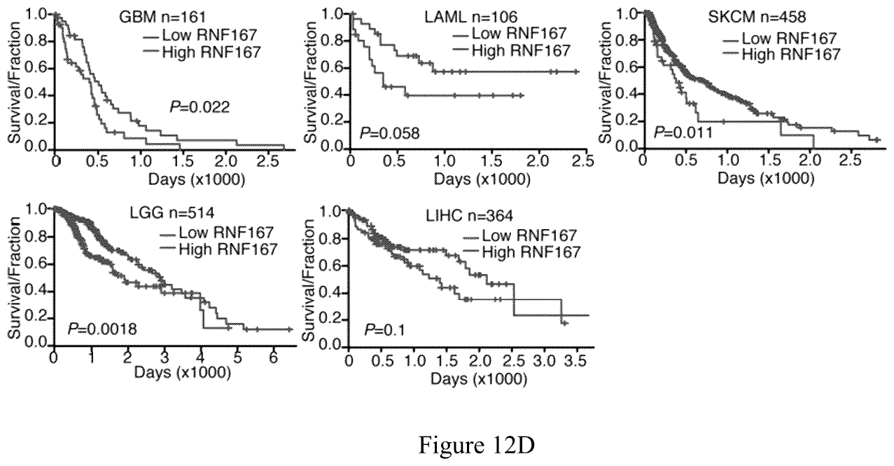

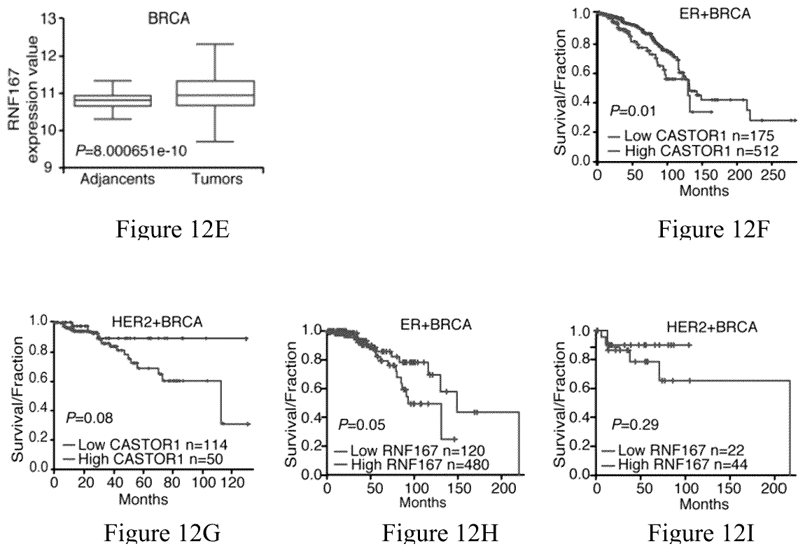

RNF167-mediated ubiquitination and AKT1-mediated phosphorylation of CASTOR1 promote breast cancer progression: the prognostic value of CASTOR1 mRNA expression in cancer was predicted using the TCGA database. Consistent with CASTOR1's inhibitory function on mTORC1 and tumor suppressive role, a lower CASTOR1 expression level was correlated with overall poor survival in pan-cancer analyses (FIG. 12A, 12B). At least 10 types of cancer showed a strong negative correlation including breast invasive carcinoma (BRCA), brain lower grade glioma (LGG), skin cutaneous melanoma (SKCM), head and neck squamous cell carcinoma (HNSC), cervical squamous cell carcinoma and endocervical adenocarcinoma (CESC), lung adenocarcinoma (LUAD), liver hepatocellular carcinoma (LIHC), pancreatic adenocarcinoma (PAAD), glioblastoma multiforme (GBM) and acute myeloid leukemia (LAML) (FIG. 12C), of these, high RNF167 expression predicted a poor prognosis in GBM, LAML, SKCM, LGG and LIHC (FIG. 12D).

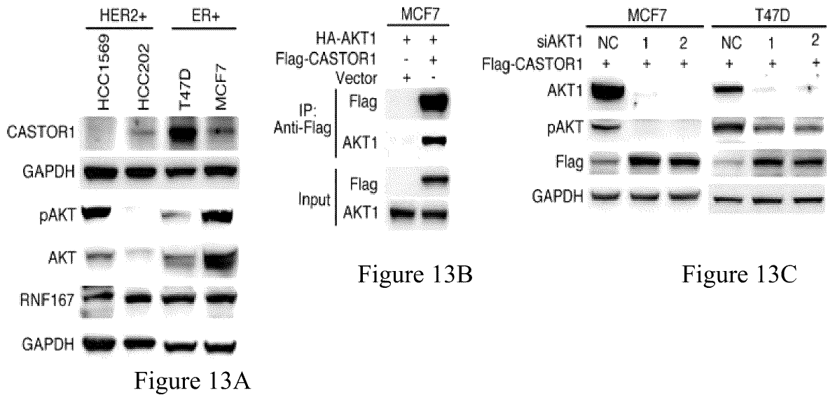

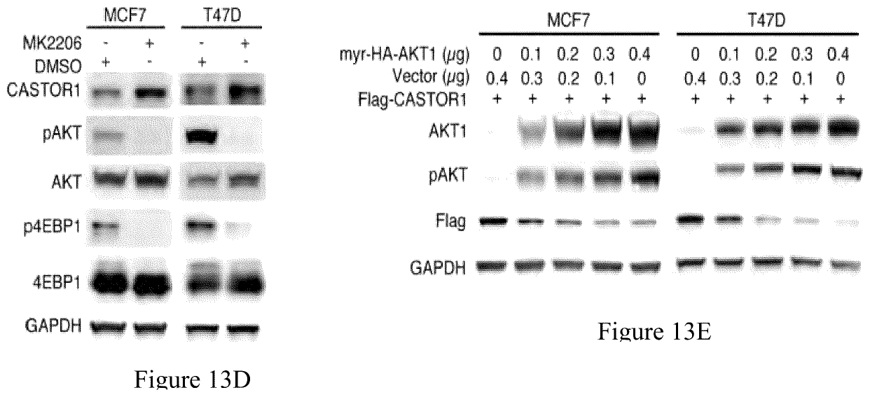

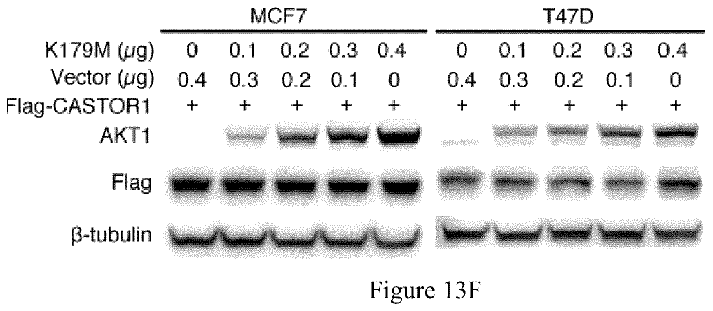

Breast cancer represents 12% of cancer diagnosed and is a major life threat for women in the United States. A high RNF167 expression level was found in breast tumors compared to the adjacent normal tissues (FIG. 12E). Furthermore, a lower CASTOR1 expression level (FIG. 12F, 12G) and a higher RNF167 expression level (FIG. 12H, 12I) were correlated with poor survival in ER+ and HER2+ breast cancer, respectively. In two pairs of ER+ and HER2+ breast cancer cell lines, an inverse correlation of activated AKT level with CASTOR1 protein level was identified (FIG. 13A). AKT interacted with CASTOR1 in MCF cells (FIG. 13B). Silencing of AKT1 and AKT inhibitor MK2206 enhanced exogenous and endogenous CASTOR1 protein levels in these cells, respectively (FIG. 13C, 13D). Consistently, overexpression of myr-HA-AKT1 but not the AKT kinase dead mutant K179M in MCF7 and T47D cells resulted in a dose-dependent reduction in CASTOR1 protein level (FIG. 13E, 13F).

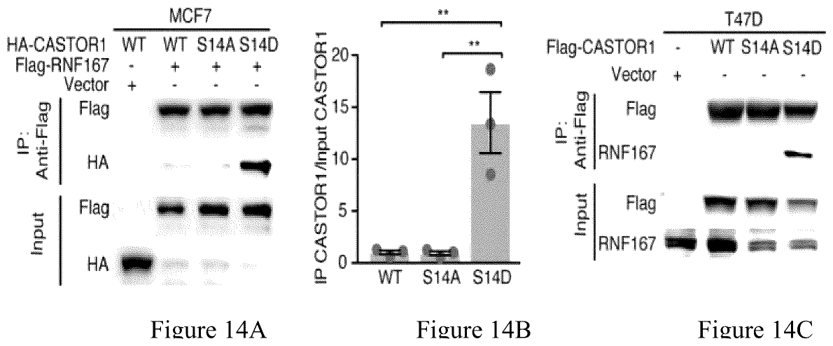

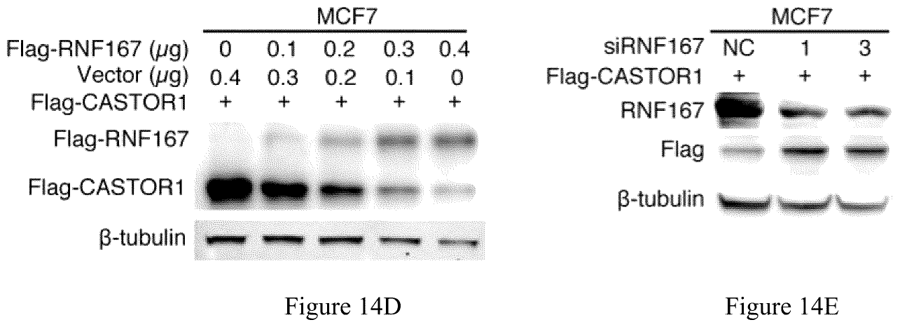

Consistent with 293T cells, the affinity to exogenous and endogenous RNF167 was stronger for CASTOR1 S14D than WT and S14A in ER+ MCF7 and T47D cells, respectively (FIG. 14A-14C). Likewise, RNF167 overexpression decreased whereas RNF167 knockdown increased CASTOR1 protein level in MCF7 cells (FIG. 14D, 14E). Together these results indicated that similar to 293T cells, the CASTOR1 protein level was also regulated by AKT and RNF167 in breast cancer cells.

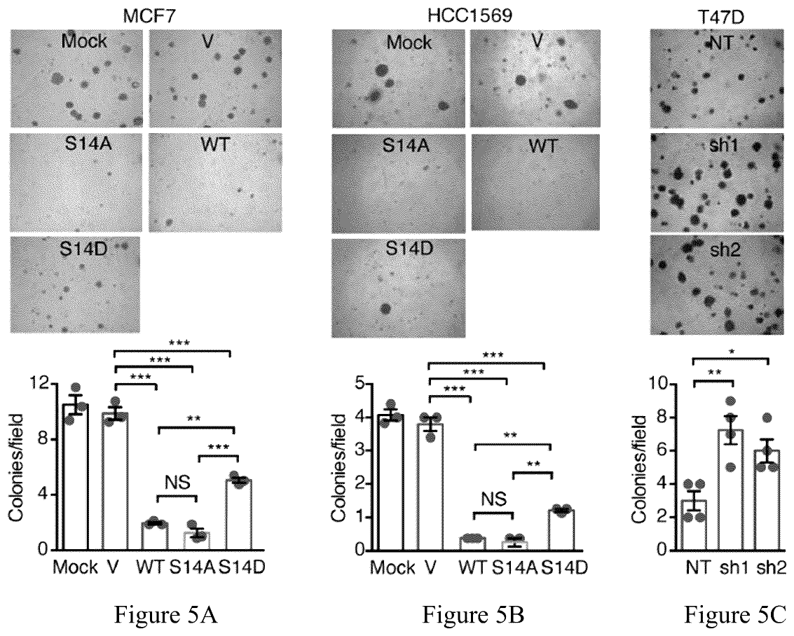

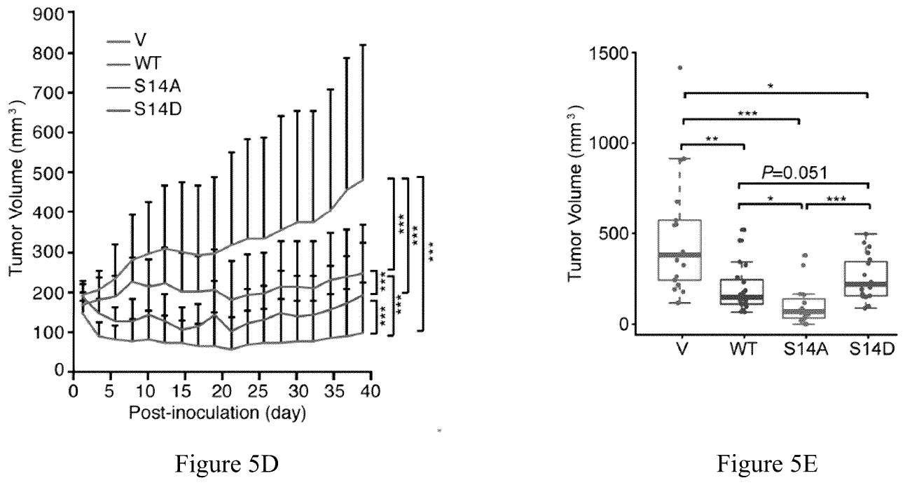

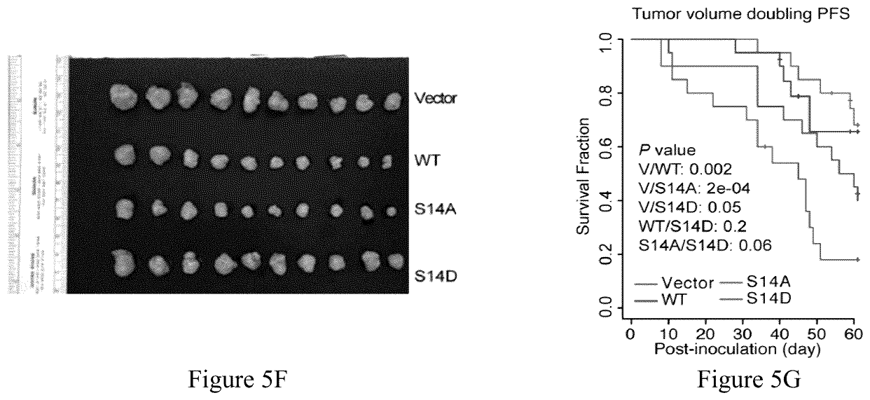

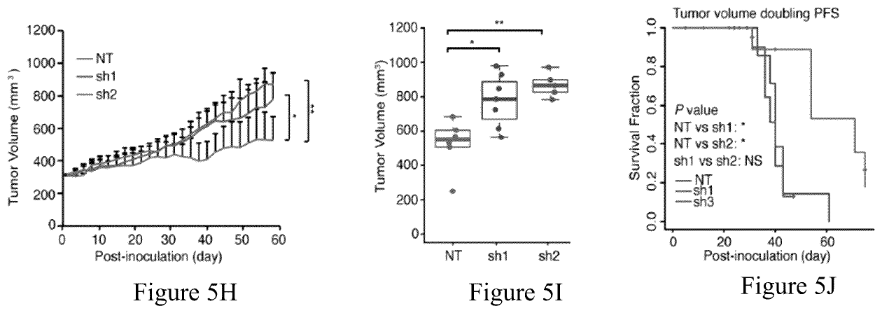

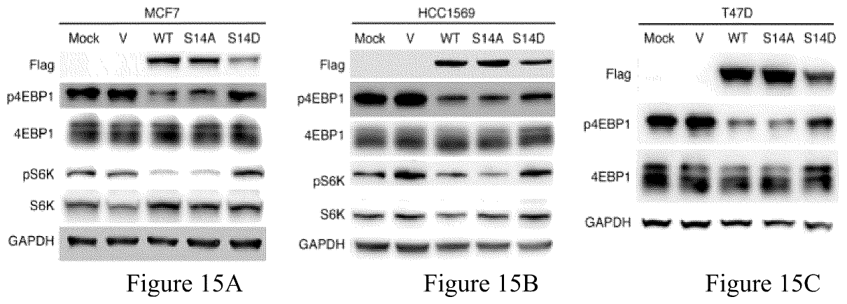

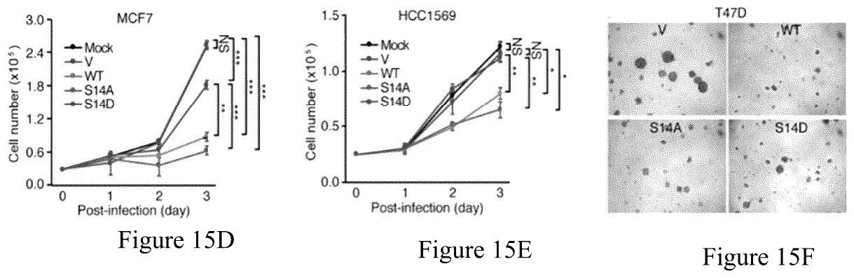

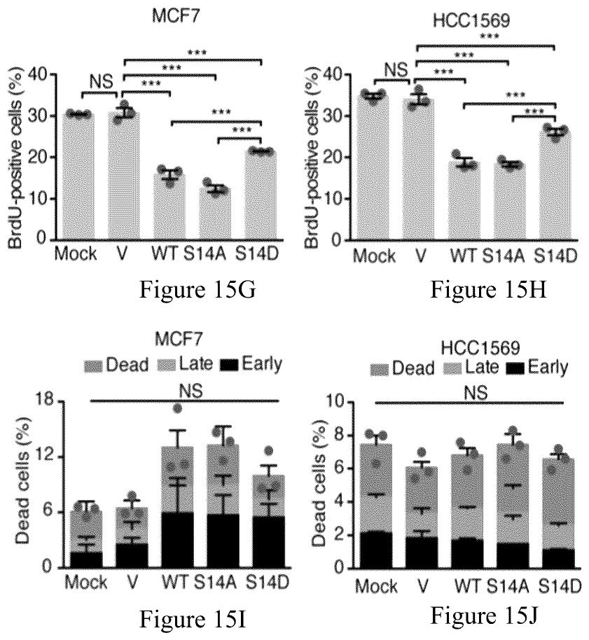

To examine the importance of AKT1-mediated phosphorylation and RNF167-mediated degradation of CASTOR1 in breast cancer cells, Flag-CASTOR1 WT, S14A, and S14D were overexpressed in HCC1569, MCF7, and T47D cells. CASTOR1 S14D had much lower expression level than WT and S14A had in all three cell lines examined (FIG. 15A-15C), indicating that S14D also had a faster turnover in breast cancer cells. Importantly, ectopic expression of both CASTOR1 WT and S14A significantly inhibited mTORC1, whereas CASTOR1 S14D showed a much less inhibitory effect, confirming the fine-tuning of mTORC1 signaling pathway through CASTOR1 phosphorylation and degradation in breast cancer cells (FIG. 15A-15C). Consistent with the mTORC1 activity, the proliferation and colony formation in softagar of breast cancer cells was significantly decreased by CASTOR1 WT and S14A, whereas CASTOR1 S14D had a less effect (FIG. 5A, 5B, and FIG. 15D-15F). In T47D cells, which had a high endogenous level of CASTOR1 protein, silencing of CASTOR1 activated mTORC1 and significantly increased the colony formation efficiencies in softagar (FIG. 3G, and FIG. 5C). Moreover, overexpression of CASTOR1 WT and S14A had a stronger effect than S14D had in inhibiting cell cycle progression in MCF7 and HCC1569 cells (FIG. 15G-15H). None of the CASTOR1 constructs had any significant effect on apoptosis (FIG. 15I-15J), which recapitulated the characteristic phenotype of mTORC1 inhibition.

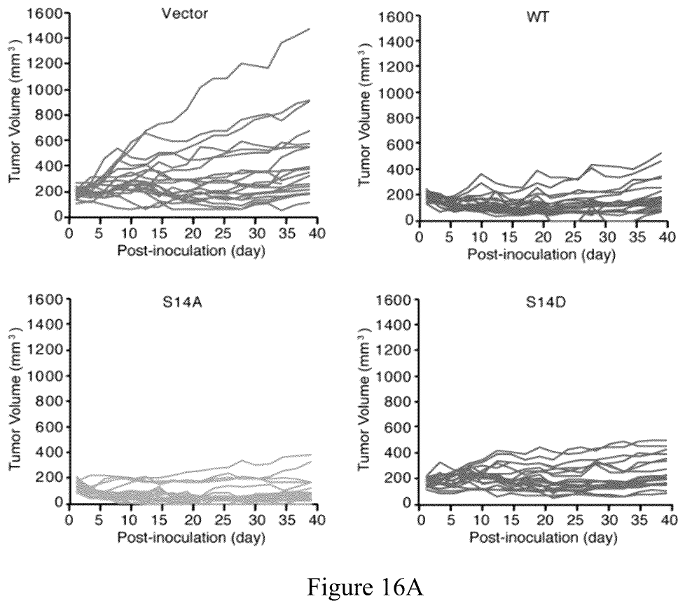

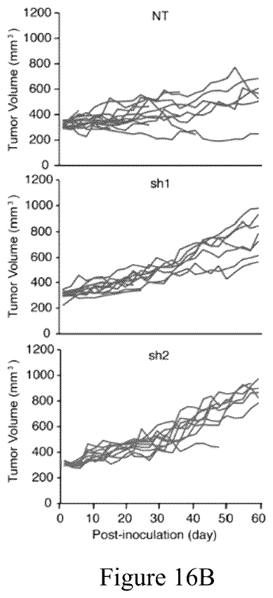

MCF7 cells that were transduced with a vector control, Flag-tagged CASTOR1 WT, S14A or S14D are subcutaneously engrafted into both flanks of nude mice. Ectopic expression of CASTOR1 WT and S14A significantly inhibited tumor growth in vivo, whereas S14D had a relatively less effect (FIGS. 5D-5F, and FIG. 16A). Additionally, mice injected with cells expressing CASTOR1 WT and S14A had higher survival rates than those of expressing vector control and S14D (FIG. 5G). Consistently, silencing of CASTOR1 in T47D cells promoted tumor growth in vivo and shortened the overall survival compared to a scrambled control group (FIGS. 5H-5J, and FIG. 16B). Taken together, these results revealed that AKT-mediated phosphorylation and RNF167-dependent ubiquitination led to a decreased CASTOR1 protein level in breast cancer cells, resulting in enhanced mTORC1 activation, cell proliferation, and tumorigenesis.

### DISCUSSION

A general mechanism of AKT-mediated phosphorylation at S14 and RING-type E3 ligase RNF167-mediated ubiquitination at multiple lysine residues of CASTOR1 leading to its proteasome-dependent degradation and consequently mTORC1 activation is assessed. The AKT phosphorylation site in CASTOR1 is present in other vertebrate species analyzed, indicating its conserved function. Mutation of this site into a constitutively phosphorylated mutant (S14D) increases its interaction with AKT, suggesting a possible conformation change and a feed-forward negative AKT regulatory mechanism of the CASTOR1 protein. The CASTOR1 lysines, i.e., K61, K96 and K213 are marked by K29-linked polyubiquitination. Intriguingly, the constitutively phosphorylated S14D mutant has a significantly higher affinity to RNF167, explaining its faster ubiquitination and degradation, and a significantly lower affinity to MIOS. In addition, CASTOR1 phosphorylation at S14 significantly inhibits CASTOR1 dimerization, which is important for binding to the GATOR2 complex (FIG. 33). Hence, AKT-mediated CASTOR1 phosphorylation results in reduced CASTOR1 protein level and inhibition of the GATOR2 complex, both contributing to mTORC1 activation. This mechanism remains functional even after TSC2 knockdown indicating the presence of a TSC2-independent but CASTOR-dependent pathway of AKT-mediated mTORC1 activation. Importantly, by manipulating extracellular nutrients such as FBS and arginine in several types of cells, this mechanism of AKT-mediated CASTOR1 degradation and mTORC1 activation is functional in physiological conditions.

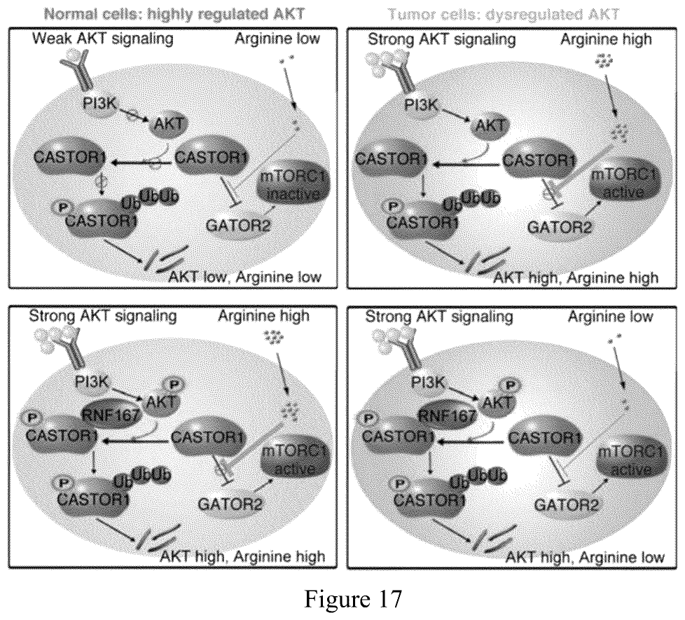

mTORC1 activation is tightly regulated occurring in a cascade fashion initiated by amino acids-mediated mTORC1 translocation to lysosomes followed by AKT-induced Rheb phosphorylation of mTOR. So far, several amino acid sensors including Sestrin2, SLC39A9, TM4SF5, and SAMTOR are known to modulate mTORC1 activity in response to amino acid status. CASTOR1 is a newly discovered arginine sensor, which interplays with arginine to modulate mTORC1 signaling pathway. Hence, these findings reveal a cross talk between two previously independent signaling pathways, i.e., the growth factor-dependent AKT and arginine-regulated CASTOR1 signaling pathways, which fine-tunes mTORC1 activation. This regulatory mechanism is likely essential for controlling the homeostasis and proliferation of normal cells. In normal cells that are quiescent or at a low proliferating rate, AKT is inactivated, leading to upregulated CASTOR1, mTORC1 inactivation, and a decreased uptake of nutrients including arginine, which would have a minimal effect on CASTOR1's function and mTORC1 activation (FIG. 17). In hyperproliferating normal cells such as stimulated immune cells, a higher level of AKT activation would lead to a lower level of CASTOR1, an increased level of mTORC1 activation, and a higher level of uptake of nutrients including arginine, which would also inhibit CASTOR1 function, resulting in maximal mTORC1 activation (FIG. 17).

The mTORC1 pathway is often dysregulated in cancer, which is critical for the progression of cancer. While CASTOR1's mTORC1 inhibitory function is negated by arginine, a high level of CASTOR1 protein evades the effect of arginine and prevents arginine-mediated mTORC1 activation (FIG. 3). Furthermore, cancer cells often survive in an environment with low nutrients including a low level of arginine. Hence, it is expected that cancer cells would have evolved specific mechanisms to counter CASTOR1's inhibitory effect on mTORC1 in nutrients-deficient tumor microenvironment. In KSHV-transformed cells, KSHV-encoded miRNAs downregulate CASTOR1 to activate mTORC1. In other types of cancer, the AKT pathway is persistently activated as a result of mutation of AKT itself or its upstream pathways of growth factors³, which would phosphorylate CASTOR1 leading to its ubiquitination and degradation, and activation of mTORC1 regardless of the presence of high or low level of arginine (FIG. 17). Thus, cancer cells at least partially utilize constitutively active AKT to inhibit CASTOR1's function leading to constitutive mTORC1 activation.

While no consistent association of CASTOR1 mutation with any types of cancer has been identified so far, a lower mRNA expression level of CASTOR1 predicts a poor prognosis in 10 types of cancer (FIG. 12C). Importantly, a higher mRNA expression level of RNF167 predicts a poor prognosis in 6 of these 10 types of cancer (FIG. 12D). The fact that a low mRNA expression level of CASTOR1 and a high mRNA level of RNF167 predict a poor prognosis of these cancer types suggest the existence of an additional mechanism(s) regulating their mRNA expression. Pharmacological intervention of RNF167 leading to CASTOR1 activation could be considered as a potential therapeutic approach for these cancer types.

CASTOR1 is tumor suppressive in KSHV-induced cellular transformation and lung adenocarcinoma^{9,10}. In breast cancer cell lines, the protein level of CASTOR1 appears to be inversely correlated with the level of AKT activation (FIG. 13A). Overexpression of CASTOR1 decreases cell proliferation and colony formation in softagar of breast cancer cells while genetic silencing of CASTOR1 has the opposite effect (FIGS. 5A-5C, and FIG. 15D-15F). In a mouse tumor model, overexpression of WT CASTOR1 inhibits tumor growth and extends animal survival rate (FIG. 5D-5G). While the constitutively phosphorylated mutant S14D has an inhibitory reduced effect, the dead phosphorylated mutant inhibits tumor growth even more effective than the WT CASTOR1 (FIG. 5D-5G), possibly due to its dominant negative effect. Hence, these results have demonstrated a tumor suppressive function of CASTOR1 in breast cancer cells, which is negated by AKT-mediated phosphorylation. Whether CASTOR1 protein has a tumor suppressive function in other types of cancer remains to be investigated.

In addition to extracellular arginine deficiency commonly observed in the tumor microenvironment, the rate-limiting enzyme ASS1 responsible for intracellular de novo arginine synthesis is also frequently silenced in most cancer types. These cancer cells are arginine auxotrophic, which are the basis for clinical trials with pegylated arginine deiminase (ADI-PEG20) and human recombinant arginase. These regimens are expected to deprive cancer cells of arginine, leading to CASTOR1 activation, mTORC1 suppression and tumor regression. While tumors initially respond to ADI-PEG20, ASS1-deficient tumors eventually become resistant to these treatments at least in part by activating the PI3K/AKT pathway. It can be speculated that AKT activation would result in CASTOR1 degradation and mTORC1 activation, contributing to the resistance to the therapies. Hence, AKT-mediated degradation of CASTOR1 could be an important mechanism of resistance to cancer therapies designed to deplete cancer cells of arginine. In this context, combining arginine deprivation and AKT inhibition could be an attractive approach to overcome resistance to these cancer therapies.

### Example 2: Tumor Suppressive Function of CASTOR1

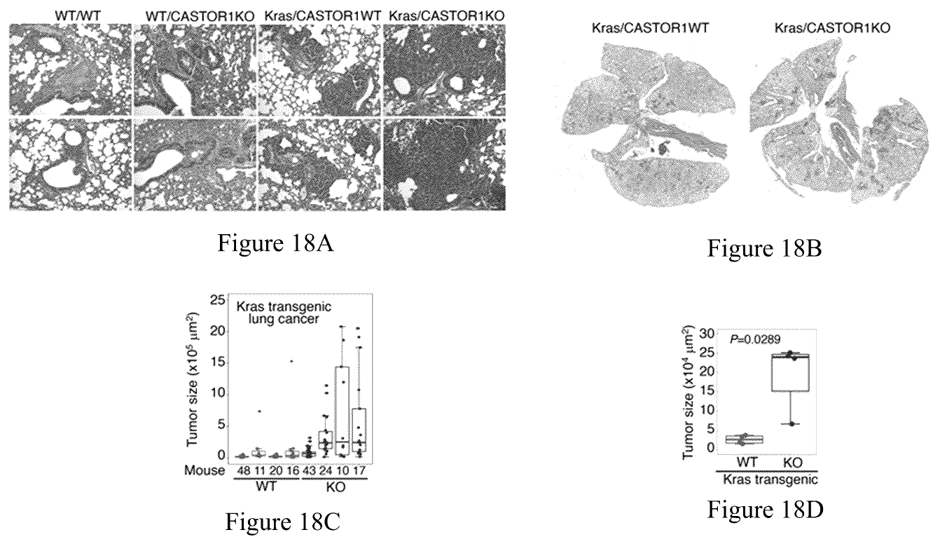

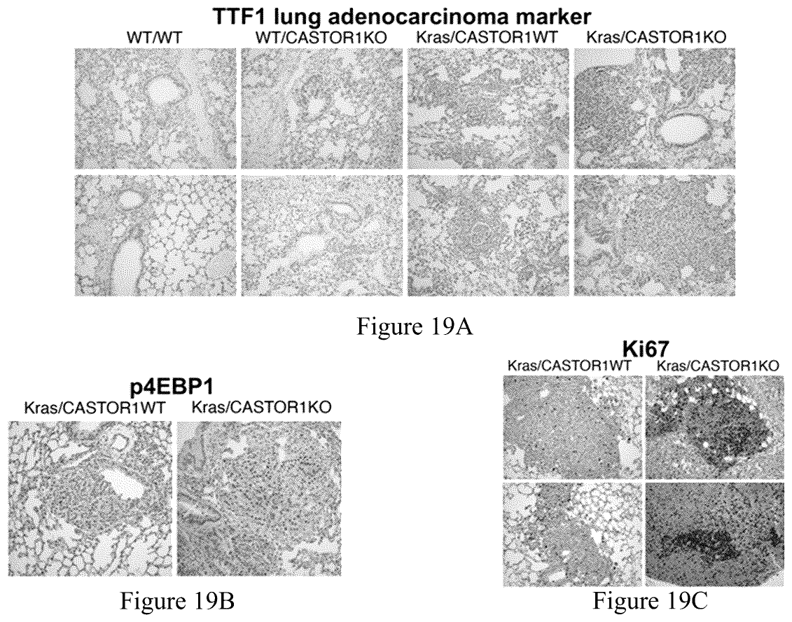

CASTOR1 is tumor suppressive in a Kras-driven mouse lung cancer model: To demonstrate the tumor-suppressive function of CASTOR1 in a genetic mouse model, a conditional Kras transgenic mouse was crossed with a CASTOR1 full-body knockout mouse. Intranasal inoculation of the mice with CRE-expressing adenoviruses of the mice induced the expression of Kras leading to the development of lung tumors. Kras induced more tumor foci and larger foci in CASTOR1 knockout mice than in mice with WT CASTOR1 (FIGS. 18A-18D). Tumors from CASTOR1 knockout mice have higher levels of mTORC1 activation and more mitotic cells than those from WT CASTOR1 mice as revealed by p4EBP1 and Ki67 staining, respectively (FIGS. 19A-19C). These results confirmed the tumor-suppressive function of CASTOR1 in a genetic cancer model and validated the AKT-CASTOR1-RNF167 axis as a viable cancer target.

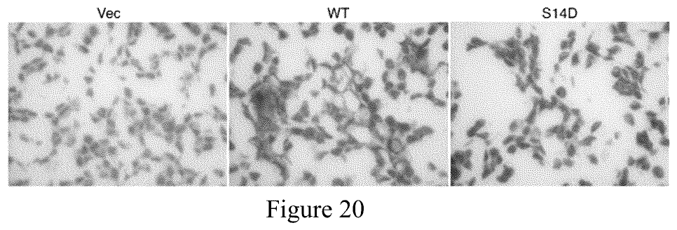

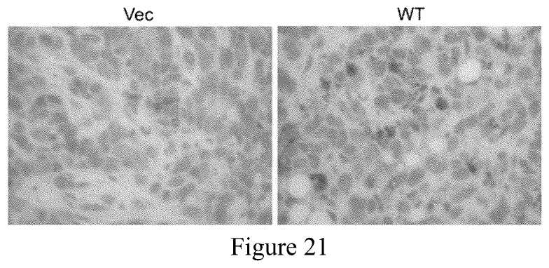

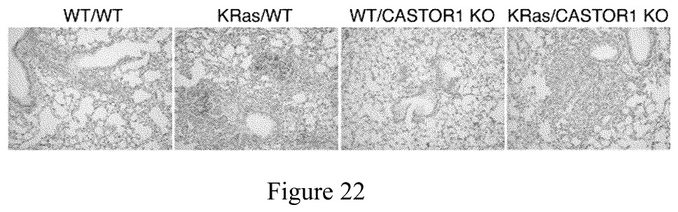

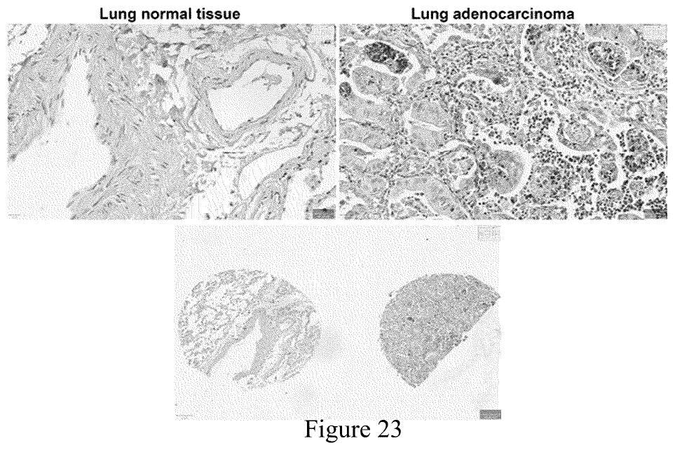

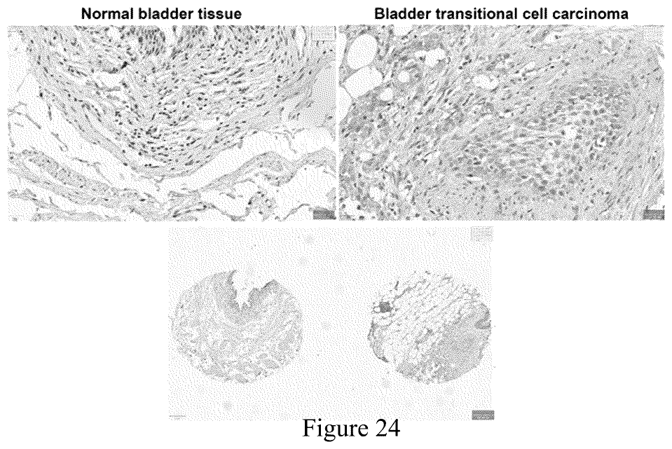

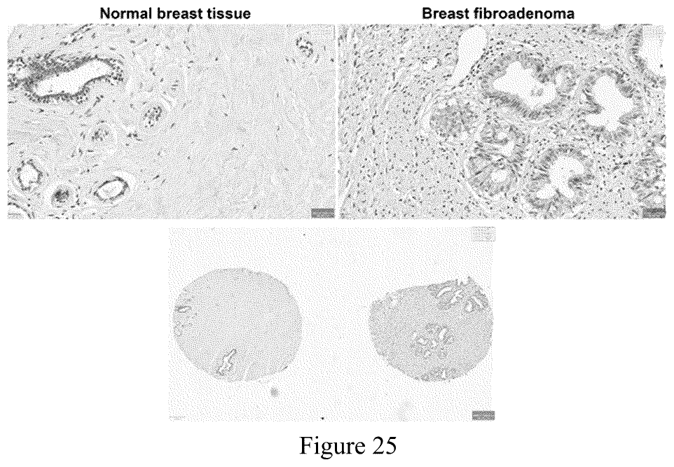

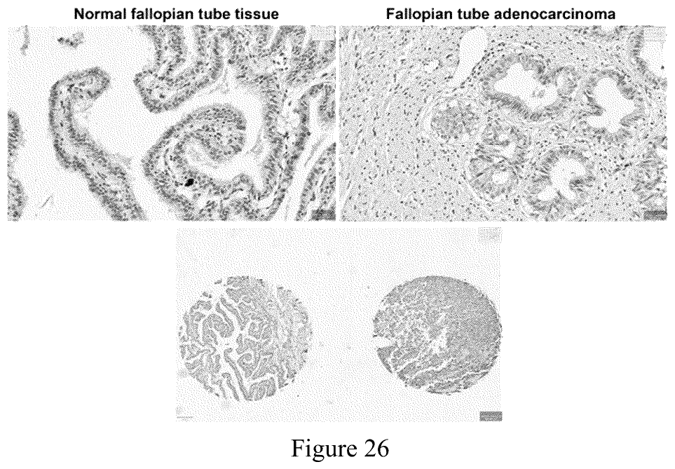

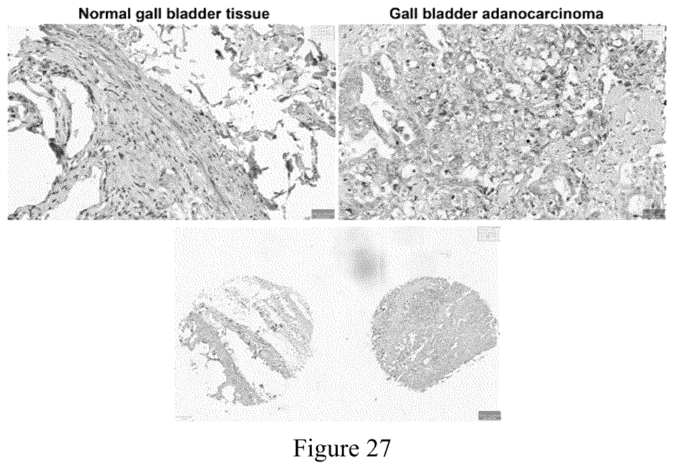

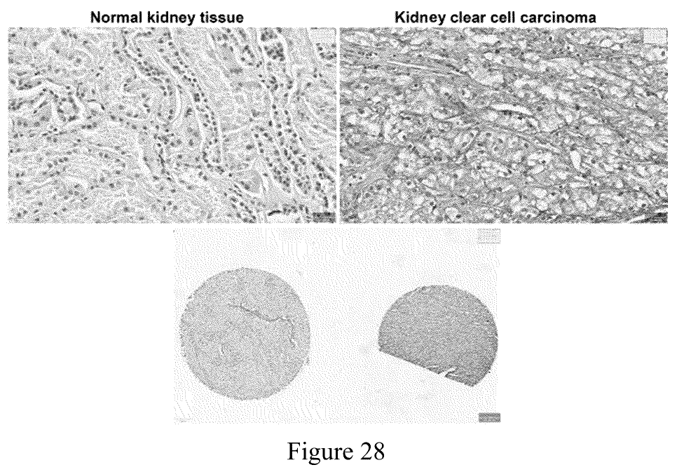

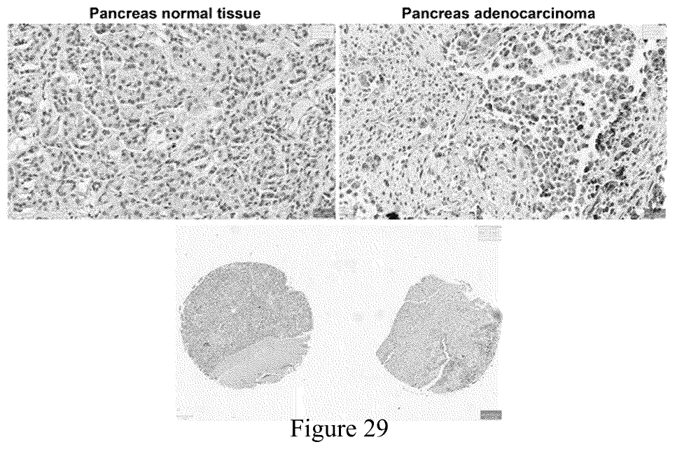

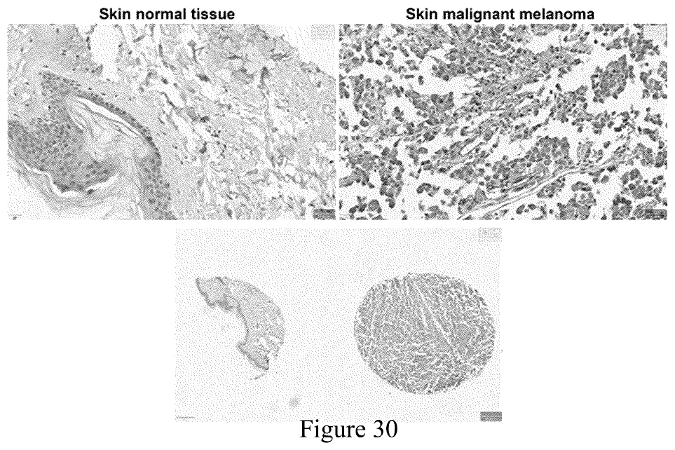

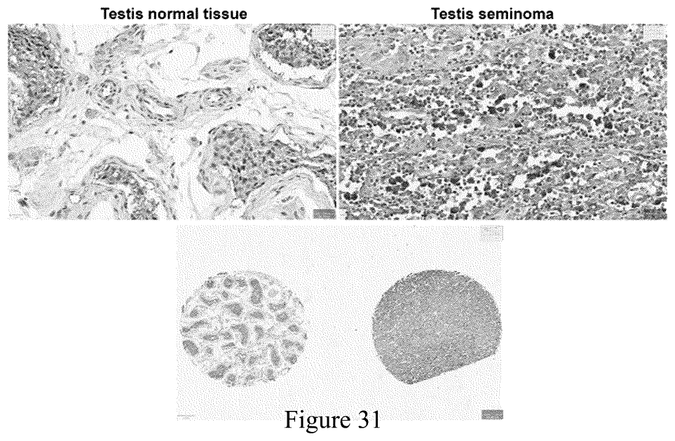

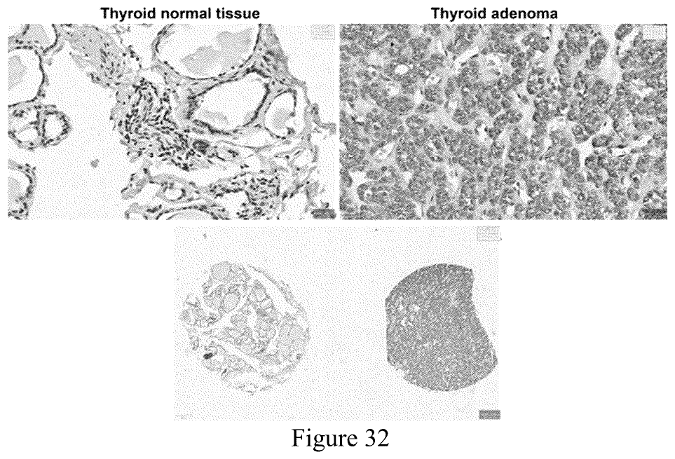

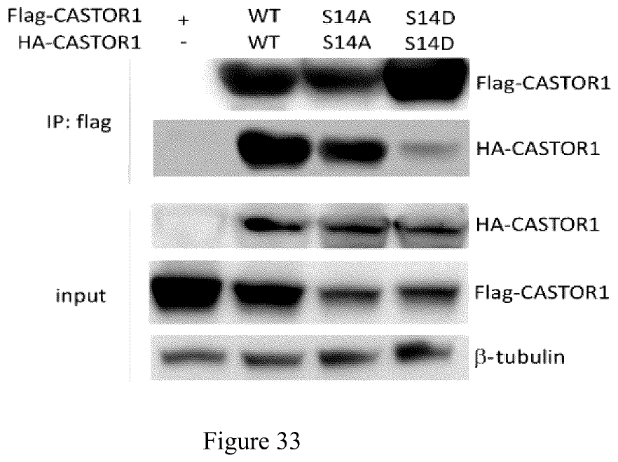

CASTOR1 phosphorylated at S14 (p-CASTOR1) as a biomarker: CASTOR1 S14 was identified as an AKT phosphorylated site. As described in Example 1, AKT-mediated CASTOR1 phosphorylation at S14 (p-CASTOR1) targets it for ubiquitination and degradation resulting in the activation of mTORC1. p-CASTOR1 is an indicator of mTORC1 activity and can be used as a biomarker for mTORC1 activity. Examination of several types of human cancer using a phospho-specific antibody for CASTOR1 phosphorylated at S14 show that p-CASTOR1 is detected at higher levels in tumors than the control normal tissues of a Kras-driven mouse lung cancer model (FIG. 22), lung adenocarcinoma (FIG. 23), bladder transitional cell carcinoma (FIG. 24), breast fibroadenoma (FIG. 25), fallopian tube adenocarcinoma (FIG. 26), gall bladder adenocarcinoma (FIG. 27), kidney clear cell carcinoma (FIG. 28), pancreas adenocarcinoma (FIG. 29), skin malignant melanoma (FIG. 30), testis seminoma (FIG. 31) and thyroid adenoma (FIG. 32). As a control p-CASTOR1 levels in MCF7 cells of WT and S14D CASTOR1 (FIG. 20) and xenograft tumors of MCF7 cells (FIG. 2I) were tested. These data confirm that p-CASTOR1 is upregulated in cancer tissue and can be used as a biomarker for prognosis.

### REFERENCES

Song M, Bode A M, Dong Z, Lee M H. AKT as a Therapeutic Target for Cancer. Cancer Res 79, 1019-1031 (2019).
Waks A G, Winer E P. Breast Cancer Treatment: A Review. JAMA 321, 288-300 (2019).
Manning B D, Toker A. AKT/PKB Signaling: Navigating the Network. Cell 169, 381-405 (2017).
Manning B D, Tee A R, Logsdon M N, Blenis J, Cantley L C. Identification of the tuberous sclerosis complex-2 tumor suppressor gene product tuberin as a target of the phosphoinositide 3-kinase/akt pathway. Mol Cell 10, 151-162 (2002).
Inoki K, Li Y, Zhu T, Wu J, Guan K L. TSC2 is phosphorylated and inhibited by Akt and suppresses mTOR signalling. Nat Cell Biol 4, 648-657 (2002).
Alessi D R, Caudwell F B, Andjelkovic M, Hemmings B A, Cohen P. Molecular basis for the substrate specificity of protein kinase B; comparison with MAPKAP kinase-1 and p70 S6 kinase. FEBS Lett 399, 333-338 (1996).
Chantranupong L, et al. The CASTOR Proteins Are Arginine Sensors for the mTORC1 Pathway. Cell 165, 153-164 (2016).
Saxton R A, Chantranupong L, Knockenhauer K E, Schwartz T U, Sabatini D M. Mechanism of arginine sensing by CASTOR1 upstream of mTORC1. Nature 536, 229-233 (2016).
Ward P S, Thompson C B. Metabolic reprogramming: a cancer hallmark even warburg did not anticipate. Cancer Cell 21, 297-308 (2012).
Delage B, et al. Arginine deprivation and argininosuccinate synthetase expression in the treatment of cancer. Int J Cancer 126, 2762-2772 (2010).
Bean G R, et al. A metabolic synthetic lethal strategy with arginine deprivation and chloroquine leads to cell death in ASS1-deficient sarcomas. Cell Death Dis 7, e2406 (2016).
Sabatini D M. mTOR and cancer: insights into a complex relationship. Nat Rev Cancer 6, 729-734 (2006).
Li T, Ju E, Gao S J. Kaposi sarcoma-associated herpesvirus miRNAs suppress CASTOR1-mediated mTORC1 inhibition to promote tumorigenesis. J Clin Invest 130, (2019).
Obenauer J C, Cantley L C, Yaffe M B. Scansite 2.0: Proteome-wide prediction of cell signaling interactions using short sequence motifs. Nucleic Acids Res 31, 3635-3641 (2003).
Hombeck P V, Zhang B, Murray B, Komhauser J M, Latham V, Skrzypek E. PhosphoSitePlus, 2014: mutations, PTMs and recalibrations. Nucleic Acids Res 43, D512-520 (2015).
Pandurangan A P, Ochoa-Montano B, Ascher D B, Blundell T L. SDM: a server for predicting effects of mutations on protein stability. Nucleic Acids Res 45, W229-W235 (2017).
Hunter T. The age of crosstalk: phosphorylation, ubiquitination, and beyond. Mol Cell 28, 730-738 (2007).
Pickart C M, Eddins M J. Ubiquitin: structures, functions, mechanisms. Biochim Biophys Acta 1695, 55-72 (2004).
Komander D, Rape M. The ubiquitin code. Annu Rev Biochem 81, 203-229 (2012).
Lussier M P, et al. Ubiquitin ligase RNF167 regulates AMPA receptor-mediated synaptic transmission. Proc Natl Acad Sci USA 109, 19426-19431 (2012).
Deshar R, Moon S, Yoo W, Cho E B, Yoon S K, Yoon J B. RNF167 targets Arl8B for degradation to regulate lysosome positioning and endocytic trafficking. FEBS J 283, 4583-4599 (2016).
Yamazaki Y, Schonherr C, Varshney G K, Dogru M, Hallberg B, Palmer R H. Goliath family E3 ligases regulate the recycling endosome pathway via VAMP3 ubiquitylation. EMBO J 32, 524-537 (2013).
Yoon S O, et al. Focal Adhesion- and IGF1R-Dependent Survival and Migratory Pathways Mediate Tumor Resistance to mTORC1/2 Inhibition. Molecular Cell 67, 512-+(2017).
Hara K, Yonezawa K, Weng Q P, Kozlowski M T, Belham C, Avruch J. Amino acid sufficiency and mTOR regulate p70 S6 kinase and eIF-4E BPl through a common effector mechanism. (vol 273, pg 14484, 1998). Journal of Biological Chemistry 273, 22160-22160 (1998).
Chen J, et al. KLHL22 activates amino-acid-dependent mTORC1 signalling to promote tumorigenesis and ageing. Nature 557, 585-589 (2018).
Jones T, et al. Direct and efficient cellular transformation of primary rat mesenchymal precursor cells by KSHV. J Clin Invest 122, 1076-1081 (2012).
Horman S R, et al. Akt-mediated phosphorylation of argonaute 2 downregulates cleavage and upregulates translational repression of microRNA targets. Mol Cell 50, 356-367 (2013).
Hanada M, Feng J, Hemmings B A. Structure, regulation and function of PKB/AKT—a major therapeutic target. Biochim Biophys Acta 1697, 3-16 (2004).
Radivojac P, et al. Identification, analysis, and prediction of protein ubiquitination sites. Proteins 78, 365-380 (2010).
Li T, Ju E, Gao S J. Kaposi sarcoma-associated herpesvirus miRNAs suppress CASTOR1-mediated mTORC1 inhibition to promote tumorigenesis. J Clin Invest 129, 3310-3323 (2019).
Zhou X F, et al. CASTOR1 suppresses the progression of lung adenocarcinoma and predicts poor prognosis. Journal of Cellular Biochemistry 119, 10186-10194 (2018).
Waks A G, Winer E P. Breast Cancer Treatment A Review. Jama-J Am Med Assoc 321, 288-300 (2019).
Costa RLB, Han H S, Gradishar W J. Targeting the PI3K/AKT/mTOR pathway in triple-negative breast cancer: a review. Breast Cancer Res Tr 169, 397-406 (2018).

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various publications and nucleic acid and amino acid sequence accession numbers are cited herein, the contents and full sequences of which are hereby incorporated by reference herein in their entireties.

What is claimed:

1. A method for treating a disease in a subject, comprising administering a therapeutically effective amount of a ring finger protein 167 (RNF167) inhibitor to the subject.

2. The method of claim 1, wherein the disease is diabetes or ageing.

3. The method of claim 1, wherein the disease is a cancer.

4. The method of claim 3, wherein the cancer is a breast cancer.

5. The method of claim 1, wherein the RNF167 inhibitor is selected from the group consisting of a compound, a small molecule, a chemical, a polypeptide, a peptide, a protein, and a combination thereof.

6. The method of claim 1, further comprising administering a therapeutically effective amount of an anti-cancer agent to the subject.

7. The method of claim 1, further comprising administering a therapeutically effective amount of an agent selected from the group consisting of a protein kinase B (AKT) inhibitor, a Tuberous Sclerosis Complex 2 (TSC2) inhibitor, an mTORC1 inhibitor and a combination thereof.

8. A method for treating a disease in a subject, comprising administering a therapeutically effective amount of an inhibitor that reduces phosphorylation of CASTOR1 at S14 and/or reduces degradation of CASTOR1 and/or an agonist of CASTOR1.

9. The method of claim 8, wherein the disease is diabetes or ageing.

10. The method of claim 8, wherein the disease is a cancer.

11. The method of claim 10, wherein the cancer is a breast cancer.

12. The method of claim 8, wherein the inhibitor that reduces phosphorylation of CASTOR1 at S14 and/or reduces degradation of CASTOR1 and/or the agonist of CASTOR1 is selected from the group consisting of a compound, a small molecule, a chemical, a polypeptide, a peptide, a protein, and a combination thereof.

13. The method of claim 8, further comprising administering a therapeutically effective amount of an anti-cancer agent to the subject.

14. The method of claim 8, further comprising administering a therapeutically effective amount of an agent selected from the group consisting of a protein kinase B (AKT) inhibitor, a Tuberous Sclerosis Complex 2 (TSC2) inhibitor, an mTORC1 inhibitor and a combination thereof.

15. A pharmaceutical composition for treating a disease in a subject, comprising a therapeutically effective amount of a ring finger protein 167 (RNF167) inhibitor.

16. The pharmaceutical composition of claim 15, wherein the RNF167 inhibitor is selected from the group consisting of a compound, a small molecule, a chemical, a polypeptide, a peptide, a protein, and a combination thereof.

17. A pharmaceutical composition for treating a disease in a subject, comprising a therapeutically effective amount of an inhibitor that reduces phosphorylation of CASTOR1 at S14 and/or reduces degradation of CASTOR1 and/or an agonist of CASTOR1.

18. The pharmaceutical composition of claim 17, wherein the inhibitor that reduces phosphorylation of CASTOR1 at S14 and/or reduces degradation of CASTOR1 and/or the agonist of CASTOR1 is selected from the group consisting of a compound, a small molecule, a chemical, a polypeptide, a peptide, a protein, and a combination thereof.


SEQUENCE LISTING (37 sequences)

SEQ ID NO: 1 - DNA, 65 bp, synthetic construct
  misc_feature 1..65: note = Description of Artificial Sequence: Synthetic primer
  source 1..65: mol_type = other DNA
tatgcggccgcgccaccatggactacaaagacgatgacgacaagatggaacttcacatccagagc

SEQ ID NO: 2 - DNA, 31 bp, synthetic construct
  misc_feature 1..31: note = Description of Artificial Sequence: Synthetic primer
  source 1..31: mol_type = other DNA
ataggatccctatggatctttggaagccagg

SEQ ID NO: 3 - DNA, 39 bp, synthetic construct
  misc_feature 1..39: note = Description of Artificial Sequence: Synthetic primer
  source 1..39: mol_type = other DNA
tatgcggccgcgccaccatggagctgcacatcctagaac

SEQ ID NO: 4 - DNA, 29 bp, synthetic construct
  misc_feature 1..29: note = Description of Artificial Sequence: Synthetic primer
  source 1..29: mol_type = other DNA
ataggatcctcaggaagccaggccttcct

SEQ ID NO: 5 - DNA, 69 bp, synthetic construct
  misc_feature 1..69: note = Description of Artificial Sequence: Synthetic primer
  source 1..69: mol_type = other DNA
tatgcggccgcgccaccatgtacccatacgatgttccagattacgctatggagctgcacatcctagaac

SEQ ID NO: 6 - DNA, 29 bp, synthetic construct
  misc_feature 1..29: note = Description of Artificial Sequence: Synthetic primer
  source 1..29: mol_type = other DNA
ataggatcctcaggaagccaggccttcct

SEQ ID NO: 7 - DNA, 66 bp, synthetic construct
  misc_feature 1..66: note = Description of Artificial Sequence: Synthetic primer
  source 1..66: mol_type = other DNA
tatgcggccgcgccaccatggactacaaagacgatgacgacaagatggagctgcacatcctagaac

SEQ ID NO: 8 - DNA, 29 bp, synthetic construct
  misc_feature 1..29: note = Description of Artificial Sequence: Synthetic primer
  source 1..29: mol_type = other DNA
ataggatcctcaggaagccaggccttcct

SEQ ID NO: 9 - DNA, 23 bp, synthetic construct
  misc_feature 1..23: note = Description of Artificial Sequence: Synthetic primer
  source 1..23: mol_type = other DNA
gcgggtgctggctgtcgcccgtc

SEQ ID NO: 10 - DNA, 18 bp, synthetic construct
  misc_feature 1..18: note = Description of Artificial Sequence: Synthetic primer
  source 1..18: mol_type = other DNA
acccggtgttctaggatg

SEQ ID NO: 11 - DNA, 23 bp, synthetic construct
  misc_feature 1..23: note = Description of Artificial Sequence: Synthetic primer
  source 1..23: mol_type = other DNA
gcgggtgctggatgtcgcccgtc

SEQ ID NO: 12 - DNA, 18 bp, synthetic construct
  misc_feature 1..18: note = Description of Artificial Sequence: Synthetic primer
  source 1..18: mol_type = other DNA
acccggtgttctaggatg

SEQ ID NO: 13 - DNA, 23 bp, synthetic construct
  misc_feature 1..23: note = Description of Artificial Sequence: Synthetic primer
  source 1..23: mol_type = other DNA
ggagggctttcgagagctgcccc

SEQ ID NO: 14 - DNA, 18 bp, synthetic construct
  misc_feature 1..18: note = Description of Artificial Sequence: Synthetic primer
  source 1..18: mol_type = other DNA
tcgtccaccataagcgtg

SEQ ID NO: 15 - DNA, 26 bp, synthetic construct
  misc_feature 1..26: note = Description of Artificial Sequence: Synthetic primer
  source 1..26: mol_type = other DNA
tggggtcacccggatcgcccgttcgg

SEQ ID NO: 16 - DNA, 18 bp, synthetic construct
  misc_feature 1..18: note = Description of Artificial Sequence: Synthetic primer
  source 1..18: mol_type = other DNA
gcagcctgcactgccgca

SEQ ID NO: 17 - DNA, 23 bp, synthetic construct
  misc_feature 1..23: note = Description of Artificial Sequence: Synthetic primer
  source 1..23: mol_type = other DNA
cagcaccccccgggaggcagcct

SEQ ID NO: 18 - DNA, 24 bp, synthetic construct
  misc_feature 1..24: note = Description of Artificial Sequence: Synthetic primer
  source 1..24: mol_type = other DNA
tgcgagtagaagaggacatctatg

SEQ ID NO: 19 - DNA, 21 bp, synthetic construct
  misc_feature 1..21: note = Description of Artificial Sequence: Synthetic primer
  source 1..21: mol_type = other DNA
ttgtactacacaaaagtactg

SEQ ID NO: 20 - DNA, 21 bp, synthetic construct
  misc_feature 1..21: note = Description of Artificial Sequence: Synthetic primer
  source 1..21: mol_type = other DNA
ggagctgcacatcctagaaca

SEQ ID NO: 21 - DNA, 21 bp, synthetic construct
  misc_feature 1..21: note = Description of Artificial Sequence: Synthetic primer
  source 1..21: mol_type = other DNA
gctttgatgaatgtggcatcg

SEQ ID NO: 22 - DNA, 20 bp, synthetic construct
  misc_feature 1..20: note = Description of Artificial Sequence: Synthetic primer
  source 1..20: mol_type = other DNA
gccaccaccctcatagatgt

SEQ ID NO: 23 - DNA, 20 bp, synthetic construct
  misc_feature 1..20: note = Description of Artificial Sequence: Synthetic primer
  source 1..20: mol_type = other DNA
aggaggtcactggggaactt

SEQ ID NO: 24 - DNA, 20 bp, synthetic construct
  misc_feature 1..20: note = Description of Artificial Sequence: Synthetic primer
  source 1..20: mol_type = other DNA
atcattgctcctcctgagcg

SEQ ID NO: 25 - DNA, 20 bp, synthetic construct
  misc_feature 1..20: note = Description of Artificial Sequence: Synthetic primer
  source 1..20: mol_type = other DNA
cggactcgtcatactcctgc

SEQ ID NO: 26 - protein, 6 aa, synthetic construct
  REGION 1..6: note = Description of Artificial Sequence: Synthetic peptide
RVRVLS

SEQ ID NO: 27 - protein, 22 aa, Xenopus tropicalis
MDLQLLEHRVRVTSIEKCGLWF

SEQ ID NO: 28 - protein, 22 aa, Gallus sp.
MELHILEHRVRVLSLARRGLWL

SEQ ID NO: 29 - protein, 22 aa, Rattus sp.
MELHILEHRVRVLSLARPGLWL

SEQ ID NO: 30 - protein, 22 aa, Canis lupus
MELHILEHRVRVLSLARPGLWL

SEQ ID NO: 31 - protein, 22 aa, Equus sp.
MELHILEHRVRVLSLARPGLWL

SEQ ID NO: 32 - protein, 22 aa, Macaca mulatta
MELHILEHRVRVLSVARPGLWL

SEQ ID NO: 33 - protein, 22 aa, Homo sapiens
MELHILEHRVRVLSVARPGLWL

SEQ ID NO: 34 - protein, 22 aa, Gorilla sp.
MELHILEHRVRVLSVARPGLWL

SEQ ID NO: 35 - protein, 22 aa, Pan troglodytes
MELHILEHRVRVLSVARPGLWL

SEQ ID NO: 36 - protein, 22 aa, Bos sp.
MELHILEHRVRVLSLARPGLWL

SEQ ID NO: 37 - protein, 22 aa, Sus sp.
MELHILEHRVRVLSLARPGLWL